(19) United States
(12) Patent Application Publication
(10) Pub. No.: US 2018/0221508 A1
(43) Pub. Date: Aug. 9, 2018

(54) COMPOSITIONS AND METHODS FOR IMMUNOMODULATION

(71) Applicant: TARVEDA THERAPEUTICS, INC., Watertown, MA (US)

(72) Inventors: Sudhakar KADIYALA, Newton, MA (US); Donna T. WARD, Groton, MA (US)

(21) Appl. No.: 15/749,194

(22) PCT Filed: Jul. 29, 2016
(86) PCT No.: PCT/US2016/044705

§ 371 Date: Jan. 31, 2018

Related U.S. Application Data
(60) Provisional application No. 62/199,422, filed on Jul. 31, 2015.
(60) Provisional application No. 62/332,838, filed on May 6, 2016.

(51) Int. Cl. A61K 47/68; A61P 35/00; A61P 37/04; C07K 16/28; C07K 16/46; C07K 16/24; C07K 16/22; C07K 16/30; A61K 41/00; A61K 47/69

(52) U.S. Cl. CPC A61K 47/6881; A61K 2039/505; A61P 37/04; A61K 47/6889; A61K 47/6851; A61K 47/6849; C07K 16/2818; C07K 16/468; A61K 47/6879; C07K 16/2866; C07K 16/244; C07K 16/22; A61K 47/6845; A61K 47/6843; C07K 16/30; A61K 41/0042; A61K 47/6935; A61K 47/6937; C07K 2319/30; C07K 2317/76; C07K 2317/75; C07K 2317/622; C07K 2317/31; A61P 35/00

(57) ABSTRACT

The present invention relates to modulation of the tumor microenvironment to increase cancer specific immune responses. Conjugates, nanoparticles and formulations of the present invention relieve the inhibitory effect induced by tumor cells, and enhance antitumor immunity. The compostions provided herein can be used as immunotherapies, or as adjuvants used in conjunction with other immunotherapies such as peptide vaccines, cell vaccines and/or adoptive T cell transfer.

### REFERENCE TO RELATED APPLICATIONS

The present application claims priority to U.S. Provisional Patent Application No. 62/199,422, filed Jul. 31, 2015, entitled COMPOSITIONS AND METHODS FOR IMMUNOMODULATION, and U.S. Provisional Patent Application No. 62/332,838, filed May 6, 2016, entitled COMPOSITIONS AND METHODS FOR IMMUNOMODULATION, the contents of each of which are herein incorporated by reference in their entirety.

### FIELD OF THE INVENTION

The present invention is generally in the field of immuno-oncology therapy. In particular, the present invention relates to inhibition of immunosuppression for enhancing immunotherapy efficacy. Conjugates comprising one or more active agents that are involved in modulating immunosuppression, nanoparticles, and formulations packaging such conjugates are provided.

### BACKGROUND OF THE INVENTION

Immunotherapy holds much promise for treatment of cancer. A wide variety of approaches have been implemented in order to stimulate a range of immune responses including innate and adaptive immune activities, to eliminate cancer cells. Strategies used to boost a specific anti-cancer immune response include tumor specific antigen/peptide vaccines, dendritic cell vaccines, adoptive T cell transfer and other positive immunomodulatory adjuvants. However, some clinical trials and researches conducted on experimental models indicated that some immunotherapeutic approaches are of limited values against some cancers. Recent studies have suggested that the lack of an effective immune reactivity to tumors may be explained by the immune tolerance and suppression induced by tumor cells.

It has been shown that the immune system itself is tightly regulated to avoid overactivation of its defensive function, such as autoimmunity, through a variety of regulatory immune cells and cell-expressed or secreted immunomodulatory molecules.

In many cancers, tumor cells can regulate cancer microenvironment locally, leading to an ineffective and suppressed tumor microenvironment, therefore to allow them to escape the immune surveillance. Anti-cancer immunity within the tumor microenvironment can be suppressed by a variety of tumor infiltrating leukocytes including regulatory T cells (Whiteside, induced regulatory T cells in inhibitory microenvironments created by cancer, Expert Opin Biol Ther., 2014, 14(10): 1411-1425), myeloid-derived suppressor cells (MDSCs) (Lee et al., Elevated endoplasmic reticulum stress reinforced immunosuppression in the tumor microenvironment via myeloid-derived suppressor cells, Oncotarget, 2014, 5(23): 12331-12345), and alternatively activated macrophages (types) (M2) (Chanmee et al., Tumor associated macrophages as major players in the tumor microenvironment. Cancers (Basel), 2014, 6(3): 1670-1679). These tumor infiltrating cells can secret many inhibitory cytokines such as IL-10 and TGF-β and amino acid-depleting enzymes such as arginase and IDO, or express inhibitory receptors such as programmed cell death protein 1 (PD-1, also known as CD279) and cytotoxic T⁻lymphocyte-associated antigen 4 (CTLA-4, also known as CD152). These negative molecules can pose significantly impact on anti-tumor immunity.

Additionally, tumor cells themselves can actively inhibit tumor immunity through a number of mechanisms. Tumor cells can block activated T cells by secreting soluble ligands for receptors expressed on the surface of activated cancer specific T cells, such as soluble MICA and MICB ligands for receptor CD134/NKG2D (Groh et al., Tumor-derived soluble MIC ligands impair expression of NKG2D and T cell activation. Nature, 2002, 419: 734-738). Additionally, tumor cells can secret cytokines to impact T cell activity. For example, tumor cell secreted TGF-β, VEGF, IL-10 and galectins can impede T cell activity and survival (Rubinstein et al., targeted inhibition of galentin-1 gene expression in tumor cells results in heightened T cell-mediated rejection; a potential mechanism of tumor immune privilege. Cancer cell, 2004, 5: 241-251).

Many of these inhibitory mechanisms within the tumor microenvironment result in a strong immune suppression. Approaches that specifically reduce or inhibit immune suppression within the tumor microenvironment, alone, or in combination with immunotherapy that aims to provoke an anti-cancer immune response will be valuable strategies to increase the efficacy of cancer immunotherapy.

The present invention focuses on immune-based approaches to change the tumor microenvironment to enable anti-cancer immune responses. Provided in the present invention are conjugates comprising one or more active agents that are involved in modulating the tumor microenvironment, in particular, inhibiting the immune suppression mechanisms in the tumor microenvironment. Nanoparticles and formulations comprising the present conjugates are also provided.

### SUMMARY OF THE INVENTION

The present invention provides novel conjugates comprising at least one active agent that modulates the tumor microenvironment, and a targeting moiety that targets to a specific cell, or a specific site, of the interest, wherein the active agent and the targeting moiety is connected through a linker, or in some instances, directly linked to each other. The targeting moiety increases the delivery and biodistribution of the active agent in a targeted area. The linker can be used to control the release of the active agent to the targeted site.

In some embodiments, the active agent of the conjugate can inhibit the immunosuppression mechanisms induced by tumor cells and negative immune regulatory cells in the tumor microenvironment. The active agent may be an antagonistic agent specific to a coinhibitory checkpoint molecule that can antagonize or reduce the inhibitory signal to effector immune cells (e.g. cytotoxic T cells and natural killer cells). In other aspects, the active agent may be an inhibitor that can inhibits and reduces the activity of immune suppressive enzymes (e.g. ARG and IDO) and cytokines (e.g. IL-10), chemokines and other soluble factors (e.g., TGF-β and VEGF) in the tumor microenvironment.

### DETAILED DESCRIPTION OF THE INVENTION

The details of one or more embodiments of the invention are set forth in the accompanying description below. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

### The Tumor Microenvironment

Tumor cells can induce an immunosuppressive microenvironment to help them escape the immune surveillance. The immune suppression in the tumor microenvironment is either induced by intrinsic immune suppression mechanisms, or directly by tumors.

In adaptive immune responses for eliminating tumor cells, cytotoxic T cell activation needs both a primary signal from a specific antigen (i.e. first signal) and one or more co-stimulatory signals (i.e. secondary signal). Antigen presenting cells (e.g., dendritic cells) process tumor associated antigens (TAAs) and present antigenic peptides derived from TAAs (i.e. epitopes) on the cell surface as peptide/MHC molecule (class I/II) (p/MHC) complexes and T cells engage APCs loaded with TAAs via their T cell receptors (TCRs) which recognize the p/MHC complexes. This ligation is the primary signal to activate cancer specific cytotoxic T cells. Additionally, a secondary co-stimulating signal is provided by co-stimulatory receptors on the T cells and their ligands (or coreceptors) on the APCs. The interaction between co-stimulatory receptors and their ligands can regulate T cell activation and enhance its activity. CD28, 4-1BB (CD137), and OX40 are well studied co-stimulatory receptors on T cells, which bind to B7-1/2 (CD80/CD86), 4-1BB (CD137L) and OX-40L, respectively on APCs. In normal circumstance, to prevent excessive T-cell proliferation and balance the immunity, a co-inhibitory signal, e.g., CTLA-4, can be induced and expressed by activated T cells and competes with CD28 in binding to B7 ligands on APCs. This can mitigate a T cell response in a normal circumstance. However, in some cancers, tumor cells and regulatory T cells infiltrating the tumor microenvironment can constitutively express CTLA-4. This co-inhibitory signal suppresses the co-stimulatory signal, therefore, depleting an anti-cancer immune response.

In addition to CTLA-4 signal, activated T cells can also be induced to express another inhibitory receptor, PD-1 (programed death 1). In normal situation, as an immune response progresses, CD4+ and CD8+ T lymphocytes upregulate the expression of these inhibitory checkpoint receptors (e.g., PD-1). Inflammatory conditions prompt IFN release, which will upregulate the expression of PD-1 ligands: PD-L1 (also known as B7-H1) and PD-L2 (also known as B7-DC) in peripheral tissues, to maintain immune tolerance to prevent autoimmunity. Many human cancer types have been demonstrated to express PD-L1 in the tumor microenvironment (e.g., Zou and Chen, inhibitory B7-family in the tumor microenvironment. 2008, Nat Rev Immunol, 8: 467-477). The PD-1/PD-L1 interaction is highly active with the tumor microenvironment, inhibiting T cell activation.

Other identified co-inhibitory signals in the tumor microenvironment include TIM-3, LAG-3, BTLA, CD160, CD200R, TIGIT, KLRG-1, KIR, CD244/2B4, VISTA and Ara2R.

In addition, the tumor microenvironment contains suppressive elements including regulatory T cells (Treg), myeloid-derived suppressor cells (MDSC) and tumor-associated macrophage (TAM); soluble factors such as interleukin 6 (IL-6), IL-10, vascular endothelial growth factor (VEGF), and transforming growth factor beta (TGF-β). An important mechanism by which IL-10, TGF-β, and VEGF counteract the development of an anti-cancer immune response is through inhibition of dendritic cell (DC) differentiation, maturation, trafficking, and antigen presentation (Gabrilovich D: Mechanisms and functional significance of tumour-induced dendritic-cell defects, Nat Rev Immunol, 2004, 4: 941-952).

Regulatory T cells (Treg): CD4+CD25+ Treg cells represent a unique population of lymphocytes that are thymus-derived. CD4+CD25+ Treg cells, which were marked by forkhead box transcription factor (Foxp3), play a critical role in maintaining self-tolerance, suppress autoimmunity and regulate immune responses in organ transplantation and tumor immunity. Tumor development often attracts CD4+CD25+ FoxP3+ Treg cells to the tumor area. Tumor infiltrating regulatory T cells secret inhibitory cytokines such as IL-10 and TGFβ to inhibit autoimmune and chronic inflammatory responses and to maintain immune tolerance in tumors (Unitt et al., Compromised lymphocytes infiltrate hepatocellular carcinoma: the role of T-regulatory cells. Hepatology. 2005; 41(4):722-730).

Myeloid derived suppressor cells (MDSCs): MDSCs are a group of heterogeneous cells, which could be seen as hallmark of malignancy-associated inflammation and a major mediator for the induction of T cell suppression in cancers. MDSCs are found in many malignant areas and divided phenotypically into granulocytic (G-MDSC) and monocytic (Mo-MDSC) subgroups. MDSCs can induce T regulatory cells, and produce T cell tolerance. Additionally MDSCs secrete TFG-β and IL-10 and produce nitric oxide (NO) in the presence of IFN-γ or activated T cells.

Tumor associated macrophage (TAM): TAMs derived from peripheral blood monocytes are multi-functional cells which exhibit different functions to different signals from the tumor microenvironment. Among cell types associated with tumor microenvironment, TAMs are the most influential for tumor progression. In response to microenvironmental stimuli, such as tumor extracellular matrix, anoxic environment and cytokines secreted by tumor cells, macrophages undergo M1 (classical) or M2 (alternative) activation. In most malignant tumors, TAMs have the phenotype of M2 macrophages.

Another immune suppressive mechanisms relate to tryptophan catabolism by the enzyme indoleamine-2,3-dioxygenase (IDO). Local immune suppression is an active process induced by the malignant cells within the tumor microenvironment and within the sentinel lymph nodes (SLN). (Gajewski et al., Immune suppression in the tumor microenvironment. J Immunother, 2006; 29(3):233-240; and Zou W., Immunosuppressive networks in the tumor environment and their therapeutic relevance, Nat Rev Cancer, 2005; 5(4):263-274). Studies show that T-cell receptor zeta subunit (TCR) is downregulated and Indoleamine 2,3-dioxygenase (IDO) is upregulated within the tumor draining lymph nodes as part of the elements involved in the regional immune suppression.

In addition to the suppressive effects medicated by infiltrating regulatory immune cells, tumor cells themselves can secret many molecules to actively inhibit cytotoxic T cell activation and function.

In some tumors, T cell intrinsic anergy and exhaustion is common, resulting from TCR ligation in the absence of engagement of co-stimulatory receptors on T cells such as CD28.

Inhibiting one or more immunosuppressive mechanisms, either as active treatment approaches, or adjuvants of cancer vaccination and adoptive T cell transfer, can enhance a cancer specific immune response for eliminating tumor cells.

Conjugates, nanoparticles and formulations of the present invention provide useful carriers for conjugating active agents that can release such immunosuppressive signals in the tumor microenvironment, through a linker, to a targeting moiety that targets to specific tissues and/or cells. Such conjugates increase targeted delivery of active agents and provide a controlled release of active agent for optimized outcomes.

### Compositions of the Invention

Compositions of the present inventions include conjugates comprising a targeting moiety, a linker, and one or more active agents, e.g., one or more immunoregulatory agents that may conjugated to the targeting moiety through a linker. Nanoparticles that package one or more conjugates of the present invention are also provided. The conjugates can be encapsulated into nanoparticles or disposed on the surface of the nanoparticles. In particular, conjugates of the present invention and nanoparticles comprising such conjugates may be used as immuno-oncology therapeutic agents such as checkpoint inhibitors and vaccine adjuvants. The conjugates, nanoparticles comprising the conjugates, and/or formulations thereof can provide improved temporospatial delivery of the active agent and/or improved biodistribution compared to delivery of the active agent alone.

### I. Conjugates of the Invention

In accordance with the present invention, conjugates comprise at least three moieties: a targeting moiety (or ligand), a linker, and an active agent called a payload that is connected to the targeting moiety via the linker. In some embodiments, the conjugate may be a conjugate between a single active agent and a single targeting moiety with the formula: X—Y—Z, wherein X is the targeting moiety; Y is a linker; and Z is the active agent. In certain embodiments, one targeting ligand can be conjugated to two or more payloads wherein the conjugate has the formula: X—(Y—Z)ₙ. In certain embodiments, one active payload can be linked to two or more targeting ligands wherein the conjugate has the formula: (X—Y)ₙ—Z. In other embodiments, one or more targeting ligands may be connected to one or more active payloads wherein the conjugate formula may be (X—Y—Z)n. In various combinations, the formula of the conjugates maybe, for example, X—Y—Z—Y—X, (X—Y—Z)ₙ—Y—Z, or X—Y—(X—Y—Z)ₙ, wherein X is a targeting moiety; Y is a linker; Z is an active agent. The number of each moiety in the conjugate may vary dependent on types of agents, sizes of the conjugate, delivery targets, particles used to packaging the conjugate, other active agents (e.g., immunologic adjuvants) and routes of administration. Each occurrence of X, Y, and Z can be the same or different, e.g. the conjugate can contain more than one type of targeting moiety, more than one type of linker, and/or more than one type of active agent. n is an integer equal to or greater than 1. In some embodiments, n is an integer between 1 and 50, or between 2 and 20, or between 5 and 40. In some embodiments, n may be an integer of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 41, 43, 44, 45, 46, 47, 48, 49 or 50.

In some embodiments, the conjugate may comprise pendent or terminal functional groups that allow further modification or conjugation. The pendent or terminal functional groups may be protected with any suitable protecting groups.

Conjugates of the present invention can modulate the immune suppression mechanisms in the tumor microenvironment.

In some embodiments, the conjugate of the present invention comprises a targeting moiety X, wherein X binds to a tumor cell; a linker Y; and an active agent Z that binds to a checkpoint receptor on T cells or natural killer cells. The conjugate may have a structure of X—Y—Z. The checkpoint receptor is selected from the group consisting of CTLA-4, PD-1, CD28, inducible T cell co-stimulator (ICOS), B and T lymphocyte attenuator (BTLA), killer cell immunoglobulinlike receptor (KIR), lymphocyte activation gene 3 (LAG3), CD137, OX40, CD27, CD40L, T cell membrane protein 3 (TIM3), and adenosine A2a receptor (A2aR). The active agent Z may be an antibody, antagonist, or a functional fragment thereof that binds to the checkpoint receptor and blocks the checkpoint pathway. The targeting moiety X may bind to a cell surface protein on tumor cells.

In some embodiments, the conjugate of the present invention comprises a targeting moiety X, wherein X binds to a checkpoint ligand on a tumor cell; a linker Y; and an active agent Z. The conjugate may have a structure of X—Y—Z. The checkpoint ligand is located on tumor cells and is selected from the group consisting of PD1 ligand-1 (PDL-1, also known as B7-H1), PD1 ligand 2 (PDL-2, also known as B7-DC), CD80, CD86, B7-related protein 1 (B7RP1), B7-H3 (also known as CD276), B7-H4 (also known as B7—S1, B7x and VCTN1), herpesvirus entry mediator (HVEM), CD137L, OX40L, CD70, CD40, and galectin 9 (GAL9S). The targeting moiety may be an antibody, antagonist, or a functional fragment thereof that binds to the checkpoint ligand and blocks the checkpoint pathway. The active agent may be an anti-cancer agent, an antigen that activates T cells, or a T cell binding moiety.

### A. Payloads

As used herein, the terms “payload” and “active agent” are used interchangeably. A payload may be any active agents such as therapeutic agents, prophylactic agents, or diagnostic/prognostic agents. A payload may have a capability of manipulating a physiological function (e.g., immune response) in a subject. One payload may be included in the present conjugate. One or more, either the same or different payloads may be included in the present conjugate.

In accordance with the present invention, a payload may be an active agent that targets immunological barriers in the tumor microenvironment to block one or more immune suppression mechanisms, therefore to provoke/enhance an anti-cancer immune response in a subject. Immunotherapy is an advantageous strategy to treat cancer. Any compound that can provoke a subject immune response to destroy tumor cells may be included in the conjugates.

### A. Checkpoint Inhibitors

During adaptive immune response, activation of cytotoxic T cells is mediated by a primary signal between antigenic peptide/MHC molecule complexes on antigen presenting cells and the T cell receptor (TCR) on T cells. A secondary co-stimulatory signal is also important to active T cells. Antigen presentation in the absence of the secondary signal is not sufficient to activate T cells, for example CD4+ T helper cells. The well-known co-stimulatory signal involves co-stimulatory receptor CD28 on T cells and its ligands B7-1/CD80 and B7-2/CD86 on antigen presenting cells (APCs). The B7-1/2 and CD28 interaction can augment antigen specific T cell proliferation and cytokine production. To tightly regulate an immune response, T cells also express CTLA-4 (anti-cytotoxic T-lymphocyte antigen 4), a co-inhibitory competitor of CD80 and CD86 mediated co-stimulation through the receptor CD28 on T cells, which can effectively inhibit T cell activation and function. CTLA-4 expression is often induced when CD28 interacts with B7-1/2 on the surface of an APC. CTLA-4 has higher binding affinity to the co-stimulatory ligand B7-1/2 (CD80/CD86) than the co-stimulatory receptor CD28, and therefore tips the balance from the T cell activating interaction between CD28 and B7-1/2 to inhibitory signaling between CTLA-4 and B7-1/2, leading to suppression of T cell activation. CTLA-4 upregulation is predominantly during the initial activation of T cells in the lymph node.

Antibodies that specifically bind to CTLA-4 have been used to inhibit this inhibitory checkpoint. The anti CTLA-4 IgG1 humanized antibody: ipilimumab binds to CTLA-4 and prevents the inhibition of CD28/B7 stimulatory signaling. They can lower the threshold for activation of T cells in lymphoid organs, also can deplete T regulatory cells within the tumor microenvironment (Simpson et al., Fc-dependent depletion of tumor-infiltrating regulatory T cells co-defines the efficacy of anti-CTLA-4 therapy against melanoma. J Exp. Med., 2013, 210: 1695-1710). Ipilimumab was recently approved by the U.S. Food and Drug Administration for the treatment of patients with metastatic melanoma.

In some embodiments, the payload of the conjugate of the present invention may be an antagonist agent against CTLA-4 such as an antibody, a functional fragment of the antibody, a polypeptide, or a functional fragment of the polypeptide, or a peptide, which can bind to CTLA-4 with high affinity and prevent the interaction of B7-1/2 (CD80/86) with CTLA-4. In one example. The CTLA-4 antagonist is an antagonistic antibody, or a functional fragment thereof. Suitable anti-CTLA-4 antagonistic antibody include, without limitation, anti-CTLA-4 antibodies, human anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (ipilimumab), tremelimumab (fully humanized), anti-CD28 antibodies, anti-CTLA-4 adnectins, anti-CTLA-4 domain antibodies, single chain anti-CTLA-4 antibody fragments, heavy chain anti-CTLA-4 fragments, light chain anti-CTLA-4 fragments, and the antibodies disclosed in U.S. Pat. Nos.: 8,748,815; 8,529,902; 8,318,916; 8,017,114; 7,744,875; 7,605,238; 7,465,446; 7,109,003; 7,132,281; 6,984,720; 6,682,736; 6,207,156; 5,977,318; and European Patent No. EP1212422B1; and U.S. Publication Nos. US 2002/0039581 and US 2002/086014; and Hurwitz et al., Proc. Natl. Acad. Sci. USA, 1998, 95(17):10067-10071; the contents of each of which are incorporated by reference herein in their entirety.

Additional anti-CTLA-4 antagonist agents include, but are not limited to, any inhibitors that are capable of disrupting the ability of CTLA-4 to bind to the ligands CD80/86.

The inhibitory receptor PD-1 (programmed death-1) is expressed on activated T cells and can induce inhibition and apoptosis of T cells following ligation by programmed death ligands 1 and 2 (PD-L1, also known as B7-H1, CD274), and PD-L2 (also known as B7-DC, CD273), which are normally expressed on epithelial cells and endothelial cells and immune cells (e.g., DCs, macrophages and B cells). PD-1 modulates T cell function mainly during the effector phase in peripheral tissues including tumor tissues. PD-1 is expressed on B cells and myeloid cells, in addition to activated T cells. Many human tumor cells can express PD-L1 and hijack this regulatory function to evade immune recognition and destruction by cytotoxic T lymphocytes. Tumor-associated PD-L1 has been shown to induce apoptosis of effector T cells and is thought to contribute to immune evasion by cancers.

The PD-1/PD-L1 immune checkpoint appears to be involved in multiple tumor types, for example, melanoma. PD-L1 not only provides immune escape for tumor cells but also turns on the apoptosis switch on activated T cells. Therapies that block this interaction have demonstrated promising clinical activity in several tumor types.

Agents used for blocking the PD-1 pathway include antagonistic peptides/antibodies and soluble PD-L1 ligands (See Table 1).

TABLE 1
Agents that block the inhibitory PD-1 and PD-L1 pathway

| Agent | Description | Target |
|---|---|---|
| Nivolumab | Human IgG | PD-1 |
| (BMS-936558, ONO-4538, | | |
| MDX-1106 | | |
| Pembrolizumab | Humanized IgG4 | PD-1 |
| (MK-3475, lambrolizumab) | | |
| Pidilizumab (CT-011) | Humanized anti-PD-1 | PD-1 |
| | IgG1kappa | |
| AMP-224 | B7-DC/IgG1 fusion | PD-1 |
| | protein | |
| MSB0010718 (EMD-Serono) | Human IgG1 | PD-L1 |
| MEDI4736 | Engineered human IgG | PD-L1 |
| | 1kappa | |
| MPDL3280A | Engineered IgG1 | PD-L1 |
| AUNP-12 | branched 29-amino | PD-1 |
| | acid peptide | |

In accordance with the present invention, the payload of the conjugate, in some embodiments, may be may be an antagonist agent against PD-1 and PD-L1/2 inhibitory pathway. In one embodiment, the antagonist agent may be an antagonistic antibody that specifically binds to PD-1 or PD-L1/L2 with high affinity, or a functional fragment thereof. The PD-1 antibodies may be antibodies taught in U.S. Pat. Nos: 8,779,105; 8,168,757; 8,008,449; 7,488,802; 6,808,710; and PCT publication No.: WO 2012/145493; the contents of which are incorporated by references herein in their entirety. Antibodies that can specifically bind to PD-L1 with high affinity may be those disclosed in U.S. Pat. Nos.: 8,552,154; 8,217,149; 7,943,743; 7,635,757; U.S. Publication No. 2009/0317368, and PCT Publication Nos. WO 2011/066389 and WO 2012/145493; the contents of which are incorporated herein by references in their entirety. In some examples, the payload of the conjugate may be an antibody selected from 17D8, 2D3, 4H1, 5C4 (also known as nivolumab or BMS-936558), 4A11, 7D3 and 5F4 disclosed in U.S. Pat. No.: 8,008,449; AMP-224, Pidilizumab (CT-011), and Pembrolizumab. In other examples, the anti-PD-1 antibody may be a variant of a human monoclonal anti-PD-1 antibody, for example a “mixed and matched” antibody variant in which a V_{H} sequence from a particular V_{H}/V_{L} pairing is replaced with a structurally similar V_{H} sequence, or a V_{L} sequence from a particular V_{H}/VL pairing is replaced with a structurally similar V_{L} sequence, as disclosed in US publication NO.: 2015/125463; the contents of which are incorporated by reference herein in its entirety.

In some embodiments, the payload of the conjugate may be an antagonistic antibody that binds to PD-L1 with high affinity and disrupts the interaction between PD-1/PD-L1/2. Such antibodies may include, without limitation, 3G10, 12A4 (also referred to as BMS-936559), 10A5, 5F8, 10H10, 1B12, 7H1, 11E6, 12B7, and 13G4 disclosed in U.S. Pat. No.: 7,943, 743 (the contents of which are incorporated by reference in its entirety), MPDL3280A, MEDI4736, and MSB0010718. In another example, the anti-PD-L1 antibody may be a variant of a human monoclonal anti-PD-L1 antibody, for example a “mixed and matched” antibody variant in which a V_{H} sequence from a particular V_{H}/V_{L} pairing is replaced with a structurally similar V_{H} sequence, or a V_{L} sequence from a particular V_{H}/V_{L} pairing is replaced with a structurally similar V_{L} sequence, as disclosed in US publication NO.: 2015/125463; the contents of which are incorporated by reference in its entirety.

In some embodiments, the payload of the conjugate may be an antagonistic antibody that binds to PD-L2 with high affinity and disrupts the interaction between PD-1/PD-L1/2. Exemplary anti-PD-L2 antibodies may include, without limitation, antibodies taught by Rozali et al (Rozali et al., Programmed Death Ligand 2 in Cancer-Induced Immune Suppression, Clinical and Developmental Immunology, 2012, Volume 2012 (2012), Article ID 656340), and human anti-PD-L2 antibodies disclosed in U.S. Pat. No.: 8,552,154 (the contents of which are incorporated herein by reference in their entirety).

In some embodiments, the payload of the conjugate may compounds that inhibit immunosuppressive signal induced due to PD-1, PD-L1 and/or PD-L2 such as cyclic peptidomimetic compounds disclosed in U.S. Pat. No. 9,233,940 to Sasikumar et al. (Aurigene Discovery Tech.), WO2015033303 to Sasikumar et al.; immunomodulating peptidomimetic compounds disclosed in WO2015036927 to Sasikumar et al.; 1,2,4-oxadiazole derivatives disclosed in US2015007302 to Govindan et al.; 1,3,4-oxadiazole and 1,3,4-thiadiazole compounds disclosed in WO2015033301 to Sasikumar et al.; or therapeutic immunomodulating compounds and derivatives or pharmaceutical salts of a peptide derivative of formula (I) or a stereoisomer of a peptide derivative of formula (I) disclosed in WO2015044900 to Sasikumar et al., the contents of each of which are incorporated herein by reference in their entirety.

In other embodiments, the payload of the conjugate may be an antibody having binding affinity to both PD-L1 and PD-L2 ligands, for example the single agent of anti-PD-L1 and PD-L2 antibodies disclosed in PCT publication NO.: WO2014/022758; the contents of which are incorporated by reference in its entirety.

In some embodiments, the conjugate of the present invention may comprise two or more antibodies selected from anti-PD-1 antibodies, PD-L1 antibodies and PD-L2 antibodies. In one example, an anti-PD-L1 antibody and an anti-PD-L2 antibody may be included in a single conjugate through the linkers to the targeting moiety.

In some embodiments, the payload of the conjugate may be a modulatory agent that can simultaneously block the PD-1 and PD-L1/2 mediated negative signal transduction. This modulatory agent may be a non-antibody agent. In some aspects, the non-antibody agents may be PD-L1 proteins, soluble PD-L1 fragments, variants and fusion proteins thereof. The non-antibody agents may be PD-L2 proteins, soluble PD-L2 fragments, variants and fusion proteins thereof. PD-L1 and PD-L2 polypeptides, fusion proteins, and soluble fragments can inhibit or reduce the inhibitory signal transduction that occurs through PD-1 in T cells by preventing endogenous ligands (i.e. endogenous PD-L1 and PD-L2) of PD-1 from interacting with PD-1. Additionally, the non-antibody agent may be soluble PD-1 fragments, PD-1 fusion proteins which bind to ligands of PD-1 and prevent binding to the endogenous PD-1 receptor on T cells. In one example, the PD-L2 fusion protein is B7-DC-Ig and the PD-1 fusion protein is PD-1-Ig. In another example, the PD-L1, PD-L2 soluble fragments are the extracellular domains of PD-L1 and PD-L2, respectively. In one embodiment, the payload of the conjugate may be a non-antibody agent disclosed in US publication No.: 2013/017199; the contents of which are incorporated by reference herein in its entirety.

In addition to CTLA-4 and PD-1, other known immune inhibitory checkpoints include TIM-3 (T cell immunoglobulin and mucin domain-containing molecule 3), LAG-3 (lymphocyte activation gene-3, also known as CD223), BTLA (B and T lymphocyte attenuator), CD200R, KRLG-1, 2B4 (CD244), CD160, MR (killer immunoglobulin receptor), TIGIT (T-cell immune-receptor with immunoglobulin and MM domains), VISTA (V-domain immunoglobulin suppressor of T-cell activation) and A2aR (A2a adenosine receptor) (Ngiow et al., Prospects for TIM3 targeted antitumor immunotherapy, Cancer Res., 2011, 71(21): 6567-6571; Liu et al., Immune-checkpoint proteins VISTA and PD-1 nonredundantly regulate murine T-cell responses, PNAS, 2015, 112(21): 6682-6687; and Baitsch et al., Extended Co-Expression of Inhibitory Receptors by Human CD8 T-Cells Depending on Differentiation, Antigen-Specificity and Anatomical Localization.2012, Plos One, 7(2): e30852). These molecules that similarly regulate T-cell activation are being assessed as targets of cancer immunotherapy.

TIM-3 is a transmembrane protein constitutively expressed on IFN-γ-secreting T-helper 1 (Th1/Tc1) cells (Monney et al., Th1-specific cell surface protein Tim-3 regulates macrophage activation and severity of an autoimmune disease. Nature. 2002, 415:536-541), DCs, monocytes, CD8⁺ T cells, and other lymphocyte subsets as well. TIM-3 is an inhibitory molecule that down-regulates effector Th1/Tc1 cell responses and induces cell death in Th1 cells by binding to its ligand Galectin-9, and also induces peripheral tolerance (Fourcade et al. Upregulation of Tim-3 and PD-1 expression is associated with tumor antigen-specific CD8+ T cell dysfunction in melanoma patients. J experimental medicine. 2010; 207:2175-2186). Blocking TIM-3 can enhance cancer vaccine efficacy (Lee et al., The inhibition of the T cell immunoglobulin and mucin domain 3(Tim-3) pathway enhances the efficacy of tumor vaccine. Biochem. Biophys. Res Commun, 2010, 402: 88-93).

It has been shown that extracellular adenosine generated from hypoxia in the tumor microenvironment binds to A2a receptor which is expressed on a variety of immune cells and endothelial cells. The activation of A2aR on immune cells induces increased production of immunosuppressive cytokines (e.g., TGF-β, IL-10), upregulation of alternate immune checkpoint pathway receptors (e.g., PD-1, LAG-3), increased FOXP3 expression in CD4+ T cells driving a regulatory T cell phenotype, and induction of effector T cell anergy. Beavis et al demonstrated that A2aR blockade can improve effector T cell function and suppress metastasis (Beavis et al., Blockade of A2A receptors potently suppresses the metastasis of CD73 + tumors. Proc Natl Acad Sci USA, 2013, 110: 14711-14716). Some A2aR inhibitors are used to block A2aR inhibitory signal, including, without limitation, SCH58261, SYN115, ZM241365 and FSPTP (Leone et al., A2aR antagonists: Next generation checkpoint blockade for cancer immunotherapy, Comput Struct Biotechnol. J 2015, 13: 265-272).

LAG-3 is a type I transmembrane protein expressed on activated CD4⁺ and CD8⁺ T cells, a subset of γδ T cells, NK cells and regulatory T cells (Tregs), and can negatively regulate immune response (Jha et al., Lymphocyte Activation Gene-3 (LAG-3) Negatively Regulates Environmentally-Induced Autoimmunity, PLos One, 2014, 9(8): e104484). LAG-3 negatively regulates T-cell expansion by inhibiting T cell receptor-induced calcium fluxes, thus controlling the size of the memory T-cell pool. LAG-3 signaling is important for CD4⁺ regulatory T-cell suppression of autoimmune responses, and LAG-3 maintains tolerance to self and tumor antigens via direct effects on CD8⁺ T cells. A recent study showed that blockade of both PD-1 and LAG-3 could provoke immune cell activation in a mouse model of autoimmunity, supporting that LAG-3 may be another important potential target for checkpoint blockade.

BTLA, a member of the Ig superfamily, binds to HVEM (herpesvirus entry mediator; also known as TNFRSF14 or CD270), a member of the tumor necrosis factor receptor superfamily (TNFRSF) (Watanabe et al., BTLA is a lymphocyte inhibitory receptor with similarities to CTLA-4 and PD-1 Nat Immunol, 2003, 4670-679. HVEM is expressed on T cells (e.g. CD8+ T cells). The HVEM-BTLA pathway plays an inhibitory role in regulating T cell proliferation (Wang et al., The role of herpesvirus entry mediator as a negative regulator of T cell-mediated responses, J Clin Invest., 2005, 115: 74-77). CD160 is another ligand of HVEM. The co-inhibitory signal of CD160/HVEM can inhibit the activation of CD4+ helper T cell (Cai et al., CD160 inhibits activation of human CD4⁻ T cells through interaction with herpesvirus entry mediator. Nat Immunol. 2008; 9:176-185).

CD200R is a receptor of CD200 that is expressed on myeloid cells. CD200 (OX2) is a highly expressed membrane glycoprotein on many cells. Studies indicated that CD200 and CD200R interaction can expand the myeloid-derived suppressor cell (MDSC) population (Holmannova et al., CD200/CD200R paired potent inhibitory molecules regulating immune and inflammatory responses; Part I: CD200/CD200R structure, activation, and function. Acta Medica (Hradec Kralove) 2012, 55(1):12-17; and Gorczynski, CD200 and its receptors as targets of immunoregulation, Curr Opin Investig Drug, 2005, 6(5): 483-488).

TIGIT is a co-inhibitory receptor that is highly expressed tumor-infiltrating T cells. In the tumor microenvironment, TIGIT can interact with CD226, a costimulatory molecule on T cells in cis, therefore disrupt CD226 dimerization. This inhibitory effect can critically limit antitumor and other CD8+ T cell-dependent responses (Johnston et al., The immunoreceptor TIGIT regulates antitumor and antiviral CD8(+) T cell effector function, Cancer cell, 2014, 26(6):923-937).

KIRs are a family of cell surface proteins expressed on natural killer cells (NKs). They regulate the killing function of these cells by interacting with MHC class I molecules expressed on any cell types, allowing the detection of virally infected cells or tumor cells. Most KIRs are inhibitory, meaning that their recognition of MHC molecules suppresses the cytotoxic activity of their NK cell (Ivarsson et al., Activating killer cell Ig-like receptor in health and disease, Frontier in Immu., 2014, 5: 1-9).

Additional coinhibitory signals that affect T cell activation include, but are not limited to KLRG-1, 2B4 (also called CD244), and VISTA (Lines et al., VISTA is a novel broad-spectrum negative checkpoint regulator for cancer immunotherapy, Cancer Immunol Res., 2014, 2(6): 510-517).

In accordance with the present invention, the payload of the conjugate may be an antagonist or inhibitor of a co-inhibitory molecule selected from CTLA-4, PD-1, PD-L1, PD-L2, TIM-3, LAG-3(CD223), BTLA, CD160, CD200R, TIGIT, KRLG-1, KIR, 2B4 (CD244), VISTA, A2aR and other immune checkpoints. In some aspects, the antagonist agent may be an antagonistic antibody, or a functional fragment thereof, against a coinhibitory checkpoint molecule selected from CTLA-4, PD-1, PD-L1, PD-L2, TIM-3, LAG-3(CD223), BTLA, CD160, CD200R, TIGIT, KRLG-1, KIR, 2B4 (CD244), VISTA and A2aR.

In some embodiments, the payload that is an antagonist or inhibitor of a co-inhibitory molecule selected from CTLA-4, PD-1, PD-L1, PD-L2, TIM-3, LAG-3(CD223), BTLA, CD160, CD200R, TIGIT, KRLG-1, KIR, 2B4 (CD244), VISTA, A2aR and other immune checkpoints may be conjugated to a cell penetrating peptide via a first cleavable linker, wherein the cell penetrating peptide is further conjugated to a chemotherapy agent or cytotoxic agent via a second cleavable linker. The payloads may act as a targeting moiety and target the conjugate to the immune checkpoints in tumor microenvironment. The cell penetrating peptide is capable of penetrating cell memberane. The cytotoxic agent is thereafter released to the tumor microenvironment and kills the tumor cells.

In some embodiments, the payload of the conjugate may be an antagonistic antibody, and/or a functional fragment thereof, specific to LAG-3(CD223). Such antagonistic antibodies can specifically bind to LAG-3(CD223) and inhibit regulatory T cells in tumors. In one example, it may be an antagonistic anti-LAG-3(CD223) antibody disclosed in U.S. Pat. Nos. 9,005,629 and 8,551,481. The payload may also be any inhibitor that binds to the amino acid motif KIEELE in the LAG-3(CD223) cytoplasmic domain which is essential for CD223 function, as identified using the methods disclosed in U.S. Pat. Nos. 9,005,629 and 8,551,481; the contents each of which are incorporated herein by reference in their entirety. Other antagonistic antibodies specific to LAG-3(CD223) may include antibodies disclosed in US publication NO.20130052642; the contents of which is incorporated herein by reference in its entirety.

In some embodiments, the payload of the conjugate may be an antagonistic antibody, and/or a functional fragment thereof, specific to TIM-3. Such antagonistic antibodies specifically bind to TIM-3 and can be internalized into TIM-3 expressed cells such as tumor cells to kill tumor cells. In other aspects, TIM-3 specific antibodies that specifically bind to the extracellular domain of TIM-3 can inhibit proliferation of TIM-3 expressing cells upon binding, e.g., compared to proliferation in the absence of the antibody and promote T-cell activation, effector function, or trafficking to a tumor site. In one example, the antagonistic anti-TIM-3 antibody may be selected from any antibody disclosed in U.S. Pat. Nos. 8,841,418; 8,709,412; 8,697,069; 8,647,623; 8,586,038; and 8,552,156; the contents of each of which are incorporated herein by reference in their entirety.

In addition, the antagonistic TIM-3 specific antibody may be monoclonal antibodies 8B.2C12, 25F.1D6 as disclosed in U.S. Pat. Nos. 8,697,069; 8,101,176; and 7,470,428; the contents of each of which are incorporated herein by reference in their entirety.

In other embodiments, the payload of the conjugate may be an agent that can specifically bind to galectin-9 and neutralize its binding to TIM-3, including neutralizing antibodies disclosed in PCT publication NO. 2015/013389; the contents of which are incorporated by reference in its entirety.

In some embodiments, the payload of the conjugate may be an antagonistic antibody, and/or a functional fragment thereof, specific to BTLA, including but not limited to antibodies and antigen binding portion of antibodies disclosed in U.S. Pat. Nos. 8,247,537; 8,580,259; fully human monoclonal antibodies in U.S. Pat. No.: 8,563,694; and BTLA blocking antibodies in U.S. Pat. No.: 8,188, 232; the contents of each of which are incorporated herein by reference in their entirety.

Other additional antagonist agents that can inhibit BTLA and its receptor HVEM may include agents disclosed in PCT publication NOs.: 2014/184360; 2014/183885; 2010/006071 and 2007/010692; the contents of each of which are incorporated herein by reference in their entirety.

In certain embodiments, the payload of the conjugate may be an antagonistic antibody, and/or a functional fragment thereof, specific to KIR, for example IPH2101 taught by Benson et al., (A phase I trial of the anti-KIR antibody IPH2101 and lenalidomide in patients with relapsed/refractory multiple myeloma, Clin Cancer Res., 2015, May 21. pii: clincanres.0304.2015); the contents of which are incorporated by reference in its entirety.

In other embodiments, the antagonist agent may be any compound that can inhibit the inhibitory function of a coinhibitory checkpoint molecule selected from CTLA-4, PD-1, PD-L1, PD-L2, TIM-3, LAG-3(CD223), BTLA, CD160, CD200R, TIGIT, KRLG-1, KIR, 2B4 (CD244), VISTA and A2aR.

In some examples, the antagonist agent may be a non-antibody inhibitor such as LAG-3-Ig fusion protein (IMP321) (Romano et al., J transl. Medicine, 2014, 12:97), and herpes simplex virus (HSV)-1 glycoprotein D (gD), an antagonist of BTLA)/CD160-HVEM) pathways (Lasaro et al., Mol Ther. 2011; 19(9): 1727-1736).

In some embodiments, the payload of the conjugate may be an agent that is bispecific or multiple specific. As used herein, the terms “bispecific agent” and “multiple specific agent” refer to any agent that can bind to two targets or multiple targets simultaneously. In some aspects, the bispecific agent may be a bispecific peptide agent that has a first peptide sequence that binds a first target and a second peptide sequence that binds a second different target. The two different targets may be two different inhibitory checkpoint molecules selected from CTLA-4, PD-1 PD-L1, PD-L2, TIM-3, LAG-3(CD223), BTLA, CD160, CD200R, TIGIT, KRLG-1, KIR, 2B4 (CD244), VISTA and A2aR. A non-limiting example of bispecific peptide agents is a bispecific antibody or antigen-binding fragment thereof. Similarly, a multiple specific agent may be a multiple peptide specific agent that has more than one specific binding sequence domain for binding to more than one target. For example, a multiple specific polypeptide can bind at least two, at least three, at least four, at least five, at least six, or more targets. A non-limiting example of multiple-specific peptide agents is a multiple-specific antibody or antigen-binding fragment thereof.

In one example, such bispecific agent is the bispecific polypeptide antibody variants for targeting TIM-3 and PD-1, as disclosed in US publication NO.: 2013/0156774; the content of which is incorporated herein by reference in its entirety.

In some embodiments, one, two or multiple checkpoint antagonists/inhibitors may be connected to the targeting moiety through the linker in one conjugate.

In other embodiments, the conjugate of the present invention may comprise two active agents that are connected to the targeting moiety through the linker, in which one active agent is an antagonist agent that specifically binds to an inhibitory molecule selected from CTLA-4, PD-1, PD-L1, PD-L2, TIM-3, LAG-3, BTLA, CD160, C200R, TIGIT, KLRG-1, KIR, 2B4/CD244, VISTA and Ara2R; the other active agent is an agonist agent that specifically binds to a stimulatory molecule selected from CD28, CD80(B7.1), CD86 (B7.2), 4-1BB(CD137), 4-1BBL (CD137L), CD27, CD70, CD40, CD40L, CD226, CD30, CD30L, OX40, OX40L, GITR and its ligand GITRL, LIGHT, LTβR, LTαβ, ICOS(CD278), ICOSL(B7-H2) and NKG2D.

### B. Targeting Regulatory Cells Unfiltrating the Tumor Microenvironment

Many regulatory cells with immunosuppressive potential can infiltrate the tumor microenvironment, including Regulatory T cells, microphages (M2) and MDSCs. Suppressive mechanisms employed by these cells involve secretion of cytokines (e.g., IL-10 and TGFβ), Growth factors (e.g., VEGF), secretion of enzymes (e.g., arginase, NOS and IDO), and expression of inhibitory receptors as discussed in the previous section(e.g., CTLA-4 and PD-L1). Depleting or modifying these regulatory cells and targeting each of the mechanisms they use within the tumor microenvironment can reverse immunosuppression.

Regulatory T cells (Tregs): Regulatory T cells (Tregs) have been widely recognized as crucial players in controlling immune responses. CD4+ regulatory T cells can constitutively express CD25 (IL-2 receptor α-chain) and the forkhead box P3 (FOXP3) transcription factor. CD25+ FOXP3+ and Type 1 regulatory T cells (Tr1) are induced in the thymus and IL-2 appears to be fundamental for their survival, expansion, and suppressive function. Activated CD4+CD25+FOXP3+ Tr1 cells can suppress CD4+ and CD8+ effector T cell proliferation and cytokine secretion, and inhibit B lymphocytes proliferation. Tr1 cells produce a large amount of IL-10 and TGF-β that inhibit Th1 and Th2 T cell responses. Tregs also maintain immune tolerance by restraining the activation, proliferation, and effector functions of natural killer (NK) and NKT cells, B cells and antigen presenting cells (APCs).

Depleting CD25+ regulatory T cells in the tumor microenvironment is a promising strategy for destructing cancer. Several studies showed that depletion of Treg cells using anti-CD25 antibody can enhance the efficacy of a variety of immunotherapies (Li et al., Complete regression of experimental solid tumors by combination LEC/chTNT-3 immunotherapy and CD25+ T-cell depletion. Cancer Res. 2003;63:8384-8392; Klages et al., Selective depletion of Foxp3+ regulatory T cells improves effective therapeutic vaccination against established melanoma. Cancer Res. 2010; 70:7788-7799).

In some embodiments, the payload of the conjugate may be an agent that can reduce or deplete regulatory T cell activity in tumors.

In one example, the agent for reducing or depleting regulatory T cell activity may be an antagonistic antibody that binds to CTLA-4, CD25, CD4, neuropillin. The antibody may be a full length antibody or a functional antibody fragment. The antibodies may include antibodies in U.S. Pat. No. 8,961,968; the contents of which are incorporated by reference in its entirety.

In one example, the agent for reducing or depleting regulatory T cell activity may include, but are not limited to, bivalent IL-2 fusion toxins as disclosed in PCT publication NO. 2014/093240; the contents of which are incorporated by reference herein in its entirety. The bivalent IL-2 fusion toxin comprises a cytotoxic protein (e.g., diphtheria toxin, pseudomonas exotoxin, or cytotoxic portions or variants thereof) fused with at least two Interleukin 2 (IL-2) sequences.

In one example, the agent for reducing or depleting regulatory T cell activity may be a neutralizing antibody that can block CCL-1(chemokine (C—C motif) ligand 1 (CCL1)); the neutralization of CCL-1 can deplete Treg cells and increase anti-cancer cells such as CD8+NKG2D+ T cells and NK cells (Hoelzinger et al., Blockade of CCL1 inhibits T regulatory cell suppressive function enhancing tumor immunity without affecting T effector responses. J Immunol. 2010; 184: 6833-6842).

In another example, the agent for reducing or depleting regulatory T cell activity may be a small molecule antagonist of CCR4. It has been shown that Treg recruitment to the tumor microenvironment can be blocked through neutralizing CCL17 and CCL22 using a small molecule antagonist of CCR4, which leads to improved responses to vaccine (CCR4 antagonist combined with vaccines induces antigen-specific CD8+ T cells and tumor immunity against self-antigens. Blood. 2011, 118: 4853-4862).

Myeloid-Derived Suppressor Cells (MDSCs): Myeloid-derived suppressor cells, which have immunosuppressive and pro-angiogenic activity, comprise a mixture of monocytes/macrophages, granulocytes, and dendritic cells (DCs) at different stages of differentiation. MDSCs maintain an immature phenotype when exposed to proinflammatory signals and contribute to a tumor-promoting type 2 phenotype by producing IL-10 and blocking macrophage to product IL-12. MDSCs inhibit the function of effector T cells and decrease NK cells cytotoxicity, cytokine production, and maturation of dendritic cells. It has also been suggested that MDSCs interact with Kuppfer cells to induce PD-L1 expression, which in turn inhibits antigen presentation.

MDSC differentiation can be blocked using cyclooxygenase (COX) inhibitors, which prevent the production of prostaglandin. All-trans retinoic acids (ATRA) have also been shown to reduce the presence of immature MDSC by converting them to non-immunosuppressive mature myeloid cells.

The chemokine CCL2 is an attractant for myeloid derived suppressor cells and its neutralization could augment the antitumor activity of vaccine or adoptive cytotoxic T lymphocytes (CTLs) transfer (Fridlender et al., CCL2 blockade augments cancer immunotherapy. Cancer Res. 2010; 70:109-118).

Monoclonal antibodies specific for GR-1 (Myeloid differentiation antigen, also known as Ly-6G) could deplete MDSCs and the depletion, when combined with adoptive T cell therapy can result in an enhancement of immunotherapy and regression of established tumors (Morales et al., Adoptive transfer of HER2/neu-specific T cells expanded with alternating gamma chain cytokines mediate tumor regression when combined with the depletion of myeloid-derived suppressor cells. Cancer. Immunol Immunother. 2009; 58:941-953)

In accordance with the present invention, the payload of the conjugate may be an agent that can deplete or reduce MDSCs in the tumor microenvironment. In some embodiments, the active agent may block differentiation and recruitment of MDSCs to the tumor sites. Such an agent may include but is not limited to, a cyclooxygenase (COX) inhibitor, a trans-retinoic acid, a neutralizing antibody specific to CCL-2, or a neutralizing antibody specific to GR-1. In one example, the agent that negative regulates MDSC may be a peptibody disclosed in PCT publication NO. 2015/048748; the contents of which are incorporated by reference in its entirety.

Regulatory DC cells: Tumor infiltrating regulatory DCs can suppress T-cell activation through IL-10 and indoleamine 2,3-dioxygenase (IDO) production. The immune tolerance effect contributes to immunosuppression in the tumor microenvironment (Holtzhausen et al., Melanoma-derived Wnt5a Promotes Local Dendritic-Cell Expression of IDO and Immunotolerance: Opportunities for Pharmacologic Enhancement of Immunotherapy. Cancer Immunol Res, 2015, Jun. 3. pii: canimm.0167.2014. [Epub ahead of print]).

Tumor infiltrating macrophages (TAMs): In most tumors, the infiltrated M2 microphages can secrete IL-10, TGF-β, and arginase, which provide an immunosuppressive microenvironment for tumor growth. Furthermore, tumor-associated M2 macrophages secrete many other cytokines, chemokines, and proteases, which promote tumor angiogenesis, growth, metastasis, and immunosuppression (Hao et al., Macrophages in Tumor Microenvironments and the Progression of Tumors, Clin Dev Immunol. 2012; 2012: 948098).

Clodronate encapsulated in liposomes is a reagent for the depletion of macrophages in vivo. This reagent can deplete M2 macrophages and increase the efficacy of therapies including anti-angiogenic therapy using anti-VEGF or agonist-CD137 and CpG combination immunotherapy (Zeisberger et al., Clodronate-liposome-mediated depletion of tumor-associated macrophages: a new and highly effective antiangiogenic therapy approach. Br J Cancer. 2006, 95:272-281).

Additionally, Macrophages possess a certain degree of plasticity with regard to phenotype, and it is possible to manipulate tumor-associated immunosuppressive M2 macrophages to become immuno-supportive M1 -like macrophage. Agonist anti-CD40 antibodies may be used to re-polarize macrophage in the tumor microenvironment (Buhtoiarov et al., Anti-tumor synergy of cytotoxic chemotherapy and anti-CD40 plus CpG-ODN immunotherapy through repolarization of tumor-associated macrophages. Immunology. 2011, 132: 226-239).

In accordance with the present invention, the payload of the conjugate may be an agent that can deplete or reduce tumor infiltrating macrophages (TAMs) activity. In some aspects, the agent for reducing or depleting TAM activity may include, but are not limited to, an anti-VEGF antibody and a functional antibody fragment thereof,

In accordance with the present invention, the payload of the conjugate may be an active agent that can block differentiation or recruitments of regulatory cells, or deplete regulatory cells, or reprogram immunosuppressive cells in the tumor microenvironments. It may be an antibody, polypeptide, a fusion protein and/or a small molecule.

In some embodiments, the active agent may be a targeted immunostimulatory antibody and fusion protein that inhibits the development or function of Tregs and MDSCs within the tumor microenvironment, therefore counteract or reverse immune tolerance of tumor cells. The targeted immunostimulatory antibody and fusion protein may bind an immunosuppressive cytokine and molecule expressed by Treg cells and MDSCs, such as CTLA-4/CD152, PD-L1/B7-1, TGF-β, RANKL (Receptor activator of nuclear factor-κB ligand), LAG-3, GITR/TNFRSF18 (glucocorticoid-induced tumor necrosis factor receptor family-related gene) and IL-10. Such conjugates contain a payload of an immunomodulatory moiety. Some of examples of such conjugates are discussed in U.S. Pat No. 8,993,524, which is incorporated herein by reference in its entirety, including a molecule that binds TGF-β and an extracellular ligand-binding domain of TGF-β receptor (e.g. TGF-βRII, TGF-βRIIb, or TGF-βRIII), which can inhibit the function of TGF-β. In other examples, the immunomodulatory moiety may be a molecule that specifically binds to RANKL, or an extracellular ligand-binding domain or ectodomain of RANK.

### C. Immunosuppressive Enzyme

The catabolism of the amino acids arginine and tryptophan has been associated with the immunosuppressive tumor microenvironment. Arginase (ARG) can deplete arginine, and indoleamine 2,3-dioxygenase (IDO) can degrade tryptophan present in the tumor microenvironment. Inhibitors that can block the activity of these enzymes may be used to enhance immunotherapy efficacy.

N-hydroxy-L-Arg (NOHA) used to target ARG-expressing M2 macrophages can increase the survival of sarcoma tumor bearing mice when combined with agonist OX40 therapy. Nitroaspirin or sildenafil (Viagra®), blocking ARG and nitric oxide synthase (NOS) simultaneously, could reduce function of MDSCs and increase the number of tumor infiltrating lymphocytes.

IDO inhibitors, such as 1-methyl-tryptophan, can improve various kinds of immunotherapies such as vaccines and adoptive T cell transfer. siRNA targeted to IDO, when loaded in DCs, can be directly used as cell vaccine (Zheng et al., Silencing IDO in dendritic cells: a novel approach to enhance cancer immunotherapy in s murine breast cancer model, Int. J Cancer, 2013, 132: 967-977)

### D. Chemokines, Cytokines and Other Soluble Factors Within the Tumor Microenvironment

Infiltrating regulatory cells and tumor cells secrete many chemokine, cytokines and growth factors to regulate the microenvironment. The cellular compositions in the tumor microenvironment are then further influenced by these factors. Infiltrating immune cells may be attracted in the responses to specific chemokines. Manipulating such profiles and their associated molecules in the tumor microenvironment can change the environment from immunosuppressive to immuno-potentiating with anti-cancer immunity.

As discussed above, IL-10 secreted by TAMs and tumor cells is an important immunosuppressive cytokine that favors tumor to escape from immune surveillance. IL-10 diminishes the production of inflammatory mediators and inhibits antigen presentation (Sabat et al., Biology of Interleukin 10, Cytokine Growth Factor Rev., 2010, 21:331-344).

Similarly, TGF-β in the tumor microenvironment can strengthen the immunosuppression through different mechanisms of inhibiting the cytolytic activity of NKG2D+natural killer (NK) cells, decreasing dendritic cells (DCs) migration and increasing apoptosis; and promoting tumor growth by the maintenance of Treg cell differentiation.

TGF-β inhibitors can be used to block TGF-β activity and lift immunosuppression, such as peptide inhibitors (Lopez et al., Peptide inhibitors of transforming growth factor beta enhance the efficacy of anti-tumor immunotherapy. Into J cancer, 2009, 125: 2614-2623).

VEGF is another tumor derived soluble factor that contributes to the immune tolerance in the tumor microenvironment by regulating dendritic cell (Johnson et al., Vascular endothelial growth factor and immunosuppression in cancer: current knowledge and potential for new therapy. 2007, Expert Opin Biol Ther., 7(4): 449-460).

Studies also showed that some chemokines are specific to tumors and changes to the microenvironment can increase efficacy of additional immunotherapy agents, for example, adoptive T cell transfer. CCL21-secreting tumors recruited more CD11b⁺CD11c⁻F4/80-Grl^{high}myeloid-derived suppressor cells (MDSCs) and regulatory T (Treg) cells (Shields, et al., Induction of lymphoidlike stroma and immune escape by tumors that express the chemokine CCL21, Science, 2010, 328:749-752).

Accordingly, in some embodiments of the present invention, the payload of the conjugate may be an antagonistic agent that binds specifically to a cytokine, a chemokine or a soluble factor that make a contribution to the immunosuppression in cancer, including those that are presently known and those yet to be identified as one of skill in the art will appreciate. In some aspects, the molecule may include, including IL-10, TGF-β, CCL-21, andVEGF. The antagonistic agent may be antibodies, functional antibody fragments, polypeptides, peptides, nucleic acids, aptamers, and small molecule compounds that bind specifically to the soluble factors. In some examples. The antagonistic agent may neutralize the activity of the targeted cytokine, chemokine, growth factor and other soluble factors.

### F. Other Tumor Associated Negative Factors

In addition to induce immunosuppressive TGF-β, PD-L1/B7-H1,VEGF and IL-10 to inhibit the differentiation and maturation of antigen-presenting dendritic cells and to promote the development of immunosuppressive CD4³⁰ regulatory T cells and MDSCs, in some cancers, particularly B cell cancers and B hematological malignancies, tumor cell also express HLA-G, a non-classical MHC class I human leukocyte antigen-G (HLA-G), which is a crucial tumor-driven immune escape molecule involved in immune tolerance. HLA-G and soluble counterparts are able to exert inhibitory functions by direct interactions with inhibitory receptors present on both innate cells such as natural killer cells, and adaptive immune cells as cytotoxic T and B lymphocytes. Another non-classical MHC molecule HLA-E is also reported recently in several human cancer types. HLA-E overexpression in tumor cells can restrain tumor specific cytotoxic T lymphocytes (Gooden et al., HLA-E expression by gynecological cancers restrains tumor-infiltrating CD8+ T lymphocytes, Proc Natl Acad Sci USA, 2011, 108(26): 10656-10661).

In some embodiments, the payload of the conjugate may be an antagonistic agent that can block HLA-G. The blocker may be soluble HLA-G peptides from US publication NO. 2011/0189238; the contents of which are incorporated herein by reference in its entirety. In other examples, the antagonistic agent may be antibodies and functional fragments thereof against the alpha3 domain of HLA-G protein as disclosed in PCT publication NO. 2014/072534; the contents of which are incorporated herein by reference in its entirety.

In some embodiments, the payload of the conjugate may be an antagonistic agent that can block HLA-E. In some examples, the antagonistic agent may be antibodies specific to the heavy chain of HLA-E disclosed in PCT publication NO. 2012/094252, and anti-HLA-E antibodies in PCT publication NO. 2014/008206; the contents of each of which are incorporated herein by reference in their entirety.

In some embodiments, the payload of the conjugate may be any molecule secreted by tumor cells including: growth factors, tumor antigens, cytokines, angiogenic factors, adhesion molecules, sialoproteins (e.g. osteopontin), integrins, carbohydrate structures, cell surface molecules, intra-cellular molecules, polynucleotides, oligonucleotides, proteins, peptides or receptors thereof. Secreted molecules such as, growth factors, cytokines and angiogenic factors comprise: VEGF, tumor necrosis factors (TNF) transforming growth factors (TGF), colony stimulating factors (CSF), Fibroblast growth factors (FGF), epidermal growth factor (EGF), platelet-derived growth factor (PDGF), interferons (IFN), interleukins, endostatins, osteopontin (bone sialoprotein (BSP)), or fragments thereof.

In some embodiments, the payload of the conjugate may comprise an active agent that is specific to other immune cell specific molecules that can modulate immune cell activity, including but not limited to, CD2, CD3, CD4, CD8a, CD11a, CD11b, CD11c, CD19, CD20, CD25 (IL-2Ra), CD26, CD44, CD54, CD56, CD62L (L-Selectin), CD69 (VEA), CD83, CD95 (Fas), TNFRSF14, ATAR, TR2, CD150 (SLAM), CD178 (FasL), CD209 (DC-SIGN), CD277, AITR, AITRL, HLA-A, HLA-B, HLA-C, HLA-D, HLA-R, HLA-Q, TCR-α, TCR-β, TCR-γ, TCR-δ, ZAP-70, NK1.1, T Cell receptor αβ (TCRαβ), T Cell receptor γδ (TCRγδ), T cell receptor ζ (TCRζ), TGFβRII, TNF receptor, CD1-339, Foxp3, mannose receptor, or DEC205, or variants thereof.

In some embodiments, the conjugate of the present invention may comprise two different payloads of which one agent is specific to a soluble factor in the tumor microenvironment such as IL-10, TGF-β, VEGF, CC chemokines such as CCL-21 and CCL-19, and the other active agent that is specific to a co-stimulatory molecule such as 4-1BB (CD137), 4-1BBL (CD137L), CD27, CD70, CD28, CD80 (B7-1), CD86 (B7-2), CD226, CD30 and CD30 ligand, CD40, CD154(CD40 ligand), GITR and GITR ligands, OX40 (CD134), OX40L, LIGHT, HVEM (CD270), NKG2D, RANK, LTβ (lymphotoxin receptor), LTαβ (ligand), or variants thereof.

In some embodiments, the conjugate of the present invention may comprise two different payloads of which one agent is specific to a soluble factor in the tumor microenvironment such as IL-10, TGF-β, VEGF, CC chemokines such as CCL-21 and CCL-19, and the other active agent that is specific to a co-inhibitory molecule such as CTLA-4 (CD152), PD-1(CD279), PD-L1 (B7-H1), PD-L2 (B7-DC), B7-H2 (ICOS), ICOSL (B7RP-1), B7-H3, B7-H4, TIM-3, LAG-3, BTLA, A2aR, CD200R, TIGIT, or variants thereof.

In some embodiments, the conjugate of the present invention may comprise two different payloads of which one agent is specific to a costimulatory molecule such as 4-1BB (CD137), 4-1BBL (CD137L), CD27, CD70, CD28, CD80 (B7-1), CD86 (B7-2), CD226, CD30 and CD30 ligand, CD40, CD154(CD40 ligand), GITR and GITR ligands, OX40 (CD134), OX40L, LIGHT, HVEM (CD270), NKG2D, RANK, LTβ (lymphotoxin receptor), LTαβ (ligand), or variants thereof, and the other active agent is specific to a co-inhibitory factor such as CTLA-4 (CD152), PD-1(CD279), PD-L1 (B7-H1), PD-L2 (B7-DC), B7-H2 (ICOS), ICOSL (B7RP-1), B7-H3, B7-H4, TIM-3, LAG-3, BTLA, A2aR, CD200R, TIGIT, or variants thereof.

In addition to antagonistic antibodies, the payloads of the conjugates that are specific to an immunoregulator may be aptamers, for example aptamer specifically binding to a soluble immunosuppressive factor and a co-modulating molecule. In one example, the aptamer may be a bispecific aptamer that binds to VEGF and 4-1BB, or a bispecific aptamer that binds to osteopontin and 4-1BB, as disclosed in US publication No. 2015/0086584; the content of which is incorporated by reference in its entirety.

### B. Linkers

The conjugates contain one or more linkers attaching the active agents and targeting moieties. The linker, Y, is bound to one or more active agents and a targeting ligand to form a conjugate, wherein the conjugate releases at least one active agent upon delivery to a target cell. The linker can be a C₁—C₁₀ straight chain alkyl, C₁—C₁₀ straight chain O-alkyl, C₁—C₁₀ straight chain substituted alkyl, C₁—C₁₀ straight chain substituted O-alkyl, C₄—C₁₃ branched chain alkyl, C₄—C₁₃ branched chain O-alkyl, C₂—C₁₂ straight chain alkenyl, C₂—C₁₂ straight chain O-alkenyl, C₃—C₁₂ straight chain substituted alkenyl, C₃—C₁₂ straight chain substituted O-alkenyl, polyethylene glycol, polylactic acid, polyglycolic acid, poly(lactide-co-glycolide), polycarprolactone, polycyanoacrylate, ketone, aryl, heterocyclic, succinic ester, amino acid, aromatic group, ether, crown ether, urea, thiourea, amide, purine, pyrimidine, bypiridine, indole derivative acting as a cross linker, chelator, aldehyde, ketone, bisamine, bis alcohol, heterocyclic ring structure, azirine, disulfide, thioether, hydrazone and combinations thereof. For example, the linker can be a C₃ straight chain alkyl or a ketone. The alkyl chain of the linker can be substituted with one or more substituents or heteroatoms. In some embodiments the linker contains one or more atoms or groups selected from —O—, —C(═O)—, —NR, —O—C(═O)—NR—, —S—, —S—S—. The linker may be selected from dicarboxylate derivatives of succinic acid, glutaric acid or diglycolic acid.

In some embodiments, the alkyl chain of the linker may optionally be interrupted by one or more atoms or groups selected from —O—, —C(═O)—, —NR, —O—C(═O)—NR—, —S—, —S—S—. The linker may be selected from dicarboxylate derivatives of succinic acid, glutaric acid or diglycolic acid.

In some embodiments, the linker may be cleavable and is cleaved to release the active agent. In one embodiment, the linker may be cleaved by an enzyme. As a non-limiting example, the linker may be a polypeptide moiety, e.g. AA in WO2010093395 to Govindan, the content of which is incorporated herein by reference in its entirety; that is cleavable by intracellular peptidase. Govindan teaches AA in the linker may be a di, tri, or tetrapeptide such as Ala-Leu, Leu-Ala-Leu, and Ala-Leu-Ala-Leu. In another example, the cleavable linker may be a branched peptide. The branched peptide linker may comprise two or more amino acid moieties that provide an enzyme cleavage site. Any branched peptide linker disclosed in WO1998019705 to Dubowchik, the content of which is incorporated herein by reference in its entirety, may be used as a linker in the conjugate of the present invention. As another example, the linker may comprise a lysosomally cleavable polypeptide disclosed in U.S. Pat. No. 8,877,901 to Govindan et al., the content of which is incorporated herein by reference in its entirety. As another example, the linker may comprise a protein peptide sequence which is selectively enzymatically cleavable by tumor associated proteases, such as any Y and Z structures disclosed in U.S. Pat. No. 6,214,345 to Firestone et al., the content of which is incorporated herein by reference in its entirety.

In one embodiment, the cleaving of the linker is non-enzymatic. Any linker disclosed in US 20110053848 to Cleemann et al., the contents of which are incorporated herein by reference in their entirety, may be used. For example, the linker may be a non-biologically active linker represented by formula (I).

In one embodiment, the linker may be a beta-glucuronide linker disclosed in US 20140031535 to Jeffrey, the contents of which are incorporated herein by reference in their entirety. In another embodiment, the linker may be a self-stabilizing linker such as a succinimide ring, a maleimide ring, a hydrolyzed succinimide ring or a hydrolyzed maleimide ring, disclosed in US20130309256 to Lyon et al., the contents of which are incorporated herein by reference in their entirety. In another embodiment, the linker may be a human serum albumin (HAS) linker disclosed in US 20120003221 to McDonagh et al., the contents of which are incorporated herein by reference in their entirety. In another embodiment, the linker may comprise a fullerene, e.g., C₆₀, as disclosed in US 20040241173 to Wilson et al., the contents of which are incorporated herein by reference in their entirety. In another embodiment, the linker may be a recombinant albumin fused with polycysteine peptide as disclosed in U.S. Pat. No. 8,541,378 to Ahn et al., the contents of which are incorporated herein by reference in their entirety. In another embodiment, the linker comprises a heterocycle ring. For example, the linker may be any heterocyclic 1,3-substituted five- or six-member ring, such as thiazolidine, disclosed in US 20130309257 to Giulio, the content of which is incorporated herein by reference in its entirety.

In some embodiments, the linker may be used with compositions of the invention are well known in the art, and include, e.g., thyroglobulin, albumins such as human serum albumin, tetanus toxoid, polyamino acid residues such as poly L-lysine, poly L-glutamic acid, influenza virus proteins, hepatitis B virus core protein, and the like.

In some embodiments, the linker may be a hydrophilic linker as disclosed by Zhao et al. in PCT patent publication NO., WO2014/080251; the content of which is incorporated by reference in its entirety. The hydrophilic linkers may contain phosphinate, sulfonyl, and/or sulfoxide groups to link active agents (payloads) to a cell-targeting moiety.

In other embodiments, the linker promotes cellular internalization. In certain embodiments, the linker promotes cellular internalization. A variety of linkers that can be used with the present compositions and methods are described in WO 2004/010957, US2012/0141509, and US2012/0288512, which are incorporated by reference herein in their entirety.

In some embodiments, the linker of the conjugate may be optional. In this context, the active agent and the targeting moiety of the conjugate are directly connected to each other.

### C. Targeting Moieties

In some cases, the targeting moiety can also act as a therapeutic agent.

In some embodiments, the targeting moiety does not substantially interfere with efficacy of the therapeutic agent in vivo.

In accordance with the present invention, a conjugate can contain one or more targeting moieties or targeting ligands. For example, the conjugate can include an active agent with multiple targeting moieties each attached via a different linker. The conjugate can have the structure X—Y—Z—Y—X where each X is a targeting moiety that may be the same or different, each Y is a linker that may be the same or different, and Z is the active agent (payload).

Targeting ligands or moieties can be polypeptides (e.g., antibodies), peptides, antibody mimetics, nucleic acids (e.g., aptamers), glycoproteins, small molecules, carbohydrates, lipids, nanoparticles.

One barrier in developing cancer vaccine using tumor specific antigens is the less effective delivery of antigens to the antigen presenting cells (APCs). Increasing delivery of tumor specific antigens can enhance antigen presentation. In one embodiment, a targeting moiety may particularly target a conjugate of the present invention to an immune cell, a tumor cell or a location where an anti-cancer immune response occurs.

In some embodiments, the targeting moiety does not substantially interfere with efficacy of the therapeutic agent in vivo. In some cases, the targeting moiety itself can be an active agent. In other aspects, the targeting moiety may contain adjuvant activity, in addition to targeted binding to a cell of interest.

In some embodiments, the targeting moiety, X, may be a peptide such as a TAA peptide epitope (e.g., an amino acid sequence motif) that can specifically bind to a MHC/HLA protein (HLA class I or class II). Peptide antigens can be attached to MHC class I/II molecules by affinity binding within the cytoplasm before they are presented on the cell surface. The affinity of an individual peptide antigen is directly linked to its amino acid sequence and the presence of specific binding motifs in defined positions within the amino acid sequence. Such defined amino acid motifs may be used as targeting moieties.

In some embodiments, the targeting moiety, X, may be other peptides such as somatostatin, octeotide, LHRH (luteinizing hormone releasing hormone), epidermal growth factor receptor (EGFR) binding peptide, aptide or bipodal peptide, RGD-containing peptides, a protein scaffold such as a fibronectin domain, a single domain antibody, a stable scFv, or other homing peptides.

As non-limiting examples, a protein or peptide based targeting moiety may be a protein such as thrombospondin, tumor necrosis factors (TNF), annexin V, an interferon, angiostatin, endostatin, cytokine, transferrin, GM-CSF (granulocyte-macrophage colony-stimulating factor), or growth factors such as vascular endothelial growth factor (VEGF), hepatocyte growth factor (HGF), (platelet-derived growth factor (PDGF), basic fibroblast growth factor (bFGF), and epidermal growth factor (EGF).

In some embodiments, the targeting moiety is an antibody, an antibody fragment, RGD peptide, folic acid or prostate specific membrane antigen (PSMA). In some embodiments, the protein scaffold may be an antibody-derived protein scaffold. Non-limiting examples include single domain antibody (dAbs), nanobody, single-chain variable fragment (scFv), antigen-binding fragment (Fab), Avibody, minibody, CH2D domain, Fcab, and bispecific T-cell engager (BiTE) molecules. In some embodiments, scFv is a stable scFv, wherein the scFv has hyperstable properties. In some embodiments, the nanobody may be derived from the single variable domain (VHH) of camelidae antibody.

In some embodiments, the targeting moiety is a tumor cell binding moiety. For example, it may bind to a somatostatin receptor (SSTR) such as SSTR2 on tumor cells or luteinizing hormone releasing hormone receptor (LHRHR or GNRHR) such as GNRHR1 on tumor cells.

In some embodiments, the tumor cell binding moiety binds to to a cell surface protein selected from the group consisting of CD20, carcinoembryonic antigen (CEA), epithelial cell adhesion molecule (EpCAM), and CD19. Non-limiting examples of CD19 binding agents that may be used as a tumor cell binding moiety in the conjugates include any CD19 binding agent disclosed in Dreier et al. (J Immunol., vol. 170:4397 (2003)), in Klinger et al. (Blood, vol. 119:6226 (2012)), or blinatumomab, a bispecific single-chain antibody targeting CD3 and CD19 antigen disclosed in Topp et al. (J Clin Oncol., vol. 29:2493 (2011)). Non-limiting examples of CD20 binding agents include anti-CD20/CD3 T cell-dependent bispecific antibody disclosed in Sun et al. (Sci Transl Med., vol. 7:287 (2015)) or anti-CD3×anti-CD20 bispecific antibody disclosed in Gall et al. (Exp Hematol., vol. 33(4):452 (2005)). Non-limiting examples of CEA binding agents include CEA/CD3-bispecific T cell-engaging (BiTE) antibody disclosed in Osada et al. (Cancer Immunol Immunother., vol. 64(6):677 (2015)). Non-limiting examples of EpCAM binding agents include EpCAM/CD3-bispecific T-cell engaging antibody MT110 disclosed in Cioffi et al. (Clin. Cancer Res., vol. 18(2):465 (2012)).

In some embodiments, the tumor cell binding moiety is a protein scaffold. The protein scaffold may be a non-antibody-derived protein scaffold, wherein the protein scaffold is based on nonantibody binding proteins. The protein scaffold may be based on engineered Kunitz domains of human serine protease inhibitors (e.g., LAC1-D1), DARPins (designed ankyrin repeat domains), avimers created from multimerized low-density lipoprotein receptor class A (LDLR-A), anticalins derived from lipocalins, knottins constructed from cysteine-rich knottin peptides, affibodies that are based on the Z-domain of staphylococcal protein A, adnectins or monobodies and pronectins based on the 10^{th} or 14^{th} extracellular domain of human fibronectin III, Fynomers derived from SH3 domains of human Fyn tyrosine kinase, or nanofitins (formerly Affitins) derived from the DNA binding protein Sac7d.

In some embodiments, the protein scaffold may be based on a fibronectin domain. In some embodiments, the protein scaffold may be based on fibronectin type III (FN3) repeat protein. In some embodiments, the protein scaffold may be based on a consensus sequence of multiple FN3 domains from human Tenascin-C (hereinafter “Tenascin”). Any protein scaffold based on a fibronectin domain disclosed in U.S. Pat. No. 8,569,227 to Jacobs et al., the content of which is incorporated herein by reference in its entirety; may be used as a targeting moiety of the conjugate of the invention.

In some embodiments, the protein scaffold may be any protein scaffold disclosed in Mintz and Crea, BioProcess, vol. 11(2):40-48 (2013), the contents of which are incorporated herein by reference in their entirety. Any of the protein scaffolds disclosed in Tables 2-4 of Mintz and Crea may be used as a targeting moiety of the conjugate of the invention.

In some embodiments, the targeting moiety is an arginylglycylaspartic acid (RGD) peptide, a tripeptide composed of L-arginine, glucine and L-aspartic acid, which is a common cell targeting element for cellular attachment via integrins.

In some embodiments, a targeting moiety may be an antibody that specifically binds to a TAA and/or an antigenic peptide (epitope). As one skilled in the art can envision, an antibody fragment (e.g., an Fc fragment of an antibody) may be used for the same purpose.

In addition to tumor cells specific antigen or antigen epitopes, antibodies may be specific to a ubiquitous antigenic site on various cancers. Many studies have revealed that cancer cells share certain common characteristics. Many types of human cancer cells are characterized by substantial abnormalities in the glycosylation patterns of their cell-surface proteins and lipids (e.g., Hakomori et. al., 1996, Cancer Res. 56:5309-18; and Springer et al., 1997, J Mol Med 75:594-602). These differences have led to the identification of antigenic determinants on cancer cells. Natural IgM antibodies to these epitopes are present in the circulation and can be used as a targeting moiety of a conjugate of the present invention.

As non-limiting examples, the antibody targeting moiety may be connected to one or more components of the complement system (or other cytotoxic agents) to induce complement mediated tumor cell lysis. In this context, a conjugate may have a formula of (one or more cytotoxic agents)-linker—mAb.

In some embodiments, the targeting moiety is an antibody mimetic such as a monobody, e.g., an ADNECTIN™ (Bristol-Myers Squibb, New York, N.Y.), an Affibody® (Affibody AB, Stockholm, Sweden), Affilin, nanofitin (affitin, such as those described in WO 2012/085861, an AnticalinTM, an avimers (avidity multimers), a DARPin™, a Fynomer™, Centyrin™, and a Kunitz domain peptide. In certain cases, such mimetics are artificial peptides or proteins with a molar mass of about 3 to 20 kDa. Nucleic acids and small molecules may be antibody mimetic.

In some embodiments, the targeting moiety X may be an aptide or bipodal peptide. X may be any D-Aptamer-Like Peptide (D-Aptide) or retro-inverso Aptide which specifically binds to a target comprising: (a) a structure stabilizing region comprising parallel, antiparallel or parallel and antiparallel D-amino acid strands with interstrand noncovalent bonds; and (b) a target binding region I and a target binding region II comprising randomly selected n and m D-amino acids, respectively, and coupled to both ends of the structure stabilizing region, as disclosed in US Pat. Application No. 20140296479 to Jon et al., the content of which is incorporated herein by reference in its entirety. X may be any bipodal peptide binder (BPB) comprising a structure stabilizing region of parallel or antiparallel amino acid strands or a combination of these strands to induce interstrand non-covalent bonds, and target binding regions I and II, each binding to each of both termini of the structure stabilizing region, as disclosed in US Pat. Application No. 20120321697 to Jon et al., the content of which is incorporated herein by reference in its entirety. X may be an intracellular targeting bipodal-peptide binder specifically binding to an intracellular target molecule, comprising: (a) a structure-stabilizing region comprising a parallel amino acid strand, an antiparallel amino acid strand or parallel and antiparallel amino acid strands to induce interstrand non-covalent bonds; (b) target binding regions I and II each binding to each of both termini of the structure-stabilizing region, wherein the number of amino acid residues of the target binding region I is n and the number of amino acid residues of the target binding region II is m; and (c) a cell-penetrating peptide (CPP) linked to the structure-stabilizing region, the target binding region I or the target binding region II, as disclosed in US Pat. Application No. 20120309934 to Jon et al., the content of which is incorporated herein by reference in its entirety. X may be any bipodal peptide binder comprising a β-hairpin motif or a leucine-zipper motif as a structure stabilizing region comprising two parallel amino acid strands or two antiparallel amino acid strands, and a target binding region I linked to one terminus of the first of the strands of the structure stabilizing region, and a target binding region II linked to the terminus of the second of the strands of the structure stabilizing region, as disclosed in US Pat. Application No. 20110152500 to Jon et al., the content of which is incorporated herein by reference in its entirety. X may be any bipodal peptide binder targeting KPI as disclosed in WO2014017743 to Jon et al, any bipodal peptide binder targeting cytokine as disclosed in WO2011132939 to Jon et al., any bipodal peptide binder targeting transcription factor as disclosed in WO201132941 to Jon et al., any bipodal peptide binder targeting G protein-coupled receptor as disclosed in WO2011132938 to Jon et al., any bipodal peptide binder targeting receptor tyrosine kinase as disclosed in WO2011132940 to Jon et al., the content of each of which is incorporated herein by reference in their entirety. X may also be bipodal peptide binders targeting cluster differentiation (CD7) or an ion channel.

In some embodiments, the targeting moiety is a stabilized peptide. Intramolecular crosslinkers are used to maintain the peptide in the desired configuration, for example using disulfide bonds, amide bonds, or carbon-carbon bonds to link amino acid side chains. Such peptides which are conformationally stabilized by means of intramolecular cross-linkers are sometimes referred to as “stapled” peptides. The cross-linkers connect at least two amino acids of the peptide. The cross-linkers may comprise at least 5, 6, 7, 8, 9, 10, 11, or 12 consecutive carbon-carbon bonds. The cross-linkers may comprise at least 5, 6, 7, 8, 9, 10, 11, or 12 carbon atoms. Stapled peptides may penetrate cell membranes and bind to an intracellular receptor.

In one non-limiting example, the stapled peptide is a cross-linked alpha-helical polypeptide comprising a crosslinker wherein a hydrogen atom attached to an a-carbon atom of an amino acid of the peptide is replaced with a substituent of formula R—, wherein R— is alkyl, alkenyl, alkynyl, arylalkyl, cycloalkylalkyl, heteroalkyl, or heterocycloalkyl, unsubstituted or substituted with halo-, as disclosed in US 20140323701 to Nash et al., the contents of which are incorporated herein by reference in their entirety. In another example, the stapled peptides have improved in vivo half life such as any stapled peptide disclosed in US 20100298201 to Nash et al., the contents of which are incorporated herein by reference in their entirety. In another example, the tumor cell binding moiety may be any stapled peptide disclosed in U.S. Pat. No. 9,175,045 to Nash et al., the contents of which are incorporated herein by reference in their entirety, wherein the stapled peptide possesses reduced affinity to serum proteins while still remaining sufficient affinity to cell membranes. In another example, the cross-linker of the stapled peptide links the a-positions of at least two amino acids, such as any stapled peptide disclosed in U.S. Pat. No. 9,175,047 to Nash et al., the contents of which are incorporated herein by reference in their entirety. In another example, the tumor cell binding moiety comprise any stapled peptide disclosed in U.S. Pat. No. 8,927,500 to Guerlavais et al., the contents of which are incorporated herein by reference in their entirety, wherein the stapled peptide has homology to p53 protein and can bind to the MDM2 and/or MDMX proteins. In another example, the stapled peptide generates a reduced antibody response. Any stapled peptide disclosed in U.S. Pat. No. 8,808,694 to Nash et al., the contents of which are incorporated herein by reference in their entirety, may be used as a tumor cell binding moiety. In another example, the staped peptide may be any polypeptide with optimized protease stability disclosed in US 20110223149 to Nash et al., the contents of which are incorporated herein by reference in their entirety.

In some embodiments, the targeting moiety is a nanofintin® (also known as affinity) (Affilogic). Nanofitin, as used as herein, refers to a single-chain antibody mimic that are much smaller than antibodies. Nanofitins are small and stable, lack disulfide bridges, and can be produced at high levels. The molecular weight of nanofitins are below 10 KDa, preferably around 7 KDa. Because of their small size and short half-life, nanofitins may both accumulate specifically at the site of the tumor and be cleared from the serum rapidly, therefore reducing off-target toxicity compared to long lasting antibodies. Conjugates comprise nanofitins may deliver an active agent deeper into a tumor. Nanofitins may bind intracellular targets and affect intracellular protein-protein interaction.

In certain embodiments, the targeting moiety may be a bispecific T-cell engagers, an aptamer such as RNA, DNA or an artificial nucleic acid; a small molecule; a carbohydrate such as mannose, galactose or arabinose; a lipid, a vitamin such as ascorbic acid, niacin, pantothenic acid, carnitine, inositol, pyridoxal, lipoic acid, folic acid (folate), riboflavin, biotin, vitamin B12, vitamin A, E, and K.

In some embodiments, the targeting moiety may comprise a nucleic acid targeting moiety. In general, a nucleic acid targeting moiety is any nucleic acid that binds to an organ, tissue, cell, or a component associated therewith such as extracellular matrix component, and intracellular compartment. In some embodiments, the targeting moiety may be an aptamer, which is generally an oligonucleotide (e.g., DNA, RNA, or an analog or derivative thereof) that binds to a particular target, such as a polypeptide. In one embodiment, the targeting moiety may be an aptamer that targets to an immune cell (e.g., dendritic cells). Aptamers may be generated from libraries of single-stranded nucleic acids against different molecules via CELL-SELEX method in which whole living cells (e.g., dendritic cells) are used as targets for the aptamers (Ganji et al., Aptamers: new arrows to target dendritic cells, J Drug Target. 2015, 7: 1-12).

In some embodiments, the targeting moiety may be a non-immunoreactive ligand. For example, the non-immunoreactive ligand may be insulin, insulin-like growth factors I and II, lectins, apoprotein from low density lipoprotein, etc. as disclosed in US 20140031535 to Jeffrey, the content of which is incorporated herein by reference in its entirety. Any protein or peptide comprising a lectin disclosed in WO2013181454 to Radin, the content of which is incorporated herein by reference in its entirety, may be used as a targeting moiety.

In some embodiments, targeting moieties may be Lymph Node-targeting nanoparticle (NP)-conjugates (Jeanbart et al., Enhancing efficacy of anticancer vaccines by targeted delivery to tumor-draining lymph nodes. Cancer Immunol Res., 2014, 2(5): 436-437; the content of which is incorporated by reference in its entirety.

In some embodiments, the conjugate may have a terminal half-life of longer than about 72 hours and a targeting moiety may be selected from Table 1 or 2 of US 20130165389 to Schellenberger et al., the contents of which are incorporated herein by reference in their entirety. The targeting moiety may be an antibody targeting delta-like protein 3 (DLL3) in disease tissues such as lung cancer, pancreatic cancer, skin cancer, etc., as disclosed in WO2014125273 to Hudson, the contents of which are incorporated herein by reference in their entirety. The targeting moiety may also any targeting moiety in WO2007137170 to Smith, the contents of which are incorporated herein by reference in their entirety. The targeting moiety binds to glypican-3 (GPC-3) and directs the conjugate to cells expressing GPC-3, such as hepatocellular carcinoma cells.

In some embodiments, the targeting moiety may be a modified viral surface protein or fragments thereof.

In some embodiments, the targeting moiety may be an antigen recognition domain/sequence of TCR molecules. The nature of antigen recognition of such moieties will bind to an antigen-MHC molecule complex on the surface of cells, therefore deliver an active payload linked to the targeting moieties through a linker in the conjugate to the tumor cells.

In some embodiments, targeting moieties may be derived from the binding domains of the MHC class I and II molecules, for example, the a3 domain of the a chain of the MHC class I molecule. The α3 domain in the MHC class I molecule can specifically bind to CD8 on T cells, and the binding between CD8 and the α3 domain may deliver tumor antigen payloads near to the surface of T cells and activate TCR to bind the tumor antigens. In another example, the targeting moiety may be the β2 domain of the MHC class II molecules.

In some embodiments, the targeting moiety may be a cell binding element such as a ligand which binds to a cell surface receptor. In specific embodiments, the cell binding element may be selected from the group consisting of a Fc fragment, a toxin cell binding domain, a cytokine, a chemokine, a small peptide and an antibody. In some examples, the cytokines, chemokines and other immunomodulatory molecules are ligands of cell receptors on certain types of immune cells such as APCs (e.g., DCs), T cells, B cells, NK cells and macrophages.

In some embodiments, targeting moieties may be used to deliver antigens to APCs (Frenz et al., Antigen presenting cell selective drug delivery by glycan-decorated nanocarriers. Eur J Pharm Biopharm, 2015, Feb. 19, pii: S0939-6411), such as DEC-205 antibody as targeting moieties for targeted delivery of antigens to APCs.

In some embodiments, the targeting moiety binds to a receptor on T cells. In one embodiment, the targeting moiety binds to a checkpoint receptor such as CTLA-4 or PD-1 on T cells. Any peptide, antibody, antagonist, or a functional fragment thereof that binds to CTLA-4 or PD-1 discussed in “Checkpoint inhibitors” section may be used as a targeting moiety. In one embodiment, the targeting moiety is a peptide comprising between 5 and 50 amino acids, between 10 and 40 amino acids, or between 20 and 30 amino acids. In another embodiment, the targeting moiety does not inhibit the function of T cells. In yet another embodiment, the targeting moiety acts as an inhibitor of CTLA-1 and/or PD-1, wherein the binding of CTLA-4 ligands to CTLA-4 and/or PD-1 ligands (such as PD-L1 and PD-L2) to PD-1 is blocked. In these embodiments, the active agent may be any active agent disclosed in copending PCT/US2015/038562, the contents of which are incorporated herein by reference in their entirety, such as anti-cancer agents including but not limited to DNA-binding or alkylating drugs, doxorubicin or analogs, CC-1065 or analogs, calicheamicins, microtubule stabilizing and destabilizing agents, maytansinoids or analogs, auristatins, tubulysin compounds, vinca alkaloids, epothilone compounds, cryptophycin compounds, platinum compounds, topoisomerase I inhibitors, and so on.

In some embodiments, the conjugate of the present invention may comprise a targeting moiety that specifically targets to a regulatory immune cell, an effector immune cell, and/or a tumor cell. The regulatory immune cells may be immune cells that infiltrate the tumor site, including regulatory T cells, MDSCs, regulatory DCs and TAMs. The Effector cell may be a CD4+ T helper cell, a CD8+ T cell, a B cell, a NK cell, or any other effector immune cells.

In some examples, the targeting moiety may target to a regulatory T cell by targeting to a T cell specific molecule such as CD4, CD25, CTLA-4, VEGF, FOXP3 and other regulatory T cell specific markers identified in U.S. Pat. No. 9,040,051; the contents of which are incorporated by reference in its entirety.

In some examples, the targeting moiety may target to a myeloid derived suppressor cell by targeting to a MDSC cell specific molecule such as CD15; IL4Ra; CD14; CD11b; HLA-DR; CD33; Lin; FSC; SSC; and, optionally CD45; CD18; CD80; CD83; CD86; HLA-I; a Live/Dead discriminator.

In some examples, the targeting moiety targets to a tumor infiltrating macrophage by targeting to a tumor infiltrating macrophage specific molecule.

In other examples, the targeting moiety of the conjugate may target to an immune cell by targeting to any one of the immune cell marker selected from HLA-DR, CD30, CD33, CD52, MUC1, TAC, carbonic anhydrase IX, B7, CCCL19, CCCL21, CSAp, CD1, CD1a, CD2, CD3, CD4, CDS, CD6, CD7, CD8, CD11A, CD11B, CD11C, CD11D, CD14, CD15, CD16, CD18, CD19, CD20, CD21, CD22, CD23, CD24, CD25, CD29, CD30, CD32b, CD33, CD37, CD38, CD40, CD40L, CD44, CD45, CD46, CD47, CD52, CD54, CD55, CD56, CD59, CD64, CD66, CD67, CD70, CD74, CD79a, CD80, CD83, CD95, CD126, CD133, CD138, CD140A, CD140B, CD147, CD149, CD154,CD210, CD215, CD270, CD307a, CD307b, CD307c, CD307d, CD307e, CD351, Cd352, CD353, CD354, CD355, CD357, CD358, CD360, Cd361, CD362, CD363, CD364, CEACAMS, CEACAM-6, CCR2, CCR3, CCR4, CCR5, CCR7, CCR8, CCR9, CXCR4, CXCR6, Galectin-3, alpha-fetoprotein (AFP), BLR1, ED-B fibronectin, EGP-1, EGP-2, EGF receptor (ErbB1), ErbB2, ErbB3, ENPP3, Factor H, FHL-1, Flt-3, folate receptor, Ga 733, GROB, HMGB-1, hypoxia inducible factor (HIF), HM1.24, ILGF, IFN-γ, IFN-α, IFN-β, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, IL*RA, IL8RB, MIF, MUC1, MUC2, MUC3, MUC4, MUC5, VEGFR-1, VEGFR-2, VEGFR-3, RANTES, T101, TEK, TRAILR-1, TRAILR-2 and complement factors C3, C3a, C3b, C5a, C5.

In some embodiments, the targeting moiety includes an antibody, antibody fragment, scFv, Fv, dsFv, ds-scFV, Fd, linear antibody, minibody, diabody, bibody, tribody, scdiabody, kappabody, BiTE, DVD-Ig, SIP, SMIP, DART, an antibody analogue comprising one or more CDRs, or Fc-containing polypeptide that specifically binds a component of a tumor cell, tumor antigen, tumor vasculature, tumor microenvironment, or tumor-infiltrating immune cells. The selection of an antibody as the targeting moiety may be based on its specificity to an antigen expressed on a target cell or at a target site, of interest.

In some embodiments, targeting moieties may be a single-chain antibody mimic that are much smaller than antibodies such as nanofintin® (also known as affinity) (Affilogic) disclosed in copending U.S. Application No. 62/308,908, or peptides which are conformationally stabilized by means of intramolecular cross-linkers referred to as “stapled” peptides disclosed in copending U.S. Application No. 62/291,212, the contents of each of which are incorporated herein by reference in their entirety.

Masked Targeting Moiety Complex

In some embodiments, the targeting moiety may be a targeting moiety complex comprising a target binding moiety (TBM) and a masking moiety (MM). In some embodiments, MM may be attached to TBM directly, via a non-cleavable moiety, or via a cleavable moiety (CM). In some other embodiments, MM is bound to the payload or the linker of the conjugate directly, via a non-cleavable moiety, or via a cleavable moiety (CM).

TBM may be any targeting moiety discussed above including small molecules, peptides or derivatives, an antibody or a fragment thereof. In some embodiments, TBM may be a peptide comprising between 5 to 50 amino acids, between 10 to 40 amino acids, or between 20 to 30 amino acids. In some embodiments, TBM may be small molecules.

The binding of TBM to its target is inhibited or hindered by MM. For example, the binding may be sterically hindered by the presence of MM or may be inhibited by the charge of MM. Leaving of MM upon cleavage of CM, a conformation change, or a chemical transformation may unmask TBM. The masking/unmasking process may be reversible or irreversible.

In one example wherein TBM is attached to MM with a CM, TBM might be less accessible to its target when CM is uncleaved. Upon cleavage of CM, MM no longer interferes with the binding of the targeting moiety to its target, thereby activating the conjugates of the present invention. The cleavable moiety prevents binding of the conjugates of the present invention at nontreatment sites. Such conjugates can further provide improved biodistribution characteristics.

MM may be selected from a plurality of polypeptides based on its ability to inhibit binding of the TBM to the target in an uncleaved state and allow binding of the TBM to the target in a cleaved state.

CM may locate between TBM and MM in the targeting moiety complex, or may locate within MM. CM may be cleaved by an enzyme such as protease. CM may comprise a peptide that may be a substrate for an enzyme selected from the group consisting of MMP1, MMP2, MMP3, MMP8, MMP9, MMP14, plasmin, PSA, PSMA, CATHEPSIN D, CATHEPSIN K, CATHEPSIN S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE. For example, CM may comprise a protease substrate such as a plasmin substrate, a caspase substrate or a matrix metalloprotease (MMP) substrate (e.g., a substrate of MMP-1, MMP-2, MMP-9, or MMP-14). Alternatively, CM may be cleaved by a reducing agent capable of reducing a disulfide bond between a cysteine-cysteine pair. CM may comprise a cysteine-cysteine pair capable of forming a reducible disulfide bond. Reducing agents of particular interest include cellular reducing agents such as proteins or other agents that are capable of reducing a disulfide bond under physiological conditions, e.g., glutathione, thioredoxin, NADPH, flavins, and ascorbate.

In one example, the targeting moiety complex may be any activatable binding polypeptides (ABPs) disclosed in U.S. Pat. No. 9,169,321 to Daugherty et al. (CytomX), the contents of which are incorporated herein by reference in their entirety. For example, the targeting moiety complex may be an enzyme activatable binding polypeptide (ABP) that binds CTLA-4, VEGF, or VCAM-1. In other examples, the the targeting moiety complex may be an activatable binding polypeptide (ABP) that binds epidermal growth factor disclosed in U.S. Pat. No. 9,120,853 to Lowman et al., an ABP that binds Jagged 1 or Jagged 2 disclosed in U.S. Pat. No. 9,127,053 to West et al., activatable anti-CD3 antibodies disclosedin WO2016014974 to Irving et al., activatable antibodies that bind to interleukin-6 receptor (IL6R) disclosed in WO2014052462 to West et al., activatable proproteins disclosed in US20150203559 to Stagliano et al., any modified antibody or activatable antibody disclosed in US20140024810 to Stagliano et al., WO2015089283 to Desnoyers et al., WO2015066279 to Lowman et al., WO2015048329 to Moore et al., US20150079088 to Lowman et al., WO2014197612 to Konradi et al., US20140023664 to Lowman et al., the contents of each of which are incorporated herein by reference in their entirety.

In some embodiments, the targeting moiety may be a targeting moiety complex comprising a target binding moiety (TBM) and a photocleavable moiety. The binding of TBM to its target is reversibly inhibite by the photocleavable moiety. TBM may be any targeting moiety discussed above including small molecules, peptides or derivatives, an antibody or a fragment thereof. A “photocleavable moiety” means any agent attached to the antibody which can be removed on exposure to electromagnetic energy such as light energy of any desired variety whether visible, UV, X-ray or the like (e g microwave). The photocleavable moiety may be a reagent which couples to hydroxy or amino residues present in TBM. Thus phosgene, diphosgene, DCCI or the like may be used to generate photocleavable esters, amides, carbonates and the like from a wide range of alcohols. For example, substituted arylalkanols are employed, particularly nitorphenyl methyl alcohol, 1-nitrophenylethan-1-ol and substituted analogues. The photocleavable moiety may be located at or about the binding site of TBM.

In one example, the targeting moiety complex may comprise any photocleavable moiety disclosed in WO1996034892 to Self et al., the contents of which are incorporated herein by reference in their entirety. TBM may be an antibody component that retain the active site and bind to a tumor cell marker. TBM may also be any antibody component made against suitable cells such as T-cells, cytotoxic T-cell clones, cytotoxic T-cells and activated peripheral blood lymphocytes, CD3+ lymphocytes, CD16+ lymphocytes, Fc gamma R111, the low affinity Fc gamma receptor for polymorphonuclear leucocytes, macrophages and large granular lymphocytes, B-lymphocyte markers, myeloid cells, T Lymphocyte CD2, CD3, CD4, CD8, dengue virus, lymphokine activated killer (LAK) cells, NK cells or monocytes. TBM may be a monoclonal antibody anti-CD-3 OKT3 against T-cells, or a monoclonal antibody that binds to tumor antigen carcinoembrionic antigen (CEA).

In certain embodiments, the targeting moiety or moieties of the conjugate are present at a predetermined molar weight percentage from about 1% to about 10%, or about 10% to about 20%, or about 20% to about 30%, or about 30% to about 40%, or about 40% to about 50%, or about 50% to about 60%, or about 60% to about 70%, or about 70% to about 80%, or about 80% to about 90%, or about 90% to about 99% such that the sum of the molar weight percentages of the components of the conjugate is 100%. The amount of targeting moieties of the conjugate may also be expressed in terms of proportion to the active agent(s), for example, in a ratio of ligand to active agent of about 10:1, 9:1, 8:1, 7:1, 6:1, 5:1, 4:1, 3:1, 2:1, 1:1, 1:2, 1:3, 1:4; 1:5, 1:6, 1:7, 1:8, 1:9, or 1:10.

### D. Pharmacokinetic Modulating Unit

The conjugates of the present invention may further comprise at least one external linker connected to a reacting group that reacts with a functional group on a protein or an engineered protein or derivatives/analogs/mimics thereof, or comprise at least one external linker connected to a pharmacokinetic modulating unit. The external linkers connecting the conjugates and the reacting group or the pharmacokinetic modulating units may be cleavable linkers that allow release of the conjugates. Hence, the conjugates may be separated from the protein or pharmacokinetic modulating units as needed.

In some embodiments, the conjugates comprise at least one reacting group that reacts with a functional group on a protein or an engineered protein or derivatives/analogs/mimics thereof. The reaction between the reacting group and the functional group may happen in vivo after administration or is performed prior to administration. The protein may be a naturally occurring protein such as a serum or plasma protein, or a fragment thereof. Particular examples include thyroxine-binding protein, transthyretin, α1-acid glycoprotein (AAG), transferrin, fibrinogen, albumin, an immunoglobulin, α-2-macroglobulin, a lipoprotein, or fragments thereof. The reaction between the reacting group and the functional group may be reversible.

In one example, the functional group is on human serum albumin (HSA or albumin) or its derivative/analog/mimic. Albumin is the most abundant plasma protein (35-50 g/L in human serum) with a molecular weight of 66.5 KDa and an effective diameter of 7.2 nm (Kratz, J. of Controlled Release, vol. 132:171, (2008), the contents of which are incorporated herein by reference in their entirety). Albumin has a half-life of about 19 days. Albumin preferentially accumulates in malignant and inflamed tissues due to a leaky capillary and an absent or defective lymphatic drainage system. Albumin accumulates in tumors such as solid tumors also because albumin is a major energy and nutrition source for turmor growth. The function group may be the cysteine-34 position of albumin that has an accessible free thiol group. Reacting groups that react with a functional group on albumin or it derivative/analog/mimic may be selected from a disulfide group, a vinylcarbonyl group, a vinyl acetylene group, an aziridine group, an acetylene group or any of the following groups:

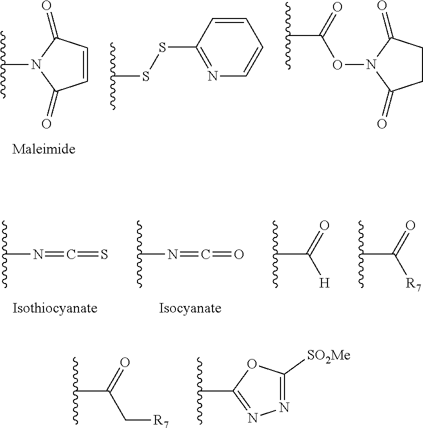

where R⁷ is Cl, Br, F, mesylate, tosylate, O-(4-nitrophenyl), O-pentafluorophenyl, and wherein optionally the activated disulfide group, the vinylcarbonyl group, the vinyl acetylene group, the aziridine group, and the acetylene group may be substituted. The reacting group may also be any protein-binding moiety disclosed in U.S. Pat. No. 9,216,228 to Kratz et al., the contents of which are incorporated herein by reference in their entirety, selected from the group consisting of a maleinimide group, a halogenacetamide group, a halogenacetate group, a pyridylthio group, a vinylcarbonyl group, an aziridine group, a disulfide group, a substituted or unsubstituted acetylene group, and a hydroxysuccinimide ester group. In some cases, the reacting group is a disulfide group. The disulfide group undergoes an exchange with a thiol group on a protein or an engineered protein or a polymer or derivatives/analogs/mimics thereof, such as albumin, to form a disulfide between the conjugate and the protein or an engineered protein or a polymer or derivatives/analogs/mimics thereof.

In another example, the functional group is on transthyretin or its derivative/analog/mimic. Transthyretin is a 55 KDa serum protein that has an in vivo half-life of around 48 h. Reacting groups that react with a functional group on transthyretin or it derivative/analog/mimic may be selected from AG10 (structure shown below) or its derivative disclosed by Penchala et al. in Nature Chemical Biology, vol. 11:793, (2015) or formula (I), (II), (III) or (IV) (structures shown below) disclosed in U.S. Pat. No. 5,714,142 to Blaney et al., the contents of each of which are incorporated herein by reference in their entirety. Any transthyretin-selective ligand disclosed on pages 5-8 of Blaney et al. or their derivatives may be used as a reacting group, such as but not limited to, tetraiodothyroacetic acid, 2,4,6-triiodophenol, flufenamic acid, diflunisal, milrinone, EMD 21388.

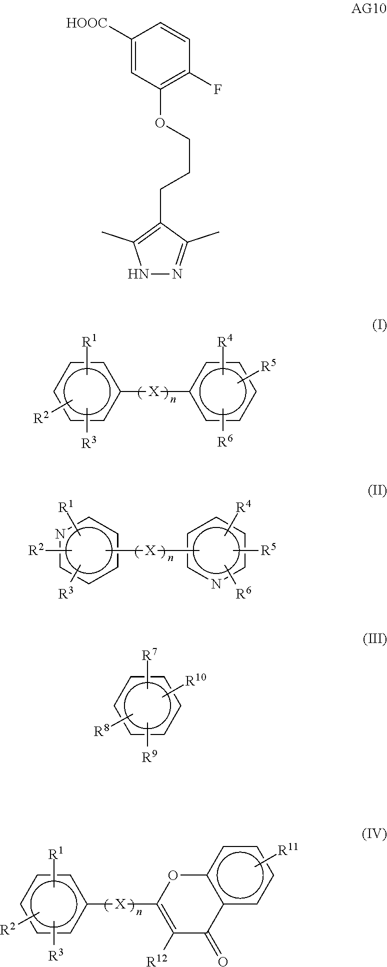

In some cases, the reacting group may be any protein binding moiety may be any protein binding moiety disclosed in U.S. Pat. No. 9,216,228 to Kratz, the contents of which are incorporated herein by reference in their entirety, such as a maleimide group, a halogenacetamide group, a halogenacetate group, a pyridylthio group, a vinylcarbonyl group, an aziridin group, a disulfide group, a substituted or unsubstituted acetylene group, and a hydroxysuccinimide ester group.

In some embodiments, the conjugates comprise at least one pharamacokinetic modulating unit. The pharmacokinetic modulating unit may be a natural or synthetic protein or fragment thereof. For example, it may be a serum protein such as thyroxine-binding protein, transthyretin, al-acid glycoprotein (AAG), transferrin, fibrinogen, albumin, an immunoglobulin, α-2-macroglobulin, a lipoprotein, or fragments thereof. The pharmacokinetic modulating unit may also be a natural or synthetic polymer, such as polysialic acid unit, a hydroxyethyl starch (HES) unit, or a polyethylene glycol (PEG) unit. Further, the pharmacokinetic modulating unit may be a particle, such as dendrimers, inorganic nanoparticles, organic nanoparticles, and liposomes.

The pharmacokinetic modulating unit or pharmacokinetic modulating units have a total molecular weight of at least about 10 KDa, at least about 20 KDa, at least about 30 KDa, at least about 40 KDa or at least about 50 KDa. Generally, the pharmacokinetic modulating unit or pharmacokinetic modulating units have a total molecular weight between about 10 KDa and about 70 KDa. Preferably, the pharmacokinetic modulating unit or pharmacokinetic modulating units have a total molecular weight between about 30 KDa and about 70 KDa, between about 40 KDa and about 70 KDa, between about 50 KDa and about 70 KDa, between about 60 KDa and about 70 KDa.

### II. Particles and Nanoparticles

Particles comprising one or more conjugates can be polymeric particles, lipid particles, solid lipid particles, inorganic particles, or combinations thereof (e.g., lipid stabilized polymeric particles). In some embodiments, the conjugates are substantially encapsulated or particularly encapsulated in the particles. In some embodiments, the conjugates are disposed on the surface of the particles. The conjugates may be attached to the surface of the particles with covalent bonds, or non-covalent interactions. In some embodiments, the conjugates of the present invention self-assemble into a particle.

As used herein, the term “encapsulate” means to enclose, surround or encase. As it relates to the formulation of the conjugates of the invention, encapsulation may be substantial, complete or partial. The term “substantially encapsulated” means that at least greater than 50, 60, 70, 80, 85, 90, 95, 96, 97, 98, 99, 99.9, 99.99 or greater than 99.999% of conjugate of the invention may be enclosed, surrounded or encased within the particle. “Partially encapsulation” means that less than 10, 10, 20, 30, 40 50 or less of the conjugate of the invention may be enclosed, surrounded or encased within the particle. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the particle. Encapsulation may be determined by any known method. In some embodiments, the particles are polymeric particles or contain a polymeric matrix. The particles can contain any of the polymers described herein or derivatives or copolymers thereof. The particles will generally contain one or more biocompatible polymers. The polymers can be biodegradable polymers. The polymers can be hydrophobic polymers, hydrophilic polymers, or amphiphilic polymers. In some embodiments, the particles contain one or more polymers having an additional targeting moiety attached thereto. In some embodiments, the particles are inorganic particles, such as but not limited to, gold nanoparticles and iron oxide nanoparticles.

The size of the particles can be adjusted for the intended application. The particles can be nanoparticles or microparticles. The particle can have a diameter of about 10 nm to about 10 microns, about 10 nm to about 1 micron, about 10 nm to about 500 nm, about 20 nm to about 500 nm, or about 25 nm to about 250 nm. In some embodiments the particle is a nanoparticle having a diameter from about 25 nm to about 250 nm. In some embodiments, the particle is a nanoparticle having a diameter from about 50 nm to about 150 nm. In some embodiments, the particle is a nanoparticle having a diameter from about 70 nm to about 130 nm. In some embodiments, the particle is a nanoparticle having a diameter of about 100 nm. It is understood by those in the art that a plurality of particles will have a range of sizes and the diameter is understood to be the median diameter of the particle size distribution. Polydispersity index (PDI) of the particles may be ≤about 0.5, ≤about 0.2, or ≤about 0.1. Drug loading may be ≥about 1%, ≥about 5%, ≥about 10%, or ≥out 20%. Drug loading, as used herein, refers to the weight ratio of the conjugates of the invention and depends on maximum tolerated dose (MTD) of free drug conjugate. Particle ζ-potential (in 1/10^{th} PBS) may be ≤0 mV or from about −10 to 0 mV. Drug released in vitro from the particle at 2 h may be less than about 60%, less than about 40%, or less than about 20%. Regarding pharmacokinetics, plasma area under the curve (AUC) in a plot of concentration of drug in blood plasma against time may be at least 2 fold greater than free drug conjugate, at least 4 fold greater than free drug conjugate, at least 5 fold greater than free drug conjugate, at least 8 fold greater than free drug conjugate, or at least 10 fold greater than free drug conjugate. Tumor PK/PD of the particle may be at least 5 fold greater than free drug conjugate, at least 8 fold greater than free drug conjugate, at least 10 fold greater than free drug conjugate, or at least 15 fold greater than free drug conjugate. The ratio of Cₘₐₓ of the particle to Cₘₐₓ of free drug conjugate may be at least about 2, at least about 4, at least about 5, or at least about 10. Cₘₐₓ, as used herein, refers to the maximum or peak serum concentration that a drug achieves in a specified compartment or test area of the body after the drug has been administrated and prior to the administration of a second dose. The ratio of MTD of a particle to MTD of free drug conjugate may be at least about 0.5, at least about 1, at least about 2, or at least about 5. Efficacy in tumor models, e.g., TGI %, of a particle is better than free drug conjugate. Toxicity of a particle is lower than free drug conjugate.

In various embodiments, a particle may be a nanoparticle, i.e., the particle has a characteristic dimension of less than about 1 micrometer, where the characteristic dimension of a particle is the diameter of a perfect sphere having the same volume as the particle. The plurality of particles can be characterized by an average diameter (e.g., the average diameter for the plurality of particles). In some embodiments, the diameter of the particles may have a Gaussian-type distribution. In some embodiments, the plurality of particles have an average diameter of less than about 300 nm, less than about 250 nm, less than about 200 nm, less than about 150 nm, less than about 100 nm, less than about 50 nm, less than about 30 nm, less than about 10 nm, less than about 3 nm, or less than about 1 nm. In some embodiments, the particles have an average diameter of at least about 5 nm, at least about 10 nm, at least about 30 nm, at least about 50 nm, at least about 100 nm, at least about 150 nm, or greater. In certain embodiments, the plurality of the particles have an average diameter of about 10 nm, about 25 nm, about 50 nm, about 100 nm, about 150 nm, about 200 nm, about 250 nm, about 300 nm, about 500 nm, or the like. In some embodiments, the plurality of particles have an average diameter between about 10 nm and about 500 nm, between about 50 nm and about 400 nm, between about 100 nm and about 300 nm, between about 150 nm and about 250 nm, between about 175 nm and about 225 nm, or the like. In some embodiments, the plurality of particles have an average diameter between about 10 nm and about 500 nm, between about 20 nm and about 400 nm, between about 30 nm and about 300 nm, between about 40 nm and about 200 nm, between about 50 nm and about 175 nm, between about 60 nm and about 150 nm, between about 70 nm and about 130 nm, or the like. For example, the average diameter can be between about 70 nm and 130 nm. In some embodiments, the plurality of particles have an average diameter between about 20 nm and about 220 nm, between about 30 nm and about 200 nm, between about 40 nm and about 180 nm, between about 50 nm and about 170 nm, between about 60 nm and about 150 nm, or between about 70 nm and about 130 nm. In one embodiment, the particles have a size of 40 to 120 nm with a zeta potential close to 0 mV at low to zero ionic strengths (1 to 10 mM), with zeta potential values between +5 to −5 mV, and a zero/neutral or a small −ve surface charge.

APCs such as macrophages are good at phagocytosis and may be stimulated by nanoparticles. The active agents of the conjugates in the nanoparticle are then released inside the APCs. In some embodiments, the active agents are only released within certain environments, such as with the presence of lysozymes. In some embodiments, particles, nanoparticles and/or polymerica nanoparticles target bone marrow and delivers conjugates to bone marrow.

In some embodiments, the particles of the invention may comprise more than one conjugates. The conjugates may be different, e.g., comprising different payloads. In some embodiments, the particles of the invention may comprises conjugates having different PK values. Conjugates in the same particle are protected by the particle and are released at the same time. In some embodiments, linkers of the conjugates are cleaved under the same condition and payloads of the conjugates are released at the same time. In some embodiments, linkers of the conjugates are cleaved under different conditions and payloads of the conjugates are released sequentially.

In some embodiments, the weight percentage of the conjugate in the particles is at least about 0.05%, 0.1%, 0.5%, 1%, 5%, 10%, 15%, 20%, 25%, 30%, 35%, 40%, 45%, or 50% such that the sum of the weight percentages of the components of the particles is 100%. In some embodiments, the weight percentage of the conjugate in the particles is from about 0.5% to about 10%, or about 10% to about 20%, or about 20% to about 30%, or about 30% to about 40%, or about 40% to about 50%, or about 50% to about 60%, or about 60% to about 70%, or about 70% to about 80%, or about 80% to about 90%, or about 90% to about 99% such that the sum of the weight percentages of the components of the particles is 100%.

### A. Polymers

The particles may contain one or more polymers. Polymers may contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide, collectively referred to herein as “PLA”, and caprolactone units, such as poly(c-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker.

The particles may contain one or more hydrophilic polymers. Hydrophilic polymers include cellulosic polymers such as starch and polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol); polyoxazoline; and copolymers thereof.

The particles may contain one or more hydrophobic polymers. Examples of suitable hydrophobic polymers include polyhydroxyacids such as poly(lactic acid), poly(glycolic acid), and poly(lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof.

In certain embodiments, the hydrophobic polymer is an aliphatic polyester. In some embodiments, the hydrophobic polymer is poly(lactic acid), poly(glycolic acid), or poly(lactic acid-co-glycolic acid).

The particles can contain one or more biodegradable polymers. Biodegradable polymers can include polymers that are insoluble or sparingly soluble in water that are converted chemically or enzymatically in the body into water-soluble materials. Biodegradable polymers can include soluble polymers crosslinked by hydolyzable cross-linking groups to render the crosslinked polymer insoluble or sparingly soluble in water.

Biodegradable polymers in the particle can include polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose such as methyl cellulose and ethyl cellulose, hydroxyalkyl celluloses such as hydroxypropyl cellulose, hydroxy-propyl methyl cellulose, and hydroxybutyl methyl cellulose, cellulose ethers, cellulose esters, nitro celluloses, cellulose acetate, cellulose propionate, cellulose acetate butyrate, cellulose acetate phthalate, carboxylethyl cellulose, cellulose triacetate, cellulose sulphate sodium salt, polymers of acrylic and methacrylic esters such as poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene imines), poly(caprolactones), poly(hydroxyalkanoates), poly(hydroxyvalerates), polyanhydrides, poly(acrylic acids), polyglycolides, poly(urethanes), polycarbonates, polyphosphate esters, polyphosphazenes, derivatives thereof, linear and branched copolymers and block copolymers thereof, and blends thereof. In some embodiments the particle contains biodegradable polyesters or polyanhydrides such as poly(lactic acid), poly(glycolic acid), and poly(lactic-co-glycolic acid).

The particles can contain one or more amphiphilic polymers. Amphiphilic polymers can be polymers containing a hydrophobic polymer block and a hydrophilic polymer block. The hydrophobic polymer block can contain one or more of the hydrophobic polymers above or a derivative or copolymer thereof. The hydrophilic polymer block can contain one or more of the hydrophilic polymers above or a derivative or copolymer thereof. In some embodiments the amphiphilic polymer is a di-block polymer containing a hydrophobic end formed from a hydrophobic polymer and a hydrophilic end formed of a hydrophilic polymer. In some embodiments, a moiety can be attached to the hydrophobic end, to the hydrophilic end, or both. The particle can contain two or more amphiphilic polymers. B. Lipids

The particles may contain one or more lipids or amphiphilic compounds. For example, the particles can be liposomes, lipid micelles, solid lipid particles, or lipid-stabilized polymeric particles. The lipid particle can be made from one or a mixture of different lipids. Lipid particles are formed from one or more lipids, which can be neutral, anionic, or cationic at physiologic pH. The lipid particle is preferably made from one or more biocompatible lipids. The lipid particles may be formed from a combination of more than one lipid, for example, a charged lipid may be combined with a lipid that is non-ionic or uncharged at physiological pH.

The particle can be a lipid micelle. Lipid micelles for drug delivery are known in the art. Lipid micelles can be formed, for instance, as a water-in-oil emulsion with a lipid surfactant. An emulsion is a blend of two immiscible phases wherein a surfactant is added to stabilize the dispersed droplets. In some embodiments the lipid micelle is a microemulsion. A microemulsion is a thermodynamically stable system composed of at least water, oil and a lipid surfactant producing a transparent and thermodynamically stable system whose droplet size is less than 1 micron, from about 10 nm to about 500 nm, or from about 10 nm to about 250 nm. Lipid micelles are generally useful for encapsulating hydrophobic active agents, including hydrophobic therapeutic agents, hydrophobic prophylactic agents, or hydrophobic diagnostic agents.

The particle can be a liposome. Liposomes are small vesicles composed of an aqueous medium surrounded by lipids arranged in spherical bilayers. Liposomes can be classified as small unilamellar vesicles, large unilamellar vesicles, or multi-lamellar vesicles. Multi-lamellar liposomes contain multiple concentric lipid bilayers. Liposomes can be used to encapsulate agents, by trapping hydrophilic agents in the aqueous interior or between bilayers, or by trapping hydrophobic agents within the bilayer.

The lipid micelles and liposomes typically have an aqueous center. The aqueous center can contain water or a mixture of water and alcohol. Suitable alcohols include, but are not limited to, methanol, ethanol, propanol, (such as isopropanol), butanol (such as n-butanol, isobutanol, sec-butanol, tert-butanol, pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

The particle can be a solid lipid particle. Solid lipid particles present an alternative to the colloidal micelles and liposomes. Solid lipid particles are typically submicron in size, i.e. from about 10 nm to about 1 micron, from 10 nm to about 500 nm, or from 10 nm to about 250 nm. Solid lipid particles are formed of lipids that are solids at room temperature. They are derived from oil-in-water emulsions, by replacing the liquid oil by a solid lipid.

Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids.

Suitable cationic lipids include, but are not limited to, N-[1-(2,3-dioleoyloxy)propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]—N,N-dimethyl amine(DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N-(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N,N,N′,N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyhalkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

Suitable solid lipids include, but are not limited to, higher saturated alcohols, higher fatty acids, sphingolipids, synthetic esters, and mono-, di-, and triglycerides of higher saturated fatty acids. Solid lipids can include aliphatic alcohols having 10-40, preferably 12-30 carbon atoms, such as cetostearyl alcohol. Solid lipids can include higher fatty acids of 10-40, preferably 12-30 carbon atoms, such as stearic acid, palmitic acid, decanoic acid, and behenic acid. Solid lipids can include glycerides, including monoglycerides, diglycerides, and triglycerides, of higher saturated fatty acids having 10-40, preferably 12-30 carbon atoms, such as glyceryl monostearate, glycerol behenate, glycerol palmitostearate, glycerol trilaurate, tricaprin, trilaurin, trimyristin, tripalmitin, tristearin, and hydrogenated castor oil. Suitable solid lipids can include cetyl palmitate, beeswax, or cyclodextrin.

Amphiphilic compounds include, but are not limited to, phospholipids, such as 1,2 distearoyl-sn-glycero-3-phosphoethanolamine (DSPE), dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of between 0.01-60 (weight lipid/w polymer), for example, between 0.1-30 (weight lipid/w polymer). Phospholipids which may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcho-line (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophos-phoethanolamine. Synthetic phospholipids with asymmetric acyl chains (e.g., with one acyl chain of 6 carbons and another acyl chain of 12 carbons) may also be used.

### C. Hydrophobic Ion-Pairing Complexes

The particles may comprise hydrophobic ion-pairing complexes or hydrophobic ioin-pairs formed by one or more conjugates described above and counterions.

Hydrophobic ion-pairing (HIP) is the interaction between a pair of oppositely charged ions held together by Coulombic attraction. HIP, as used here in, refers to the interaction between the conjugate of the present invention and its counterions, wherein the counterion is not H⁺ or HO⁻ ions. Hydrophobic ion-pairing complex or hydrophobic ion-pair, as used herein, refers to the complex formed by the conjugate of the present invention and its counterions. In some embodiments, the counterions are hydrophobic. In some embodiments, the counterions are provided by a hydrophobic acid or a salt of a hydrophobic acid. In some embodiments, the counterions are provided by bile acids or salts, fatty acids or salts, lipids, or amino acids. In some embodiments, the counterions are negatively charged (anionic). Non-limited examples of negative charged counterions include the counterions sodium sulfosuccinate (AOT), sodium oleate, sodium dodecyl sulfate (SDS), human serum albumin (HSA), dextran sulphate, sodium deoxycholate, sodium cholate, anionic lipids, amino acids, or any combination thereof. Non-limited examples of positively charged counterions include 1,2-dioleoyl-3-trimethylammonium-propane (chloride salt) (DOTAP), cetrimonium bromide (CTAB), quaternary ammonium salt didodecyl dimethylammonium bromide (DMAB) or Didodecyldimethylammonium bromide (DDAB). Without wishing to be bound by any theory, in some embodiments, HIP may increase the hydrophobicity and/or lipophilicity of the conjugate of the present invention. In some embodiments, increasing the hydrophobicity and/or lipophilicity of the conjugate of the present invention may be beneficial for particle formulations and may provide higher solubility of the conjugate of the present invention in organic solvents. Without wishing to be bound by any theory, it is believed that particle formulations that include HIP pairs have improved formulation properties, such as drug loading and/or release profile. Without wishing to be bound by any theory, in some embodiments, slow release of the conjugate of the invention from the particles may occur, due to a decrease in the conjugate's solubility in aqueous solution. In addition, without wishing to be bound by any theory, complexing the conjugate with large hydrophobic counterions may slow diffusion of the conjugate within a polymeric matrix. In some emobodiments, HIP occurs without covalent conjugation of the counterion to the conjugate of the present invention.

Without wishing to be bound by any theory, the strength of HIP may impact the drug load and release rate of the particles of the invention. In some embodiments, the strength of the HIP may be increased by increasing the magnitude of the difference between the pKa of the conjugate of the present invention and the pKa of the agent providing the counterion. Also without wishing to be bound by any theory, the conditions for ion pair formation may impact the drug load and release rate of the particles of the invention.

In some embodiments, any suitable hydrophobic acid or a combination thereof may form a HIP pair with the conjugate of the present invention. In some embodiments, the hydrophobic acid may be a carboxylic acid (such as but not limited to a monocarboxylic acid, dicarboxylic acid, tricarboxylic acid), a sulfinic acid, a sulfenic acid, or a sulfonic acid. In some embodiments, a salt of a suitable hydrophobic acid or a combination thereof may be used to form a HIP pair with the conjugate of the present invention. Examples of hydrophobic acids, saturated fatty acids, unsaturated fatty acids, aromatic acids, bile acid, polyelectrolyte, their dissociation constant in water (pKa) and logP values were disclosed in WO2014/043,625, the content of which is incorporated herein by reference in its entirety. The strength of the hydrophobic acid, the difference between the pKa of the hydrophobic acid and the pKa of the conjugate of the present invention, logP of the hydrophobic acid, the phase transition temperature of the hydrophobic acid, the molar ratio of the hydrophobic acid to the conjugate of the present invention, and the concentration of the hydrophobic acid were also disclosed in WO2014/043,625, the content of which is incorporated herein by reference in its entirety.

In some embodiments, particles of the present invention comprising a HIP complex and/or prepared by a process that provides a counterion to form HIP complex with the conjugate may have a higher drug loading than particles without a HIP complex or prepared by a process that does not provide any counterion to form HIP complex with the conjugate. In some embodiments, drug loading may increase 50%, 100%, 2 times, 3 times, 4 times, 5 times, 6 times, 7 times, 8 times, 9 times, or 10 times.

In some embodiments, the particles of the invention may retain the conjugate for at least about 1 minute, at least about 15 minutes, at least about 1 hour, when placed in a phosphate buffer solution at 37° C.

### D. Additional Active Agents

The particles can contain one or more additional active agents in addition to those in the conjugates. The additional active agents can be therapeutic, prophylactic, diagnostic, or nutritional agents as listed above. The additional active agents can be present in any amount, e.g. from about 1% to about 90%, from about 1% to about 50%, from about 1% to about 25%, from about 1% to about 20%, from about 1% to about 10%, or from about 5% to about 10% (w/w) based upon the weight of the particle. In one embodiment, the agents are incorporated in a about 1% to about 10% loading w/w.

### E. Additional Targeting Moieties

The particles can contain one or more targeting moieties targeting the particle to a specific organ, tissue, cell type, or subcellular compartment in addition to the targeting moieties of the conjugate. The additional targeting moieties can be present on the surface of the particle, on the interior of the particle, or both. The additional targeting moieties can be immobilized on the surface of the particle, e.g., can be covalently attached to polymer or lipid in the particle. In preferred embodiments, the additional targeting moieties are covalently attached to an amphiphilic polymer or a lipid such that the targeting moieties are oriented on the surface of the particle.

### III. Pharmaceutical Formulations and Vaccines

In some embodiments, conjugates, particles of the present invention may be formulated as vaccines, provided as liquid suspensions or as freeze-dried products. Suitable liquid preparations may include, but are not limited to, isotonic aqueous solutions, suspensions, emulsions, or viscous compositions that are buffered to a selected pH.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit. As used herein, the term “active ingredient” refers to any chemical and biological substance that has a physiological effect in human or in animals, when exposed to it. In the context of the present invention, the active ingredient in the formulations may be any conjugates and particles as discussed herein above.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

The conjugates or particles of the present invention can be formulated using one or more excipients to: (1) increase stability; (2) permit the sustained or delayed release (e.g., from a depot formulation of the monomaleimide); (3) alter the biodistribution (e.g., target the monomaleimide compounds to specific tissues or cell types); (4) alter the release profile of the monomaleimide compounds in vivo. Non-limiting examples of the excipients include any and all solvents, dispersion media, diluents, or other liquid vehicles, dispersion or suspension aids, surface active agents, isotonic agents, thickening or emulsifying agents, and preservatives. Excipients of the present invention may also include, without limitation, lipidoids, liposomes, lipid nanoparticles, polymers, lipoplexes, core-shell nanoparticles, peptides, proteins, hyaluronidase, nanoparticle mimics and combinations thereof. Accordingly, the formulations of the invention may include one or more excipients, each in an amount that together increases the stability of the monomaleimide compounds.

In some embodiments, the conjugates or particles of the present invention are formulated in aqueous formulations such as pH 7.4 phosphate-buffered formulation, or pH 6.2 citrate-buffered formulation; formulations for lyophilization such as pH 6.2 citrate-buffered formulation with 3% mannitol, pH 6.2 citrate-buffered formulation with 4% mannitol/1% sucrose; or a formulation prepared by the process disclosed in U.S. Pat. No. 8,883,737 to Reddy et al. (Endocyte), the contents of which are incorporated herein by reference in their entirety.

In some embodiments, the conjugates or particles of the present invention targets folate receptors and are formulated in liposomes prepared following methods by Leamon et al. in Bioconjugate Chemistry, vol. 14 738-747 (2003), the contents of which are incorporated herein by reference in their entirety. Briefly, folate-targeted liposomes will consist of 40 mole % cholesterol, either 4 mole % or 6 mole % polyethylene glycol (Mr^{˜}2000)-derivatized phosphatidylethanolamine (PEG2000-PE, Nektar, Ala., Huntsville, Ala.), either 0.03 mole % or 0.1 mole % folate-cysteine-PEG3400-PE and the remaining mole % will be composed of egg phosphatidylcholine, as disclosed in U.S. Pat. No. 8,765,096 to Leamon et al. (Endocyte), the contents of which are incorporated herein by reference in their entirety. Lipids in chloroform will be dried to a thin film by rotary evaporation and then rehydrated in PBS containing the drug. Rehydration will be accomplished by vigorous vortexing followed by 10 cycles of freezing and thawing. Liposomes will be extruded 10 times through a 50 nm pore size polycarbonate membrane using a high-pressure extruder. Similarly, liposomes not targeting folate receptors may be prepared identically with the absence of folate-cysteine-PEG3400-PE.

In some embodiments, the conjugates or particles of the present invention are formulated in parenteral dosage forms including but limited to aqueous solutions of the conjugates or particles, in an isotonic saline, 5% glucose or other pharmaceutically acceptable liquid carriers such as liquid alcohols, glycols, esters, and amides, as disclosed in U.S. Pat. No. 7,910,594 to Vlahov et al. (Endocyte), the contents of which are incorporated herein by reference in their entirety. The parenteral dosage form may be in the form of a reconstitutable lyophilizate comprising the dose of the conjugates or particles. Any prolonged release dosage forms known in the art can be utilized such as, for example, the biodegradable carbohydrate matrices described in U.S. Pat. Nos. 4,713,249; 5,266,333; and 5,417,982, the disclosures of which are incorporated herein by reference, or, alternatively, a slow pump (e.g., an osmotic pump) can be used.

In some embodiments, the parenteral formulations are aqueous solutions containing carriers or excipients such as salts, carbohydrates and buffering agents (e.g.,at a pH of from 3 to 9). In some embodiments, the conjugates or particles of the present invention may be formulated as a sterile non-aqueous solution or as a dried form and may be used in conjunction with a suitable vehicle such as sterile, pyrogen-free water. The preparation of parenteral formulations under sterile conditions, for example, by lyophilization under sterile conditions, may readily be accomplished using standard pharmaceutical techniques well-known to those skilled in the art. The solubility of a conjugates or particles used in the preparation of a parenteral formulation may be increased by the use of appropriate formulation techniques, such as the incorporation of solubility-enhancing agents.

In some embodiments, the conjugates or particles of the present invention may be prepared in an aqueous sterile liquid formulation comprising monobasic sodium phosphate monohydrate, dibasic disodium phosphate dihydrate, sodium chloride, potassium chloride and water for injection, as disclosed in US 20140140925 to Leamon et al., the contents of which are incorporated herein by reference in their entirety. For example, the conjugates or particles of the present invention may be formulated in an aqueous liquid of pH 7.4, phosphate buffered formulation for intravenous administration as disclosed in Example 23 of WO2011014821 to Leamon et al. (Endocyte), the contents of which are incorporated herein by reference in their entirety. According to Leamon, the aqueous formulation needs to be stored in the frozen state to ensure its stability.

In some embodiments, the conjugates or particles of the present invention are formulated for intravenous (IV) administration. Any formulation or any formulation prepared according to the process disclosed in US 20140030321 to Ritter et al. (Endocyte), the contents of which are incorporated herein by reference in their entirety, may be used. For example, the conjugates or particles may be formulated in an aqueous sterile liquid formulation of pH 7.4 phosphate buffered composition comprising sodium phosphate, monobasic monohydrate, disodium phosphate, dibasic dehydrate, sodium chloride, and water for injection. As another example, the conjugates or particles may be formulated in pH 6.2 citrated-buffered formulation comprising trisodium citrate, dehydrate, citric acid and water for injection. As another example, the conjugates or particles may be formulated with 3% mannitol in a pH 6.2 citrate-buffered formulation for lyophilization comprising trisodium citrate, dehydrate, citric acid and mannitol. 3% mannitol may be replaced with 4% mannitol and 1% sucrose.

In some embodiments, the particles comprise biocompatible polymers. In some embodiments, the particles comprise about 0.2 to about 35 weight percent of a therapeutic agent; and about 10 to about 99 weight percent of a biocompatible polymer such as a diblock poly(lactic) acid-poly(ethylene)glycol as disclosed in US 20140356444 to Troiano et al. (BIND Therapeutics), the contents of which are incorporated herein by reference in their entirety. Any therapeutically particle composition in U.S. Pat. Nos. 8,663,700, 8,652,528, 8,609,142, 8,293,276 and 8,420,123, the contents of each of which are incorporated herein by reference in their entirety, may also be used.

In some embodiments, the particles comprise a hydrophobic acid. In some embodiments, the particles comprise about 0.05 to about 30 weight percent of a substantially hydrophobic acid; about 0.2 to about 20 weight percent of a basic therapeutic agent having a protonatable nitrogen; wherein the pKa of the basic therapeutic agent is at least about 1.0 pKa units greater than the pKa of the hydrophobic acid; and about 50 to about 99.75 weight percent of a diblock poly(lactic) acid-poly(ethylene)glycol copolymer or a diblock poly(lactic acid-co-glycolic acid)-poly(ethylene)glycol copolymer, wherein the therapeutic nanoparticle comprises about 10 to about 30 weight percent poly(ethylene)glycol as disclosed in WO2014043625 to Figueiredo et al. (BIND Therapeutics), the contents of which are incorporated herein by reference in their entirety. Any therapeutic particle composition in US 20140149158, 20140248358, 20140178475 to Figueiredo et al., the contents of each of which are incorporated herein by reference in their entirety, may also be used.

In some embodiments, the particles comprise a chemotherapeutic agent; a diblock copolymer of poly(ethylene)glycol and polylactic acid; and a ligand conjugate, as disclosed in US 20140235706 to Zale et al. (BIND Therapeutics), the contents of which are incorporated herein by reference in their entirety. Any of the particle compositions in U.S. Pat. Nos. 8,603,501, 8,603,500, 8,603,499, 8,273,363, 8,246,968, 20130172406 to Zale et al., may also be used.

In some embodiments, the particles comprise a targeting moiety. As a non-limiting example, the particles may comprise about 1 to about 20 mole percent PLA-PEG-basement vascular membrane targeting peptide, wherein the targeting peptide comprises PLA having a number average molecular weight of about 15 to about 20 kDa and PEG having a number average molecular weight of about 4 to about 6 kDa; about 10 to about 25 weight percent anti-neointimal hyperplasia (NIH) agent; and about 50 to about 90 weight percent non-targeted poly-lactic acid-PEG, wherein the therapeutic particle is capable of releasing the anti-NIH agent to a basement vascular membrane of a blood vessel for at least about 8 hours when the therapeutic particle is placed in the blood vessel as disclosed in U.S. Pat. No. 8,563,041 to Grayson et al. (BIND Therapeutics), the contents of which are incorporated herein by reference in their entirety.

In some embodiments, the particles comprise about 4 to about 25% by weight of an anti-cancer agent; about 40 to about 99% by weight of poly(D,L-lactic)acid-poly(ethylene)glycol copolymer; and about 0.2 to about 10 mole percent PLA-PEG-ligand; wherein the pharmaceutical aqueous suspension have a glass transition temperature between about 39 and 41° C., as disclosed in U.S. Pat. No. 8,518,963 to Ali et al. (BIND Therapeutics), the contents of which are incorporated herein by reference in their entirety.

In some embodiments, the particles comprise about 0.2 to about 35 weight percent of a therapeutic agent; about 10 to about 99 weight percent of a diblock poly(lactic) acid-poly(ethylene)glycol copolymer or a diblock poly(lactic)-co-poly (glycolic) acid-poly(ethylene)glycol copolymer; and about 0 to about 75 weight percent poly(lactic) acid or poly(lactic) acid-co-poly (glycolic) acid as disclosed in WO2012166923 to Zale et al. (BIND Therapeutics), the contents of which are incorporated herein by reference in their entirety.

In some embodiments, the particles are long circulating and may be formulated in a biocompatible and injectable formulation. For example, the particles may be a sterile, biocompatible and injectable nanoparticle composition comprising a plurality of long circulating nanoparticles having a diameter of about 70 to about 130 nm, each of the plurality of the long circulating nanoparticles comprising about 70 to about 90 weight percent poly(lactic) acid-co-poly(ethylene) glycol, wherein the weight ratio of poly(lactic) acid to poly(ethylene) glycol is about 15 kDa/2 kDa to about 20 kDa/10 kDa, and a therapeutic agent encapsulated in the nanoparticles as disclosed in US 20140093579 to Zale et al. (BIND Therapeutics), the content of which is incorporated herein by reference in its entirety.

In some embodiments, provided is a reconstituted lyophilized pharmaceutical composition suitable for parenteral administration comprising the particles of the present invention. For example, the reconstituted lyophilized pharmaceutical composition may comprise a 10-100 mg/mL concentration of polymeric nanoparticles in an aqueous medium; wherein the polymeric nanoparticles comprise: a poly(lactic) acid-block-poly(ethylene)glycol copolymer or poly(lactic)-co-poly(glycolic) acid-block-poly(ethylene)glycol copolymer, and a taxane agent; 4 to 6 weight percent sucrose or trehalose; and 7 to 12 weight percent hydroxypropyl β-cyclodextrin, as disclosed in U.S. Pat. No. 8,637,083 to Troiano et al. (BIND Therapeutics), the contents of which are incorporated herein by reference in their entirety. Any pharmaceutical composition in U.S. Pat. Nos. 8,603,535, 8,357,401, 20130230568, 20130243863 to Troiano et al. may also be used.

In some embodiments, the conjugates and/or particles of the invention may be delivered with a bacteriophage. For example, a bacteriophage may be conjugated through a labile/non labile linker or directly to at least 1,000 therapeutic drug molecules such that the drug molecules are conjugated to the outer surface of the bacteriophage as disclosed in US 20110286971 to Yacoby et al., the content of which is incorporated herein by reference in its entirety. According to Yacoby et al., the bacteriophage may comprise an exogenous targeting moiety that binds a cell surface molecule on a target cell.

In some embodiments, the conjugates and/or particles of the invention may be delivered with a dendrimer. The conjugates may be encapsulated in a dendrimer, or disposed on the surface of a dendrimer. For example, the conjugates may bind to a scaffold for dendritic encapsulation, wherein the scaffold is covalently or non-covalently attached to a polysaccharide, as disclosed in US 20090036553 to Piccariello et al., the content of which is incorporated herein by reference in its entirety. The scaffold may be any peptide or oligonucleotide scaffold disclosed by Piccariello et al.

In some embodiments, the conjugates and/or particles of the invention may be delivered by a cyclodextrin. In one embodiment, the conjugates may be formulated with a polymer comprising a cyclodextrin moiety and a linker moiety as disclosed in US 20130288986 to Davis et al., the content of which is incorporated herein by reference in its entirety. Davis et al. also teaches that the conjugate may be covalently attached to a polymer through a tether, wherein the tether comprises a self-cyclizing moiety.

In some embodiments, the conjugates and/or particles of the invention may be delivered with an aliphatic polymer. For example, the aliphatic polymer may comprise polyesters with grafted zwitterions, such as polyester-graft-phosphorylcholine polymers prepared by ring-opening polymerization and click chemistry as disclosed in U.S. Pat. No. 8,802,738 to Emrick; the content of which is incorporated herein by reference in its entirety.

### A. Excipients

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEENn®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

### B. Lipidoids

Lipidoids may be used to deliver conjugates of the present invention. Complexes, micelles, liposomes or particles can be prepared containing these lipidoids and therefore, can result in an effective delivery of the conjugates of the present invention, for a variety of therapeutic indications including vaccine adjuvants, following the injection of a lipidoid formulation via localized and/or systemic routes of administration. Lipidoid complexes of conjugates of the present invention can be administered by various means including, but not limited to, intravenous, intramuscular, or subcutaneous routes.

The lipidoid formulations can include particles comprising either 3 or 4 or more components in addition to conjugates of the present invention.

The use of lipidoid formulations for the localized delivery of conjugates to cells (such as, but not limited to, adipose cells and muscle cells) via either subcutaneous or intramuscular delivery, may not require all of the formulation components desired for systemic delivery, and as such may comprise only the lipidoid and the conjugates.

### C. Liposomes, Lipid Nanoparticles and Lipoplexes

The conjugates of the invention can be formulated using one or more liposomes, lipoplexes, or lipid nanoparticles. In one embodiment, pharmaceutical compositions of the conjugates of the invention include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLN) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In one embodiment, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety).

In one embodiment, the conjugates of the invention may be formulated in a lipid vesicle which may have crosslinks between functionalized lipid bilayers.

In one embodiment, the conjugates of the invention may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine and the cationic peptides described in International Pub. No. WO2012013326; herein incorporated by reference in its entirety. In another embodiment, the conjugates of the invention may be formulated in a lipid-polycation complex which may further include a neutral lipid such as, but not limited to, cholesterol or dioleoyl phosphatidylethanolamine (DOPE).

The liposome formulation may be influenced by, but not limited to, the selection of the cationic lipid component, the degree of cationic lipid saturation, the nature of the PEGylation, ratio of all components and biophysical parameters such as size.

In some embodiments, the ratio of PEG in the lipid nanoparticle (LNP) formulations may be increased or decreased and/or the carbon chain length of the PEG lipid may be modified from C14 to C18 to alter the pharmacokinetics and/or biodistribution of the LNP formulations. As a non-limiting example, LNP formulations may contain 1-5% of the lipid molar ratio of PEG-c-DOMG as compared to the cationic lipid, DSPC and cholesterol. In another embodiment the PEG-c-DOMG may be replaced with a PEG lipid such as, but not limited to, PEG-DSG (1,2-Distearoyl-sn-glycerol, methoxypolyethylene glycol) or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In one embodiment, the cationic lipid may be selected from, but not limited to, a cationic lipid described in International Publication Nos. WO2012040184, WO2011153120, WO2011149733, WO2011090965, WO2011043913, WO2011022460, WO2012061259, WO2012054365, WO2012044638, WO2010080724, WO201021865 and WO2008103276, U.S. Pat. Nos. 7,893,302, 7,404,969 and 8,283,333 and US Patent Publication No. US20100036115 and US20120202871; each of which is herein incorporated by reference in their entirety. In another embodiment, the cationic lipid may be selected from, but not limited to, formula A described in International Publication Nos. WO2012040184, WO2011153120, WO2011149733, WO2011090965, WO2011043913, WO2011022460, WO2012061259, WO2012054365 and WO2012044638; each of which is herein incorporated by reference in their entirety. In yet another embodiment, the cationic lipid may be selected from, but not limited to, formula CLI-CLXXIX of International Publication No. WO2008103276, formula CLI-CLXXIX of U.S. Pat. No. 7,893,302, formula CLI-CLXXXXII of U.S. Pat. No. 7,404,969 and formula I-VI of US Patent Publication No. US20100036115; the contents of each of which are herein incorporated by reference in their entirety.

In one embodiment, the cationic lipid may be synthesized by methods known in the art and/or as described in International Publication Nos. WO2012040184, WO2011153120, WO2011149733, WO2011090965, WO2011043913, WO2011022460, WO2012061259, WO2012054365, WO2012044638, WO2010080724 and WO201021865; each of which is herein incorporated by reference in their entirety.

In one embodiment, the LNP formulation may be formulated by the methods described in International Publication Nos. WO2011127255 or WO2008103276, each of which is herein incorporated by reference in their entirety. As a non-limiting example, conjugates described herein may be encapsulated in LNP formulations as described in WO2011127255 and/or WO2008103276; each of which is herein incorporated by reference in their entirety. As another non-limiting example, conjugates described herein may be formulated in a nanoparticle to be delivered by a parenteral route as described in U.S. Pub. No. 20120207845; herein incorporated by reference in its entirety.

The nanoparticle formulations may be a carbohydrate nanoparticle comprising a carbohydrate carrier and a conjugate. As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012109121; herein incorporated by reference in its entirety).

Nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosa tissue within seconds or within a few hours. Large polymeric nanoparticles (200nm-500nm in diameter) which have been coated densely with a low molecular weight polyethylene glycol (PEG) diffused through mucus only 4 to 6-fold lower than the same particles diffusing in water (Lai et al. PNAS 2007 104(5):1482-487; Lai et al. Adv Drug Deliv Rev. 2009 61(2): 158-171; each of which is herein incorporated by reference in their entirety). The transport of nanoparticles may be determined using rates of permeation and/or fluorescent microscopy techniques including, but not limited to, fluorescence recovery after photo bleaching (FRAP) and high resolution multiple particle tracking (MPT). As a non-limiting example, compositions which can penetrate a mucosal barrier may be made as described in U.S. Pat. No. 8,241,670, herein incorporated by reference in its entirety.

Nanoparticle engineered to penetrate mucus may comprise a polymeric material (i.e. a polymeric core) and/or a polymer-vitamin conjugate and/or a tri-block co-polymer. The polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates. The polymeric material may be biodegradable and/or biocompatible. The polymeric material may additionally be irradiated. As a non-limiting example, the polymeric material may be gamma irradiated (See e.g., International App. No. WO201282165, herein incorporated by reference in its entirety). Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), and trimethylene carbonate, polyvinylpyrrolidone. The lipid nanoparticle may be coated or associated with a co-polymer such as, but not limited to, a block co-polymer, and (poly(ethylene glycol))-(poly(propylene oxide))-(poly(ethylene glycol)) triblock copolymer (see e.g., US Publication 20120121718 and US Publication 20100003337 and U.S. Pat. No. 8,263,665; each of which is herein incorporated by reference in their entirety). The co-polymer may be a polymer that is generally regarded as safe (GRAS) and the formation of the lipid nanoparticle may be in such a way that no new chemical entities are created. For example, the lipid nanoparticle may comprise poloxamers coating PLGA nanoparticles without forming new chemical entities which are still able to rapidly penetrate human mucus (Yang et al. Angew. Chem. Int. Ed. 2011 50:2597-2600; herein incorporated by reference in its entirety).

The vitamin of the polymer-vitamin conjugate may be vitamin E. The vitamin portion of the conjugate may be substituted with other suitable components such as, but not limited to, vitamin A, vitamin E, other vitamins, cholesterol, a hydrophobic moiety, or a hydrophobic component of other surfactants (e.g., sterol chains, fatty acids, hydrocarbon chains and alkylene oxide chains).

In one embodiment, the conjugate of the invention is formulated as a lipoplex, such as, without limitation, the ATUPLEX™ system, the DACC system, the DBTC system and other conjugate-lipoplex technology from Silence Therapeutics (London, United Kingdom), STEMFECT™ from STEMGENT® (Cambridge, Mass.), and polyethylenimine (PEI) or protamine-based targeted and non-targeted delivery of therapeutic agents (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293Weide et al. J Immunother. 2009 32:498-507; Weide et al. J Immunother. 2008 31:180-188; Pascolo, Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132; all of which are incorporated herein by reference in its entirety).

In one embodiment such formulations may also be constructed or compositions altered such that they passively or actively are directed to different cell types in vivo, including but not limited to hepatocytes, immune cells (e.g., antigen presenting cells, dendritic cells, T lymphocytes, B lymphocytes, natural killer cells and leukocytes), tumor cells and endothelial cells, (Akinc et al. Mol Ther. 2010 18:1357-1364; Song et al., Nat Biotechnol. 2005 23:709-717; Judge et al., J Chn Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133; all of which are incorporated herein by reference in its entirety). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011, 12:2708-2714; Zhao et al., Expert Opin Drug Deliv. 2008, 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer, J Control Release. 2010, 20:63-68; Peer et al., Proc Natl Acad Sci USA. 2007, 104:4095-4100; Kim et al., Methods Mol Biol. 2011, 721:339-353; Subramanya et al., Mol Ther. 2010, 18:2028-2037; Song et al., Nat Biotechnol. 2005, 23:709-717; Peer et al., Science. 2008, 319:627-630; Peer and Lieberman, Gene Ther. 2011, 18:1127-1133; all of which are incorporated herein by reference in its entirety).

In one embodiment, the conjugates of the invention are formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In a further embodiment, the lipid nanoparticle may be a self-assembly lipid-polymer nanoparticle (see Zhang et al., ACS Nano, 2008, 2 (8), pp 1696-1702; herein incorporated by reference in its entirety).

In one embodiment, the conjugates of the invention can be formulated for controlled release and/or targeted delivery. As used herein, “controlled release” refers to a pharmaceutical composition or compound release profile that conforms to a particular pattern of release to effect a therapeutic outcome. In one embodiment, the conjugates of the invention may be encapsulated into a delivery agent described herein and/or known in the art for controlled release and/or targeted delivery. As used herein, the term “encapsulate” means to enclose, surround or encase. As it relates to the formulation of the conjugates of the invention, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of conjugate of the invention may be enclosed, surrounded or encased within the particle. “Partially encapsulation” means that less than 10, 10, 20, 30, 40 50 or less of the conjugate of the invention may be enclosed, surrounded or encased within the particle. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the particle.

In another embodiment, the conjugates of the invention may be encapsulated into a nanoparticle or a rapidly eliminated nanoparticle and the nanoparticles or a rapidly eliminated nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

In another embodiment, the nanoparticle may be encapsulated into any polymer known in the art which may form a gel when injected into a subject. As a non-limiting example, the nanoparticle may be encapsulated into a polymer matrix which may be biodegradable.

In one embodiment, the conjugate formulation for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In one embodiment, the controlled release and/or targeted delivery formulation may comprise at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In one embodiment, the conjugate of the present invention may be encapsulated in a therapeutic nanoparticle. Therapeutic nanoparticles may be formulated by methods described herein and known in the art such as, but not limited to, International Pub Nos. WO2010005740, WO2010030763, WO2010005721, WO2010005723, WO2012054923, US Pub. Nos. US20110262491, US20100104645, US20100087337, US20100068285, US20110274759, US20100068286 and US20120288541, and U.S. Pat. No. 8,206,747, 8,293,276 8,318,208 and 8,318,211; each of which is herein incorporated by reference in their entirety. In another embodiment, therapeutic polymer nanoparticles may be identified by the methods described in US Pub No. US20120140790, herein incorporated by reference in its entirety.

In one embodiment, the therapeutic nanoparticle may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the conjugate of the present invention (see International Pub No. 2010075072 and US Pub No. US20100216804, US20110217377 and US20120201859, each of which is herein incorporated by reference in their entirety).

In one embodiment, the therapeutic nanoparticles may be formulated to be target specific. As a non-limiting example, the therapeutic nanoparticles may include a corticosteroid (see International Pub. No. WO2011084518 herein incorporated by reference in its entirety). In one embodiment, the therapeutic nanoparticles of the present invention may be formulated to be antiviral immunotherapeutics or vaccine adjuvants. As a non-limiting example, the therapeutic nanoparticles may be formulated in nanoparticles described in International Pub No. WO2008121949, WO2010005726, WO2010005725, WO2011084521 and US Pub No. US20100069426, US20120004293 and US20100104655, each of which is herein incorporated by reference in their entirety.

In one embodiment, the nanoparticles of the present invention may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In one embodiment, the therapeutic nanoparticle comprises a diblock copolymer. In one embodiment, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

As a non-limiting example the therapeutic nanoparticle comprises a PLGA-PEG block copolymer (see US Pub. No. US20120004293 and US Pat No. 8,236,330, each of which is herein incorporated by reference in their entirety). In another non-limiting example, the therapeutic nanoparticle is a stealth nanoparticle comprising a diblock copolymer of PEG and PLA or PEG and PLGA (see U.S. Pat. No. 8,246,968, herein incorporated by reference in its entirety).

In one embodiment, the therapeutic nanoparticle may comprise a multiblock copolymer (See e.g., U.S. Pat. No. 8,263,665 and 8,287,910; each of which is herein incorporated by reference in its entirety).

In one embodiment, the block copolymers described herein may be included in a polyion complex comprising a non-polymeric micelle and the block copolymer. (See e.g., U.S. Pub. No. 20120076836; herein incorporated by reference in its entirety).

In one embodiment, the therapeutic nanoparticle may comprise at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, the therapeutic nanoparticles may comprise at least one cationic polymer described herein and/or known in the art.

In one embodiment, the therapeutic nanoparticles may comprise at least one amine-containing polymer such as, but not limited to polylysine, polyethylene imine, poly(amidoamine) dendrimers, poly(beta-amino esters) (See e.g., U.S. Pat. No. 8,287,849; herein incorporated by reference in its entirety) and combinations thereof.

In one embodiment, the therapeutic nanoparticles may comprise at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In another embodiment, the therapeutic nanoparticle may include a conjugation of at least one targeting ligand. The targeting ligand may be any ligand known in the art such as, but not limited to, a monoclonal antibody. (Kirpotin et al, Cancer Res. 2006 66:6732-6740; herein incorporated by reference in its entirety).

In one embodiment, the therapeutic nanoparticle may be formulated in an aqueous solution which may be used to target cancer (see International Pub No. WO2011084513 and US Pub No. US20110294717, each of which is herein incorporated by reference in their entirety).

In one embodiment, the conjugates of the invention may be encapsulated in, linked to and/or associated with synthetic nanocarriers. Synthetic nanocarriers include, but are not limited to, those described in International Pub. Nos. WO2010005740, WO2010030763, WO201213501, WO2012149252, WO2012149255, WO2012149259, WO2012149265, WO2012149268, WO2012149282, WO2012149301, WO2012149393, WO2012149405, WO2012149411 and WO2012149454 and US Pub. Nos. US20110262491, US20100104645, US20100087337 and US20120244222, each of which is herein incorporated by reference in their entirety. The synthetic nanocarriers may be formulated using methods known in the art and/or described herein. As a non-limiting example, the synthetic nanocarriers may be formulated by the methods described in International Pub Nos. WO2010005740, WO2010030763 and WO201213501 and US Pub. Nos. US20110262491, US20100104645, US20100087337 and US20120244222, each of which is herein incorporated by reference in their entirety. In another embodiment, the synthetic nanocarrier formulations may be lyophilized by methods described in International Pub. No. WO2011072218 and US Pat No. 8,211,473; each of which is herein incorporated by reference in their entirety.

In one embodiment, the synthetic nanocarriers may contain reactive groups to release the conjugates described herein (see International Pub. No. WO20120952552 and US Pub No. US20120171229, each of which is herein incorporated by reference in their entirety).

In one embodiment, the synthetic nanocarriers may be formulated for targeted release. In one embodiment, the synthetic nanocarrier is formulated to release the conjugates at a specified pH and/or after a desired time interval. As a non-limiting example, the synthetic nanoparticle may be formulated to release the conjugates after 24 hours and/or at a pH of 4.5 (see International Pub. Nos. WO2010138193 and WO2010138194 and US Pub Nos. US20110020388 and US20110027217, each of which is herein incorporated by reference in their entirety).

In one embodiment, the synthetic nanocarriers may be formulated for controlled and/or sustained release of conjugates described herein. As a non-limiting example, the synthetic nanocarriers for sustained release may be formulated by methods known in the art, described herein and/or as described in International Pub No. WO2010138192 and US Pub No. 20100303850, each of which is herein incorporated by reference in their entirety.

In one embodiment, the nanoparticle may be optimized for oral administration. The nanoparticle may comprise at least one cationic biopolymer such as, but not limited to, chitosan or a derivative thereof. As a non-limiting example, the nanoparticle may be formulated by the methods described in U.S. Pub. No. 20120282343; herein incorporated by reference in its entirety.

### D. Polymers, Biodegradable Nanoparticles, and Core-Shell Nanoparticles

The conjugates of the invention can be formulated using natural and/or synthetic polymers. Non-limiting examples of polymers which may be used for delivery include, but are not limited to, DYNAMIC POLYCONJUGATE® (Arrowhead Research Corp., Pasadena, Calif.) formulations from MIRUS® Bio (Madison, Wis.) and Roche Madison (Madison, Wis.), PHASERX™ polymer formulations such as, without limitation, SMARTT POLYMER TECHNOLOGY™ (Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly(lactic-co-glycolic acid) (PLGA) polymers, RONDEL™ (RNAi/Oligonucleotide Nanoparticle Delivery) polymers (Arrowhead Research Corporation, Pasadena, Calif.) and pH responsive co-block polymers such as, but not limited to, PHASERX™ (Seattle, Wash.).

A non-limiting example of chitosan formulation includes a core of positively charged chitosan and an outer portion of negatively charged substrate (U.S. Pub. No. 20120258176; herein incorporated by reference in its entirety). Chitosan includes, but is not limited to N-trimethyl chitosan, mono-N-carboxymethyl chitosan (MCC), N-palmitoyl chitosan (NPCS), EDTA-chitosan, low molecular weight chitosan, chitosan derivatives, or combinations thereof.

In one embodiment, the polymers used in the present invention have undergone processing to reduce and/or inhibit the attachment of unwanted substances such as, but not limited to, bacteria, to the surface of the polymer. The polymer may be processed by methods known and/or described in the art and/or described in International Pub. No. WO2012150467, herein incorporated by reference in its entirety.

A non-limiting example of PLGA formulations include, but are not limited to, PLGA injectable depots (e.g., ELIGARD® which is formed by dissolving PLGA in 66% N-methyl-2-pyrrolidone (NMP) and the remainder being aqueous solvent and leuprolide. Once injected, the PLGA and leuprolide peptide precipitates into the subcutaneous space).

In one embodiment, the pharmaceutical compositions may be sustained release formulations. In a further embodiment, the sustained release formulations may be for subcutaneous delivery. Sustained release formulations may include, but are not limited to, PLGA microspheres, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

As a non-limiting example modified mRNA may be formulated in PLGA microspheres by preparing the PLGA microspheres with tunable release rates (e.g., days and weeks) and encapsulating the conjugate in the PLGA microspheres while maintaining the integrity of the conjugate during the encapsulation process. EVAc are non-biodegradable, biocompatible polymers which are used extensively in pre-clinical sustained release implant applications (e.g., extended release products Ocusert a pilocarpine ophthalmic insert for glaucoma or progestasert a sustained release progesterone intrauterine device; transdermal delivery systems Testoderm, Duragesic and Selegiline; catheters). Poloxamer F-407 NF is a hydrophilic, non-ionic surfactant triblock copolymer of polyoxyethylene-polyoxypropylene-polyoxyethylene having a low viscosity at temperatures less than 5° C. and forms a solid gel at temperatures greater than 15° C. PEG-based surgical sealants comprise two synthetic PEG components mixed in a delivery device which can be prepared in one minute, seals in 3 minutes and is reabsorbed within 30 days. GELSI1E® and natural polymers are capable of in-situ gelation at the site of administration. They have been shown to interact with protein and peptide therapeutic candidates through ionic interaction to provide a stabilizing effect.

Polymer formulations can also be selectively targeted through expression of different ligands as exemplified by, but not limited by, folate, transferrin, and N-acetylgalactosamine (GalNAc) (Benoit et al., Biomacromolecules. 2011 12:2708-2714; Rozema et al., Proc Natl Acad Sci USA. 2007 104:12982-12887; Davis, Mol Pharm. 2009, 6:659-668; Davis, Nature, 2010, 464:1067-1070; each of which is herein incorporated by reference in its entirety).

The conjugates of the invention may be formulated with or in a polymeric compound. The polymer may include at least one polymer such as, but not limited to, polyethenes, polyethylene glycol (PEG), poly(l-lysine)(PLL), PEG grafted to PLL, cationic lipopolymer, biodegradable cationic lipopolymer, polyethylenimine (PEI), cross-linked branched poly(alkylene imines), a polyamine derivative, a modified poloxamer, a biodegradable polymer, elastic biodegradable polymer, biodegradable block copolymer, biodegradable random copolymer, biodegradable polyester copolymer, biodegradable polyester block copolymer, biodegradable polyester block random copolymer, multiblock copolymers, linear biodegradable copolymer, poly[α-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers, dextran polymers, dextran polymer derivatives or combinations thereof.

As a non-limiting example, the conjugate of the invention may be formulated with the polymeric compound of PEG grafted with PLL as described in U.S. Pat. No. 6,177,274; herein incorporated by reference in its entirety. In another example, the conjugate may be suspended in a solution or medium with a cationic polymer, in a dry pharmaceutical composition or in a solution that is capable of being dried as described in U.S. Pub. Nos. 20090042829 and 20090042825; each of which are herein incorporated by reference in their entireties.

As another non-limiting example the conjugate of the invention may be formulated with a PLGA-PEG block copolymer (see US Pub. No. US20120004293 and U.S. Pat. No. 8,236,330, each of which are herein incorporated by reference in their entireties) or PLGA-PEG-PLGA block copolymers (See U.S. Pat. No. 6,004,573, herein incorporated by reference in its entirety). As a non-limiting example, the conjugate of the invention may be formulated with a diblock copolymer of PEG and PLA or PEG and PLGA (see U.S. Pat. No 8,246,968, herein incorporated by reference in its entirety).

A polyamine derivative may be used to deliver conjugates of the invention or to treat and/or prevent a disease or to be included in an implantable or injectable device (U.S. Pub. No. 20100260817 herein incorporated by reference in its entirety). As a non-limiting example, a pharmaceutical composition may include the conjugates of the invention and the polyamine derivative described in U.S. Pub. No. 20100260817 (the contents of which are incorporated herein by reference in its entirety). As a non-limiting example the conjugates of the invention may be delivered using a polyamide polymer such as, but not limited to, a polymer comprising a 1,3-dipolar addition polymer prepared by combining a carbohydrate diazide monomer with a dilkyne unite comprising oligoamines (U.S. Pat. No. 8,236,280; herein incorporated by reference in its entirety).

The conjugate of the invention may be formulated with at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, the conjugates of the invention may be formulated with at least one polymer and/or derivatives thereof described in International Publication Nos. WO2011115862, WO2012082574 and WO2012068187 and U.S. Pub. No. 20120283427, each of which are herein incorporated by reference in their entireties. In another embodiment, the conjugates of the invention may be formulated with a polymer of formula Z as described in WO2011115862, herein incorporated by reference in its entirety. In yet another embodiment, the conjugates of the invention may be formulated with a polymer of formula Z, Z′ or Z″ as described in International Pub. Nos. WO2012082574 or WO2012068187, each of which are herein incorporated by reference in their entireties. The polymers formulated with the conjugates of the present invention may be synthesized by the methods described in International Pub. Nos. WO2012082574 or WO2012068187, each of which are herein incorporated by reference in their entireties.

Formulations of conjugates of the invention may include at least one amine-containing polymer such as, but not limited to polylysine, polyethylene imine, poly(amidoamine) dendrimers or combinations thereof.

For example, the conjugate of the invention may be formulated in a pharmaceutical compound including a poly(alkylene imine), a biodegradable cationic lipopolymer, a biodegradable block copolymer, a biodegradable polymer, or a biodegradable random copolymer, a biodegradable polyester block copolymer, a biodegradable polyester polymer, a biodegradable polyester random copolymer, a linear biodegradable copolymer, PAGA, a biodegradable cross-linked cationic multi-block copolymer or combinations thereof. The biodegradable cationic lipopolymer may be made by methods known in the art and/or described in U.S. Pat. No. 6,696,038, U.S. App. Nos. 20030073619 and 20040142474 each of which is herein incorporated by reference in their entireties. The poly(alkylene imine) may be made using methods known in the art and/or as described in U.S. Pub. No. 20100004315, herein incorporated by reference in its entirety. The biodegradable polymer, biodegradable block copolymer, the biodegradable random copolymer, biodegradable polyester block copolymer, biodegradable polyester polymer, or biodegradable polyester random copolymer may be made using methods known in the art and/or as described in U.S. Pat. Nos. 6,517,869 and 6,267,987, the contents of which are each incorporated herein by reference in their entirety. The linear biodegradable copolymer may be made using methods known in the art and/or as described in U.S. Pat. No. 6,652,886. The PAGA polymer may be made using methods known in the art and/or as described in U.S. Pat. No. 6,217,912 herein incorporated by reference in its entirety. The PAGA polymer may be copolymerized to form a copolymer or block copolymer with polymers such as but not limited to, poly-L-lysine, polyarginine, polyornithine, histones, avidin, protamines, polylactides and poly(lactide-co-glycolides). The biodegradable cross-linked cationic multi-block copolymers may be made my methods known in the art and/or as described in U.S. Pat. No. 8,057,821 or U.S. Pub. No. 2012009145 each of which are herein incorporated by reference in their entireties. For example, the multi-block copolymers may be synthesized using linear polyethylenimine (LPEI) blocks which have distinct patterns as compared to branched polyethyleneimines. Further, the composition or pharmaceutical composition may be made by the methods known in the art, described herein, or as described in U.S. Pub. No. 20100004315 or U.S. Pat. Nos. 6,267,987 and 6,217,912 each of which are herein incorporated by reference in their entireties.

The conjugates of the invention may be formulated with at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

The conjugate of the invention may be formulated with at least one cross linkable polyester. Cross linkable polyesters include those known in the art and described in US Pub. No. 20120269761, herein incorporated by reference in its entirety.

In one embodiment, the polymers described herein may be conjugated to a lipid-terminating PEG. As a non-limiting example, PLGA may be conjugated to a lipid-terminating PEG forming PLGA-DSPE-PEG. As another non-limiting example, PEG conjugates for use with the present invention are described in International Publication No. WO2008103276, herein incorporated by reference in its entirety. The polymers may be conjugated using a ligand conjugate such as, but not limited to, the conjugates described in U.S. Pat. No. 8,273,363, herein incorporated by reference in its entirety.

In one embodiment, the conjugates of the invention may be conjugated with another compound. Non-limiting examples of conjugates are described in U.S. Pat. Nos. 7,964,578 and 7,833,992, each of which are herein incorporated by reference in their entireties. In another embodiment, the conjugates of the invention may be conjugated with conjugates of formula 1-122 as described in U.S. Pat. Nos. 7,964,578 and 7,833,992, each of which are herein incorporated by reference in their entireties. The modified RNA described herein may be conjugated with a metal such as, but not limited to, gold. (See e.g., Giljohann et al. Journ. Amer. Chem. Soc. 2009 131(6): 2072-2073; herein incorporated by reference in its entirety). In another embodiment, the conjugates of the invention may be conjugated and/or encapsulated in gold-nanoparticles. (Interantional Pub. No. WO201216269 and U.S. Pub. No. 20120302940; each of which is herein incorporated by reference in its entirety).

In one embodiment, the polymer formulation of the present invention may be stabilized by contacting the polymer formulation, which may include a cationic carrier, with a cationic lipopolymer which may be covalently linked to cholesterol and polyethylene glycol groups. The polymer formulation may be contacted with a cationic lipopolymer using the methods described in U.S. Pub. No. 20090042829 herein incorporated by reference in its entirety. The cationic carrier may include, but is not limited to, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B-[N—(N′,N′-Dimethylaminoethane)-carbamoyl]Cholesterol Hydrochloride (DC-Cholesterol HCl) diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride DODAC) and combinations thereof.

The conjugates of the invention may be formulated in a polyplex of one or more polymers (U.S. Pub. No. 20120237565 and 20120270927; each of which is herein incorporated by reference in its entirety). In one embodiment, the polyplex comprises two or more cationic polymers. The catioinic polymer may comprise a poly(ethylene imine) (PEI) such as linear PEI.

The conjugates of the invention can also be formulated as a nanoparticle using a combination of polymers, lipids, and/or other biodegradable agents, such as, but not limited to, calcium phosphate. Components may be combined in a core-shell, hybrid, and/or layer-by-layer architecture, to allow for fine-tuning of the nanoparticle so that delivery of the conjugates of the invention may be enhanced (Wang et al., Nat Mater. 2006, 5:791-796; Fuller et al., Biomaterials. 2008, 29:1526-1532; DeKoker et al., Adv Drug Deliv Rev. 2011, 63:748-761; Endres et al., Biomaterials. 2011, 32:7721-7731; Su et al., Mol Pharm. 2011, Jun. 6; 8(3):774-87; each of which is herein incorporated by reference in its entirety). As a non-limiting example, the nanoparticle may comprise a plurality of polymers such as, but not limited to hydrophilic-hydrophobic polymers (e.g., PEG-PLGA), hydrophobic polymers (e.g., PEG) and/or hydrophilic polymers (International Pub. No. WO20120225129; herein incorporated by reference in its entirety).

Biodegradable calcium phosphate nanoparticles in combination with lipids and/or polymers have been shown to deliver therapeutic agents in vivo. In one embodiment, a lipid coated calcium phosphate nanoparticle, which may also contain a targeting ligand such as anisamide, may be used to deliver the conjugate of the present invention. For example, to effectively deliver a therapeutic agent in a mouse metastatic lung model a lipid coated calcium phosphate nanoparticle was used (Li et al., J Contr Rel. 2010, 142: 416-421; Li et al., J Contr Rel. 2012, 158:108-114; Yang et al., Mol Ther. 2012, 20:609-615; herein incorporated by refereince in its entirety). This delivery system combines both a targeted nanoparticle and a component to enhance the endosomal escape, calcium phosphate, in order to improve delivery of the therapeutic agent.

In one embodiment, a PEG-charge-conversional polymer (Pitella et al., Biomaterials. 2011, 32:3106-3114) may be used to form a nanoparticle to deliver the conjugate of the present invention. The PEG-charge-conversional polymer may improve upon the PEG-polyanion block copolymers by being cleaved into a polycation at acidic pH, thus enhancing endosomal escape.

The use of core-shell nanoparticles has additionally focused on a high-throughput approach to synthesize cationic cross-linked nanogel cores and various shells (Siegwart et al., Proc Natl Acad Sci USA. 2011, 108:12996-13001). The complexation, delivery, and internalization of the polymeric nanoparticles can be precisely controlled by altering the chemical composition in both the core and shell components of the nanoparticle. For example, the core-shell nanoparticles may efficiently deliver a therapeutic agent to mouse hepatocytes after they covalently attach cholesterol to the nanoparticle.

The use of core-shell nanoparticles has additionally focused on a high-throughput approach to synthesize cationic cross-linked nanogel cores and various shells (Siegwart et al., Proc Natl Acad Sci USA. 2011, 108:12996-13001). The complexation, delivery, and internalization of the polymeric nanoparticles can be precisely controlled by altering the chemical composition in both the core and shell components of the nanoparticle. For example, the core-shell nanoparticles may efficiently deliver a therapeutic agent to mouse hepatocytes after they covalently attach cholesterol to the nanoparticle.

In one embodiment, the lipid nanoparticles may comprise a core of the conjugates disclosed herein and a polymer shell. The polymer shell may be any of the polymers described herein and are known in the art. In an additional embodiment, the polymer shell may be used to protect the modified nucleic acids in the core.

Core-shell nanoparticles for use with the conjugates of the present invention are described and may be formed by the methods described in U.S. Pat. No. 8,313,777 herein incorporated by reference in its entirety.

### E. Inorganic Nanoparticles

Inorganic nanoparticles exhibit a combination of physical, chemical, optical and electronic properties and provide a highly multifunctional platform to image and diagnose diseases, to selectively deliver therapeutic agens, and to sensitive cells and tissues to treatment regiments. Not wishing to be bound to any theory, enhanced permeability and retention (EPR) effect provides a basis for the selective accumulation of many high-molecular-weight drugs. Circulating inorganic nanoparticles preferentially accumulate at tumor sites and in inflamed tissues (Yuan et al., Cancer Res., vol. 55(17):3752-6, 1995, the contents of which are incorporated herein by reference in their entirety) and remain lodged due to their low diffusivity (Pluen et al., PNAS, vol. 98(8):4628-4633, 2001, the contents of which are incorporated herein by reference in their entirety). The size of the inorganic nanoparticles may be 10 nm-500 nm, 10 nm-100 nm or 100 nm-500 nm. The inorganic nanoparticles may comprise metal (gold, iron, silver, copper, nickel, etc.), oxides (ZnO, TiO₂, Al₂O₃, SiO₂, iron oxide, copper oxide, nickel oxide, etc.), or semiconductor (CdS, CdSe, etc.). The inorganic nanoparticles may also be perfluorocarbon or FeCo.

Inorganic nanoparticles have high surface area per unit volume. Therefore, they may be loaded with therapeutic drugs and imaging agents at high densitives. A variety of methods may be used to load therapeutic drugs into/onto the inorganic nanoparticles, including but not limited to, colvalent bonds, electrostatic interactions, entrapment, and encapsulation. In addition to therapeutic agent drug loads, the inorganic nanoparticles may be funcationalized with targeting moieties, such as tumor-targeting ligands, on the surface. Formulating therapeutic agents with inorganic nanoparticles allows imaging, detection and monitoring of the therapeutic agents.

In one embodiment, the conjugate of the invention is hydrophobic and may be form a kinetically stable complex with gold nanoparticles funcationalized with water-soluble zwitterionic ligands disclosed by Kim et al. (Kim et al., JACS, vol. 131(4):1360-1361, 2009, the contents of which are incorporated herein by reference in their entirety). Kim et al. demonstrated that hydrophobic drugs carried by the gold nanoparticles are efficiently released into cells with little or no cellular uptake of the gold nanoparticles.

In one embodiment, the conjugates of the invention may be formulated with gold nanoshells. As a non-limiting example, the conjugates may be delivered with a temperature sensitive system comprising polymers and gold nanoshells and may be released photothermally. Sershen et al. designed a delivery vehicle comprising hydrogel and gold nanoshells, wherein the hydrogels are made of copolymers of N-isopropylacrylamide (NIPAAm) and acrylamide (AAm) and the gold nanoshells are made of gold and gold sulfide (Sershen et al., J Biomed Mater, vol. 51:293-8, 2000, the contents of which are incorporated herein by reference in their entirety). Irradiation at 1064 nm was absorbed by the nanoshells and converted to heat, which led to the collapse of the hydrogen and release of the drug. The conjugate of the invention may also be encapsulated inside hollow gold nanoshells.

In some embodiments, the conjugates of the invention may be attached to gold nanoparticles via covalent bonds. Covalent attachment to gold nanoparticles may be achieved through a linker, such as a free thiol, amine or carboxylate functional group. In some embodiments, the linkers are located on the surface of the gold nanoparticles. In some embodiments, the conjugates of the invention may be modified to comprise the linkers. The linkers may comprise a PEG or oligoethylene glycol moiety with varying length to increase the particles' stability in biological environment and to control the density of the drug loads. PEG or oligoethylene glycol moieties also minimize nonspecific adsorption of undesired biomolecules. PEG or oligoethylene gycol moieties may be branched or linear. Tong et al. disclosed that branched PEG moieties on the surface of gold nanoparticles increase circulatory half-life of the gold nanoparticles and reduced serum protein binding (Tong et al., Langmuir, vol. 25(21):12454-9, 2009, the contents of which are incorporated herein by reference in their entirety).

In one embodiment, the conjugate of the invention may comprise PEG-thiol groups and may attach to gold nanoparticles via the thiol group. The synthesis of thiol-PEGylated conjugates and the attachment to gold nanoparticles may follow the method disclosed by El-Sayed et al. (El-Sayed et al., Bioconjug. Chem., vol. 20(12):2247-2253, 2010, the contents of which are incorporated herein by reference in their entirety).

In another embodiment, the conjugate of the invention may be tethered to an amine-functionalized gold nanoparticles. Lippard et al. disclosed that Pt(IV) prodrugs may be delivered with amine-functionalized polyvalent oligonucleotide gold nanoparticles and are only activated into their active Pt(II) forms after crossing the cell membrane and undergoing intracellular reduction (Lippard et al., JACS, vol. 131(41):14652-14653, 2009, the contents of which are incorporated herein by reference in their entirety). The cytotoxic effects for the Pt(IV)-gold nanoparticle complex are higher than the free Pt(IV) drugs and free cisplatin.

In some embodiments, conjugates of the invention are formulated with magnetic nanoparticle such as iron, cobalt, nickel and oxides thereof, or iron hydroxide nanoparticles. Localized magnetic field gradients may be used to attract magnetic nanoparticles to a chosen site, to hold them until the therapy is complete, and then to remove them. Magnetic nanoparticles may also be heated by magnetic fields. Alexiou et al. prepared an injection of magnetic particle, Ferro fluids (FFs), bound to anticancer agents and then concentrated the particles in the desired tumor area by an external magnetic field (Alexiou et al., Cancer Res. vol. 60(23):6641-6648, 2000, the contents of which are incorporated herein by reference in their entirety). The desorption of the anticancer agent took place within 60 min to make sure that the drug can act freely once localized to the tumor by the magnetic field.

In some embodiments, the conjugates of the invention are loaded onto iron oxide nanoparticles. In some embodiments, the conjugates of the invention are formulated with super paramagnetic nanoparticles based on a core consisting of iron oxides (SPION). SPION are coated with inorganic materials (silica, gold, etc.) or organic materials (phospholipids, fatty acids, polysaccharides, peptides or other surfactants and polymers) and can be further functionalized with drugs, proteins or plasmids.

In one embodiment, water-dispersible oleic acid (OA)-poloxamer-coated iron oxide magnetic nanoparticles disclosed by Jain et al. (Jain, Mol. Pharm., vol. 2(3):194-205, 2005, the contents of which are incorporated herein by reference in their entirety) may be used to deliver the conjugates of the invention. Therapeutic drugs partition into the OA shell surrounding the iron oxide nanoparticles and the poloxamer copolymers (i.e., Pluronics) confers aqueous dispersity to the formulation. According to Jain et al., neither the formulation components nor the drug loading affected the magnetic properties of the core iron oxide nanoparticles. Sustained release of the therapeutic drugs was achieved.

In one embodiment, the conjugates of the invention are bonded to magnetic nanoparticles with a linker. The linker may be a linker capable of undergoing an intramolecular cyclization to release the conjugates of the invention. Any linker and nanoparticles disclosed in WO2014124329 to Knipp et al., the contents of which are incorporated herein by reference in their entirety, may be used. The cyclization may be induced by heating the magnetic nanoparticle or by application of an alternating electromagnetic field to the magnetic nanoparticle.

In one embodiment, the conjugates of the invention may be delivered with a drug delivery system disclosed in U.S. Pat. No. 7,329,638 to Yang et al., the contents of which are incorporated herein by reference in their entirety. The drug delivery system comprises a magnetic nanoparticle associated with a positively charged cationic molecule, at least one therapeutic agent and a molecular recognition element.

In one embodiment, nanoparticles having a phosphate moiety are used to deliver the conjugates of the invention. The phosphate-containing nanoparticle disclosed in U.S. Pat. No. 8,828,975 to Hwu et al., the contents of which are incorporated herein by reference in their entirety, may be used. The nanoparticles may comprise gold, iron oxide, titanium dioxide, zinc oxide, tin dioxide, copper, aluminum, cadmium selenide, silicon dioxide or diamond. The nanoparticles may contain a PEG moiety on the surface.

### E. Peptides and Proteins

The conjugate of the invention can be formulated with peptides and/or proteins in order to increase penetration of cells by the conjugates of the invention. In one embodiment, peptides such as, but not limited to, cell penetrating peptides and proteins and peptides that enable intracellular delivery may be used to deliver pharmaceutical formulations. A non-limiting example of a cell penetrating peptide which may be used with the pharmaceutical formulations of the present invention include a cell-penetrating peptide sequence attached to polycations that facilitates delivery to the intracellular space, e.g., HIV-derived TAT peptide, penetratins, transportans, or hCT derived cell-penetrating peptides (see, e.g., Caron et al., Mol. Ther. 3(3):310-8 (2001); Langel, Cell-Penetrating Peptides: Processes and Applications (CRC Press, Boca Raton Fla., 2002); El-Andaloussi et al., Curr. Pharm. Des. 2003, 11(28):3597-611; and Deshayes et al., Cell. Mol. Life Sci. 2005, 62(16):1839-49, all of which are incorporated herein by reference). The compositions can also be formulated to include a cell penetrating agent, e.g., liposomes, which enhance delivery of the compositions to the intracellular space. The conjugates of the invention may be complexed to peptides and/or proteins such as, but not limited to, peptides and/or proteins from Aileron Therapeutics (Cambridge, Mass.) and Permeon Biologics (Cambridge, Mass.) in order to enable intracellular delivery (Cronican et al., ACS Chem. Biol. 2010, 5:747-752; McNaughton et al., Proc. Natl. Acad. Sci. USA 2009, 106:6111-6116; Sawyer, Chem Biol Drug Des. 2009, 73:3-6; Verdine and Hilinski, Methods Enzymol. 2012, 503:3-33; all of which are herein incorporated by reference in its entirety). In one embodiment, the cell-penetrating polypeptide may comprise a first domain and a second domain. The first domain may comprise a supercharged polypeptide. The second domain may comprise a protein-binding partner. As used herein, “protein-binding partner” includes, but are not limited to, antibodies and functional fragments thereof, scaffold proteins, or peptides. The cell-penetrating polypeptide may further comprise an intracellular binding partner for the protein-binding partner. The cell-penetrating polypeptide may be capable of being secreted from a cell where conjugates of the invention may be introduced.

### IV. Administration, Dose and Dosage Form

Administration: Compositions and formulations containing an effective amount of conjugates or particles of the present invention may be administered to a subject in need thereof by any route which results in a therapeutically effective outcome in said subject. These include, but are not limited to enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions may be administered in a way which allows them cross the blood-brain barrier, vascular barrier, or other epithelial barrier.

In some embodiments, particles, nanoparticles and/or polymerica nanoparticles are administered to bone marrow. In some embodiments, particles, nanoparticles and/or polymerica nanoparticles are administered to areas having a lot of dendritic cells, such as subcutaneous space.

Dose and Dosage forms: Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, compositions in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

As used herein, a “split dose” is the division of single unit dose or total daily dose into two or more doses, e.g., two or more administrations of the single unit dose. As used herein, a “single unit dose” is a dose of any therapeutic administed in one dose/at one time/single route/single point of contact, i.e., single administration event. As used herein, a “total daily dose” is an amount given or prescribed in 24 hr. period. It may be administered as a single unit dose.

A pharmaceutical composition described herein can be formulated into a dosage form described herein, such as a topical, intranasal, intratracheal, or injectable (e.g., intravenous, intraocular, intravitreal, intramuscular, intracardiac, intraperitoneal, and subcutaneous).

In some embodiments, the dosage forms may be liquid dosage forms. Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art including, but not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, compositions may be mixed with solubilizing agents such as CREMOPHOR®, alcohols, oils, modified oils, glycols, polysorbates, cyclodextrins, polymers, and/or combinations thereof.

In certain embodiments, the dosages forms may be injectable. Injectable preparations, for example, sterile injectable aqueous or oleaginous suspensions may be formulated according to the known art and may include suitable dispersing agents, wetting agents, and/or suspending agents. Sterile injectable preparations may be sterile injectable solutions, suspensions, and/or emulsions in nontoxic parenterally acceptable diluents and/or solvents, for example, a solution in 1,3-butanediol. Among the acceptable vehicles and solvents that may be employed include, but are not limited to, water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables. Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it may be desirable to slow the absorption of the active ingredient from subcutaneous or intramuscular injection. This may be accomplished by the use of a liquid suspension of crystalline or amorphous material with poor water solubility. The rate of absorption of the compounds then depends upon its rate of dissolution which, in turn, may depend upon crystal size and crystalline form. Injectable depot forms are made by forming microencapsule matrices of the conjugates in biodegradable polymers such as polylactide-polyglycolide. Depending upon the ratio of conjugates to polymer and the nature of the particular polymer employed, the rate of active agents in the conjugates can be controlled. Examples of other biodegradable polymers include, but are not limited to, poly(orthoesters) and poly(anhydrides). Depot injectable formulations may be prepared by entrapping the conjugates in liposomes or microemulsions which are compatible with body tissues.

In some embodiments, solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

### Immune Modulation

Modulation of the tumor immunosuppressive microenvironment have been proven to be an effective approach for cancer immunotherapy. Antibody based blockade of the T cell co-inhibitory receptor cytotoxic T lymphocyte antigen-4 (CTLA-4) has become the first FDA approved immune checkpoint blockade for melanoma treatment. In addition to CTLA-4, tumor cells and tumor infiltrating immune cells express a diverse array of additional coinhibitory and co-stimulatory signal molecules, which can be targeted to boost tumor immunity. Many studies have indicated that blocking one or more these coinhibitory signal molecules, alone or in conjunction with other immunotherapeutic agents that aim to increase antigen presentation, dendritic cell activation and effector T cell activation, can enhance cancer specific immune responses. For example, the combined inhibition of PD-1 and LAG-3 can generate a synergistically effect (Okazaki et al., PD-1 and LAG-3 inhibitory co-receptors act synergistically to prevent autoimmunity in mice. J Exp. Med. 2011, 208(2): 395-407). Immunomodulation therapies can target TB lymphocytes, macrophages, dendritic cells, natural killer cells (NK Cell), or subsets of these cells such as cytotoxic T lymphocytes (CTL) or Natural Killer T (NKT) cells. Because of interacting immune cascades, an effect on one set of immune cells will often be amplified by spreading to other cells.

In some embodiments, the conjugate of the present invention comprising at least one antagonist agent against the co-inhibitory signal molecules as payloads may be used for immunotherapy. In other embodiments, two or more conjugates each of which comprising an antagonist agent against a co-inhibitory signal molecule may be formulated in one nanoparticle of the present invention for immunotherapy. The antagonist agent may be an antagonistic antibody and a functional antibody fragment/variant thereof, a fusion polypeptide, a soluble peptide of the coinhibitory signal molecule, and/or a small molecule inhibitor that specifically bind to a co-inhibitory signal molecule. The coinhibitory signal molecule is selected from the group consisting of CTLA-4, PD-1, PD-L1, PD-L2, TIM-3, LAG-3, BTLA, CD160, C200R, TIGIT, KLRG-1, KIR, 2B4/CD244, VISTA and Ara2R.

In some embodiments, conjugates, nanoparticles and formulations of the present invention may comprise two or three agents against two or three different coinhibitory signal molecules selected from CTLA-4, PD-1, PD-L1, PD-L2, TIM-3, LAG-3, BTLA, CD160, C200R, TIGIT, KLRG-1, KIR, 2B4/CD244, VISTA and Ara2R, for dual or triple checkpoint inhibition.

In some embodiments, the conjugate of the present invention may comprise at least one antagonist agent specific to a coinhibitory signal molecule and at least one agonist agent specific to a costimulatory signal molecule as payloads for modulating a cancer specific immune response. The antagonist agent of the conjugate can inhibit an immunosuppressive regulatory signal and the agonist agent in the same conjugate can activate an immuno-potentiating signal; the combined effect tips the balance of the immunoregulation towards a positive immune response. In other embodiments, a conjugate comprising an antagonist agent specific to a coinhibitory molecule and a conjugate comprising an agonist agent specific to a costimulatory signal may be formulated into a single nanoparticle of the present invention to generate the same effect. The costimulatory signal molecule may include, but are not limited to CD28, CD80 (B7.1), CD86 (B7.2); 4-1BB (CD137) and its ligand 4-1BBL (CD137L), CD27, CD70, OX40 and its ligand OX40L, GITR and its ligand GITRL, CD40 and CD40 ligand, CD30 and CD30 ligand, CD226, LIGHT, LTβR, LTαβ, ICOS (CD278), ICOSL (B7-H2), NKG2D, an active receptor on NK cells. In some examples, the agonist agent may be an agonistic antibody that specifically binds to one of the co-stimulatory signal molecule, or a functional fragment /variant thereof.

In some embodiments, compositions of the present invention may be used to inhibit the coinhibitory signals that regulate T cell activation. The conjugates will comprise at least one, preferably two antagonist agents specific to CTLA-4, PD-1, PD-L1, PD-L2, TIM-3, LAG-3, BTLA and TIGIT. In one example, the conjugate for a dual checkpoint inhibition may comprise antagonistic antibodies specific to the T-cell co-inhibitory receptors CTLA-4 and PD-1 or its ligand (i.e., PD-L1 and PD-L2). Targeting of CTLA-4, PD-1 or its ligands may enhance T cell activation in the tumor microenvironment and can be applied in multiple immunogenic cancer types. In another example, the conjugate for a dual checkpoint inhibition may involve inhibition of PD-1 and LAG-3. Grogan et al discloses that PD-1 axis binding antagonist may be used in combination with an agent that decreases or inhibits T cell immunoreceptor with immunoglobulin and ITIM domain (TIGIT) activity (PCT publication No. 2015/009856; the contents of which are incorporated herein by reference in its entirety).

In some aspects. Compositions for enhance T cell activation may further comprises at least one agonist agent specific to a costimulatory signal molecule. The costimulatory molecule for T cell regulation may include, but are not limited to B7/CD28 family members CD28, ICOS and ICOSL (B7-H2); and tumor necrosis factor (TNF)/tumor necrosis factor receptor (TNFR) family members 4-1BB(CD137), 4-1BB (CD137L), CD27, CD70, CD40, CD40L, OX40, OX40L, CD30, CD30L, LIGHT, GITR and GITRL.

In one example, the combination modulation for immunotherapy may combine the CTLA-4 and/or PD-1 blocking with T-cell co-stimulatory receptors, particularly TNF/TNFR family members, such as CD27, CD70, CD137 and OX40. In this context, an antagonistic antibody specific to CTLA-4 and an agonistic antibody specific to CD137 may be included in the conjugate of the present invention, and may be formulated in the present nanoparticles. Such agents may include those disclosed in U.S. Pat. No. 8,475,790; the contents of which are incorporated herein by reference in its entirety.

In other example, a conjugate comprising an agonistic antibody specific to CD27 may be used in combination with antagonist agents specific to co-inhibitory molecules such as PD-1, CTLA-4, as disclosed in PCT publication NO. 2015/0167718; the contents of which are incorporated herein by reference in its entirety.

In some embodiments, compositions of the present invention may be used to inhibit the coinhibitory signals that regulate natural killer (NK) cell activation. Natural killer (NK) cells are potent immune effector cells that can respond to infection and cancer by secreting cytokines and being directly cytolytic to tumor cells (i.e. innate immune response), as well as activating antigen presentation and T cell activation (i.e. adaptive immune response). In some aspects, the conjugates used to modulate NK cell activation may comprise at least one, preferably two antagonist agents specific to MR (killer-cell immunoglobulin-like receptor), Ly49 inhibitory isoform and LIR (leukocyte inhibitory receptor).

In some aspects. Compositions for enhance NK cell activation may further comprises at least one agonist agent specific to a costimulatory signal molecule. The costimulatory molecule for NK cell regulation may include, but are not limited NKG2D and CD94—NKG2 heterodimer. The costimulatory and coinhibitory targets for NK cell activation may also include signal molecules involved in T cell regulation and also expressed on NK cells.

In some embodiments, conjugates, nanoparticles and formulations of the present invention may be used for modulating the tumor microenvironment by inhibiting or depleting the proliferation, recruitment and negative regulation on antitumor immunity of regulatory immune cells in the tumor microenvironment. The regulatory immune cells are CD+25 regulatory T cells, myeloid derived suppressor cells (MDSCs), regulatory dendritic cells, and tumor infiltrating macrophages (TAMs).

In one example, the conjugate may comprise anti-CD25 antibodies as active agents for depleting CD25+ regulatory T cells to enhance the efficacy of a variety of immunotherapy, such as various types of cancer vaccines.

In some embodiments, conjugates, nanoparticles and formulations of the present invention may be used for modulating the tumor microenvironment via regulating the activity of immunosuppressive enzymes including arginase and indoleamine-2,3-dioxygenase (IDO), or via neutralizing the inhibitory effect of tumor associated cytokines, chemokines, growth factors and other soluble factors including IL-10, TGF-β and VEGF. In some aspects, the conjugate may comprise a neutralizing antibody, and/or a functional fragment/variant thereof, of IL-10, TGF-β and VEGF.

In some embodiments the conjugate of the present invention may comprising two or more different active agents that are linked to the targeting moiety through the linker and serve as a bispecific or multiple specific conjugate.

In some embodiments, the immunomodulation therapy may be used in conjunction with other cancer immunotherapies, radiation therapies, chemotherapies, and surgery and gene therapies. The immunotherapy may be cancer vaccines including tumor associated peptide vaccines and dendritic cell vaccines, and adoptive T cell transfer therapy.

### Application

### A. Cancer Treatment

Conjugates and other compositions of the present invention may be applied for the treatment of a variety of cancers, including, but not limited to, the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma,and schwannomas; tumors of mesenchymal origin including fibrosarcoma, rhabdomyosarcoma, and osteosarcoma; other tumors including melanoma, xenoderma pigmentosum, keratoactanthoma, seminoma, thyroid follicular cancer, and teratocarcinoma; melanoma, unresectable stage III or IV malignant melanoma, squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, bone cancer, bone tumors, adult malignant fibrous histiocytoma of bone; childhood, malignant fibrous histiocytoma of bone, sarcoma, pediatric sarcoma, sinonasal natural killer, neoplasms, plasma cell neoplasm; myelodysplastic syndromes; neuroblastoma; testicular germ cell tumor, intraocular melanoma, myelodysplastic syndromes; myelodysplastic/myeloproliferative diseases, synovial sarcoma, chronic myeloid leukemia, acute lymphoblastic leukemia, philadelphia chromosome positive acute lymphoblastic leukemia (Ph+ ALL), multiple myeloma, acute myelogenous leukemia, chronic lymphocytic leukemia, mastocytosis and any symptom associated with mastocytosis, and any metastasis thereof. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, mast cell leukemia, in addition to other cancers. Other cancers are also included within the scope of disorders including, but are not limited to, the following: carcinoma, including that of the bladder, urothelial carcinoma, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma;and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma, and any metastasis thereof.

### B. Infection Diseases

Conjugates and other compositions of the present invention may be applied for the treatment of a variety of infection diseases such as bacterial, fungal, parasitic or virual infections, alone or incombination with other anti-infection medications. Examples of bacteria, viruses, fungi, and parasites which cause infection are well known in the art. An infection can be acute, subacute, chronic, or latent, and it can be localized or systemic. Compositions of the present invention may be used to increase the general immune response in a subject infected.

### Definitions

The terms used in this invention are, in general, expected to adhere to standard definitions generally accepted by those having ordinary skill in the relevant art.

About: As used herein, the term “about” means a range of normal tolerance in the art, for example within 2 standard deviations of the mean. About can be understood as within 50%, 45%, 40%, 35%, 30%, 25%, 20%, 15%, 10%, 9%, 8%, 7%, 6%, 5%, 4%, 3%, 2%, 1%, 0.5%, 0.1%, 0.05%, or 0.01% of the stated value.

Administration: As used herein, the term “administration” means the actual physical introduction of the composition into or onto (as appropriate) the host. Any and all methods of introducing the composition into the host are contemplated according to the invention; the method is not dependent on any particular means of introduction and is not to be so construed. Means of introduction are well-known to those skilled in the art, and also are exemplified herein

Adoptive cellular immunotherapy: As used herein, the terms “adoptive cellular immunotherapy” or “adoptive immunotherapy’ or “T cell immunotherapy”, or “Adoptive T cell therapy (ACT)”, are used interchangeably. Adoptive immunotherapy uses T cells that a natural or genetically engineered reactivity to a patient's cancer are generated in vitro and then transferred back into the cancer patient. The injection of a large number of activated tumor specific T cells can induce complete and durable regression of cancers.

Agonist: As used herein, the term “agonist” refers to any substance that binds to a target (e.g. a receptor); and activates or increases the biological activity of the target. For example, an “agonist” antibody is an antibody that activates or increases the biological activity of the antigen(s) it binds.

Antagonist: As used herein, the term “antagonist” refers to any agent that inhibits or reduces the biological activity of the target(s) it binds. For example, an “antagonist” antibody is an antibody that inhibits or reduces biological activity of the antigen(s) it binds.

Antigen: As used herein, the terms “antigen “or “immunogen”, as being used interchangeably, is defined as a molecule that provokes an immune response when it is introduced into a subject or produced by a subject such as tumor antigens which arise by the cancer development itself. This immune response may involve either antibody production, or the activation of specific immunologically-competent cells such as cytotoxic T lymphocytes and T helper cells, or both. An antigen can be derived from organisms, subunits of proteins/antigens, killed or inactivated whole cells or lysates. The term “antigenic” or “immunogenic” refers to a structure that is an antigen. These terms are used interchangeably.

Antigen presenting cells (APCs): As used herein, the term “antigen presenting cells” refers to cells that process antigens and present peptide epitopes on the cell surface via MHC molecules; APCs include dendritic cells (DCs), Langerhans cells, macrophages, B cells, and activated T cells. Dendritic cells (DCs) and macrophages are antigen presenting cells in vivo. The dendritic cells are more efficient APCs than macrophages. These cells are usually found in structural compartments of the lymphoid organs such as the thymus, lymph nodes and spleen, and in the bloodstream and other tissues of the body as well.

Antibodies: As used herein, “antibodies” are specialized proteins called immunoglobulins (Igs) that specifically recognize and bind to specific antigens that caused their stimulation. Antibody production by B lymphocytes in vivo and binding to foreign antigens is often critical as a means of signaling other cells to engulf, kill or remove that substance that contains the foreign antigens from the body. An immunoglobulin is a protein comprising one or more polypeptides substantially encoded by the immunoglobulin kappa and lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Also subclasses of the heavy chain are known. For example, IgG heavy chains in humans can be any of IgG1, IgG2, and IgG3 and IgG4 subclass.

Antibodies may exist as full length intact antibodies or as a number of well-characterized fragments produced by digestion with various peptidases or chemicals, such as F(ab′)2, a dimer of Fab which itself is a light chain joined to VH-CH1 by a disulfide bond; an Fab′ monomer, a Fab fragment with the hinge region; and a Fc fragment, a portion of the constant region of an immunoglobulin. An “antibody fragment” is a portion of an intact antibody such as F(ab′)a, F(ab)2, Fab′, Fab, Fv, sFv and the like. Regardless of structure, an antibody fragment binds with the same antigen that is recognized by the full-length antibody.

While various antibody fragments are defined in terms of the digestion of an intact antibody, one of skill will appreciate that any of a variety of antibody fragments may be synthesized de novo either chemically or by utilizing recombinant DNA methodology. Thus, the term antibody, as used herein also includes antibody fragments either produced by the modification of whole antibodies or synthesized de novo or antibodies and fragments obtained by using recombinant DNA methodologies. Recombinant antibodies may be conventional full length antibodies, antibody fragments known from proteolytic digestion, unique antibody fragments such as Fv or single chain Fv (scFv), domain deleted antibodies, and the like. An Fv antibody is about 50 Kd in size and comprises the variable regions of the light and heavy chain. A single chain Fv (“scFv”) polypeptide is a covalently linked VH::V_{L} heterodimer.

An antibody may be a non-human antibody, a human antibody, a humanized antibody or a chimeric antibody. The “chimeric antibody” means a genetically engineered fusion of parts of a non-human (e.g., mouse) antibody with parts of a human antibody. Generally, chimeric antibodies contain approximately 33% non-human protein and 67% human protein. Developed to reduce the HAMA response elicited by non-human antibodies, they combine the specificity of the non-human antibody with the efficient human immune system interaction of a human antibody. A human antibody may be a “fully human” antibody. The terms “human” and ‘fully human” is used to label those antibodies derived from transgenic mice carrying human antibody genes or from human cells. To the human immune system, however, the difference between “fully human” “humanized”, and “chimeric” antibodies may be negligible or nonexistent and as such all three may be of equal efficacy and safety. The term “neutralizing antibody” as used in the context of the present invention, refers to an antibody that binds to an antigen and neutralizes any effect the antigen has biologically.

Autologous: As used herein, the term “autologous” is meant to refer to any material derived from the same individual to which it is later to be re-introduced into the individual.

Cancer: As used herein, the term “cancer” refers a broad group of various diseases characterized by the uncontrolled growth of abnormal cells in the body. Unregulated cell division and growth divide and grow results in the formation of malignant tumors that invade neighboring tissues and may also metastasize to distant parts of the body through the lymphatic system or bloodstream.

Combination therapy: As used herein, the term “combination therapy” means a therapy strategy that embraces the administration of therapeutic compositions of the present invention (e.g., conjugates comprising one or more neoantigens) and one or more additional therapeutic agents as part of a specific treatment regimen intended to provide a beneficial (additive or synergistic) effect from the co-action of these therapeutic agents. Administration of these therapeutic agents in combination may be carried out over a defined time period (usually minutes, hours, days, or weeks depending upon the combination selected). In combination therapy, combined therapeutic agent may be administered in a sequential manner, or by substantially simultaneous administration.

Compound: As used herein, the term “compound”, as used herein, is meant to include all stereoisomers, geometric isomers, tautomers, and isotopes of the structures depicted. In the present application, compound is used interchangeably with conjugate. Therefore, conjugate, as used herein, is also meant to include all stereoisomers, geometric isomers, tautomers, and isotopes of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H-, 2H- and 4H-1,2,4-triazole, 1H- and 2H- isoindole, and 1H- and 2H-pyrazole. Tautomeric forms can be in equilibrium or sterically locked into one form by appropriate substitution.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Copolymer: As used herein, the term “copolymer” generally refers to a single polymeric material that is comprised of two or more different monomers. The copolymer can be of any form, such as random, block, graft, etc. The copolymers can have any end-group, including capped or acid end groups.

Cytokine: As used herein, the term “cytokine” refers to a substance secreted by certain cells of the immune system and has a biological effect on other cells. Cytokines can be a number of different substances such as interferons, interleukins and growth factors.

Cytotoxic agent: As used herein, the term “cytotoxic agent” means a substance that inhibits or prevents the function of cells and/or causes destruction of cells, such as radioactive isotopes, chemotherapeutic agents, and toxins.

Cytotoxic T cell: As used herein, the terms “cytotoxic T cell (TC)” or “cytotoxic T lymphocyte (CTL)”, or “T-killer cells”, or “CD8+ T-cell” or “killer T cell” are used interchangeably. This type of white blood cells are T lymphocytes that can recognize abnormal cells including cancer cells, cells that are infected particularly by viruses, and cells that are damaged in other ways and induce the death of such cells.

Enhance or enhancing: As used herein, the term “enhance” or “enhancing” means to increase or prolong either in potency or duration a desired effect. By way of example, “enhancing” the effect of therapeutic agents refers to the ability to increase or prolong, either in potency or duration, the effect of therapeutic agents on during treatment of a disease, disorder or condition.

Epitope: As used herein, the term “epitope” means a small peptide structure formed by contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and about 9, or about 8-15 amino acids. A T cell epitope means a peptide which can be bound by the MHC molecules of class I or II in the form of a peptide-presenting MHC molecule or MHC complex and then, in this form, be recognized and bound by native T cells, cytotoxic T-lymphocytes or T-helper cells, respectively.

Human Leukocyte Antigen (HLA): As used herein, the terms “ Human Leokocyte Antigen (HLA)”, “ HLA proteins”, “HLA antigens”, “Major Histocompatibility Complex (MEC)”, “MHC molecules”, or “MHC proteins” all refer to proteins capable of binding peptides resulting from the proteolytic cleavage of protein antigens and representing potential T-cell epitopes, transporting them to the cell surface and presenting them there to specific cells, in particular cytotoxic T-lymphocytes or T-helper cells. The major histocompatibility complex in the genome comprises the genetic region whose gene products expressed on the cell surface are important for binding and presenting endogenous and/or foreign antigens and thus for regulating immunological processes. The major histocompatibility complex is classified into two gene groups coding for different proteins, namely molecules of MHC class I and molecules of MHC class II. The molecules of the two MHC classes are specialized for different antigen sources. The molecules of MHC class I present endogenously synthesized antigens, for example viral proteins and tumor antigens. The molecules of MHC class II present protein antigens originating from exogenous sources, for example bacterial products. The cellular biology and the expression patterns of the two MHC classes are adapted to these different roles.

MHC class I molecules (called HLA class I in human) consist of a heavy chain and a light chain and are capable of binding a short peptide with suitable binding motifs, and presenting it to cytotoxic T-lymphocytes. The peptide bound by the MHC molecules of class I originates from an endogenous protein antigen. The heavy chain of the MHC molecules of class I is preferably an HLA-A, HLA-B or HLA-C monomer, and the light chain is 3-2-microglobulin.

MHC class II molecules (called HLA class II in human) consist of an α-chain and a β-chain and are capable of binding a short peptide with suitable binding motifs, and presenting it to T-helper cells. The peptide bound by the MHC molecules of class II usually originates from an extracellular of exogenous protein antigen. The α-chain and the β-chain are in particular HLA-DR, HLA-DQ, HLA-DP, HLA-DO and HLA-DM monomers.

Immune cell: As used herein, the term “immune cell” refers to a cell that is capable of participating, directly or indirectly, in an immune response. Immune cells include, but are not limited to T-cells, B-cells, antigen presenting cells, dendritic cells, natural killer (NK) cells, natural killer T (NK) cells, lymphokine-activated killer (LAK) cells, monocytes, macrophages, neutrophils, granulocytes, mast cells, platelets, Langerhan's cells, stem cells, peripheral blood mononuclear cells, cytotoxic T-cells, tumor infiltrating lymphocytes (TIL), etc. “An antigen presenting cell” (APC) is a cell that are capable of activating T cells, and includes, but is not limited to, monocytes/macrophages, B cells and dendritic cells (DCs). “Dendritic cell” or “DC” refers to any member of a diverse population of morphologically similar cell types found in lymphoid or non-lymphoid tissues. These cells are characterized by their distinctive morphology, high levels of surface MHC-class II expression. DCs can be isolated from a number of tissue sources. DCs have a high capacity for sensitizing MHC-restricted T cells and are very effective at presenting antigens to T cells in situ. The antigens may be self-antigens that are expressed during T cell development and tolerance, and foreign antigens that are present during normal immune processes. As used herein, an “activated DC” is a DC that has been pulsed with an antigen and capable of activating an immune cell. “T-cell” as used herein, is defined as a thymus-derived cell that participates in a variety of cell-mediated immune reactions, including CD8+ T cell and CD4+ T cell. “B-cell” as used herein, is defined as a cell derived from the bone marrow and/or spleen. B cells can develop into plasma cells which produce antibodies.

Immune response: As used herein, the term “immune response” means a defensive response a body develops against “foreigner” such as bacteria, viruses and substances that appear foreign and harmful. An immune response in particular is the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues. An anti-cancer immune response refers to an immune surveillance mechanism by which a body recognizes abnormal tumor cells and initiates both the innate and adaptive of the immune system to eliminate dangerous cancer cells.

The innate immune system is a non-specific immune system that comprises the cells (e.g., Natural killer cells, mast cells, eosinophils, basophils; and the phagocytic cells including macrophages, neutrophils, and dendritic cells) and mechanisms that defend the host from infection by other organisms. An innate immune response can initiate the productions of cytokines, and active complement cascade and adaptive immune response. The adaptive immune system is specific immune system that is required and involved in highly specialized systemic cell activation and processes, such as antigen presentation by an antigen presenting cell; antigen specific T cell activation and cytotoxic effect.

Immunotherapy: As used herein, the term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication, condition or biochemical indicia associated with a disease.

Immunoregulator: As used herein, the term “immunoregulator” refers to a substance, an agent, a signaling pathway or a component thereof that regulates an immune response. “Regulating,” “modifying” or “modulating” an immune response refers to any alteration in a cell of the immune system or in the activity of such cell. Such regulation includes stimulation or suppression of the immune system which may be manifested by an increase or decrease in the number of various cell types, an increase or decrease in the activity of these cells, or any other changes which can occur within the immune system. Both inhibitory and stimulatory immunoregulators have been identified, some of which may have enhanced function in the cancer microenvironment.

Linker: As used herein, the term “linker” refers to a carbon chain that can contain heteroatoms (e.g., nitrogen, oxygen, sulfur, etc.) and which may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 atoms long. Linkers may be substituted with various substituents including, but not limited to, hydrogen atoms, alkyl, alkenyl, alkynl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. Those of skill in the art will recognize that each of these groups may in turn be substituted. Examples of linkers include, but are not limited to, pH-sensitive linkers, protease cleavable peptide linkers, nuclease sensitive nucleic acid linkers, lipase sensitive lipid linkers, glycosidase sensitive carbohydrate linkers, hypoxia sensitive linkers, photo-cleavable linkers, heat-labile linkers, enzyme cleavable linkers (e.g., esterase cleavable linker), ultrasound-sensitive linkers, and x-ray cleavable linkers. Linkers may include any of those taught in, for example, WO2014/10628, the contents of which are incorporated herein by reference in their entirety.

Mean particle size: As used herein, the term “mean particle size” generally refers to the statistical mean particle size (diameter) of the particles in the composition. The diameter of an essentially spherical particle may be referred to as the physical or hydrodynamic diameter. The diameter of a non-spherical particle may refer to the hydrodynamic diameter. As used herein, the diameter of a non-spherical particle may refer to the largest linear distance between two points on the surface of the particle. Mean particle size can be measured using methods known in the art such as dynamic light scattering. Two populations can be said to have a “substantially equivalent mean particle size” when the statistical mean particle size of the first population of particles is within 20% of the statistical mean particle size of the second population of particles; for example, within 15%, or within 10%.

Modulating or modulation or to modulate: As used herein, “modulating” or “modulation” or “to modulate” generally means either reducing, decreasing, suppressing, blocking, inhibiting or antagonizing the activity of, or alternatively increasing, enhancing, or agonizing the activity of a target. In particular, “modulating” or “to modulate” can mean either reducing or inhibiting the activity of, or alternatively increasing a (relevant or intended) biological activity (e.g., anti-cancer immunity) of a target, by at least 5%, at least 10%, at least 25%, at least 50%, at least 60%, at least 70%, at least 80%, or 90% or more, compared to activity of the target in the same assay under the same conditions but without the presence of the conjugate, nanoparticle of the present invention, i.e. baseline.

The terms “monodisperse” and “homogeneous size distribution”, as used interchangeably herein, describe a population of particles, microparticles, or nanoparticles all having the same or nearly the same size. As used herein, a monodisperse distribution refers to particle distributions in which 90% of the distribution lies within 5% of the mean particle size.

Peptide: As used herein, the term “peptide” refers to a molecule composed of a series of residues, typically L-amino acids, connected one to the other, typically by peptide bonds between the α-amino and carboxyl groups of adjacent amino acids. Peptide sometimes is used interchangeably with the term “polypeptide”. Polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described. In some embodiments, peptides are less than 50 amino acids in length.

Pharmaceutically acceptable: As used herein, the term “pharmaceutically acceptable” means a component that is suitable for use with humans and/or animals without undue adverse side effects (such as toxicity, irritation, and allergic response) commensurate with a reasonable benefit/risk ratio.

Receptor: As used herein, the term “receptor” means a naturally occurring molecule or complex of molecules that is generally present on the surface of cells of a target organ, tissue or cell type.

Targeting moiety: As used herein, the term “targeting moiety” refers to a moiety that binds to or localizes to a specific locale. The moiety may be, for example, a protein, nucleic acid, nucleic acid analog, carbohydrate, or small molecule. The locale may be a tissue, a particular cell type, or a subcellular compartment. In some embodiments, a targeting moiety can specifically bind to a selected component of the targeted locale.

Tumor associated antigen (TAA): As used herein, the term “tumor associated antigen (TAA)” refers to an antigenic substance produced in tumor cells. Tumor associated antigens may be encoded by a primary open reading frame of gene products that are differentially expressed by tumors, and not by normal tissues. They may also be encoded by mutated genes, intronic sequences, or translated alternative open reading frames, pseudogenes, antisense strands, or represent the products of gene translocation events. Tumor-associated antigens (TAA) can derive from any protein or glycoprotein synthesized by the tumor cell. TAA proteins can reside in any subcellular compartment of the tumor cell; i.e., they may be membrane-bound, cytoplasmic, nuclear-localized, or even secreted by the tumor cells. A TAA may allow for a preferential recognition of tumor cells by specific T cells or immunoglobulins, therefore activate an anti-tumor immune response to kill tumor cells.

Tumor infiltrating cells: As used herein, “tumor infiltrating cells” are any type of cells that typically participates in an inflammatory response in a subject and which infiltrates tumor tissue. Such cells include tumor-infiltrating lymphocytes (TILs), macrophages, monocytes, eosinophils, histiocytes and dendritic cells.

Vaccine: As used herein, the term “vaccine” refers to a composition for generating immunity for the prophylaxis and/or treatment of diseases.

### EXAMPLES

### Example 1

### Preparation of Checkpoint Receptor Binding Conjugates

A peptide construct moiety that binds to CTLA-4 or PD1 on T cells is prepared. In some embodiments, the peptide is a single chain variable fragment (scFV) of a CTLA-4 binding antibody or a PD1 binding antibody. The binding of the peptide construct moiety to CTLA-4+ or PD1+ T cells is measured by flow cytometric analysis and/or fluorescence-activated cell sorting (FACS).

A tumor cell binding moiety is attached to the CTL-A4 or PD1 binding moiety prepared above, optionally with a linker, to make the conjugate. In some embodiments, the tumor cell binding moiety is an antagonist of SSTR2. In some emdoiments, the linker comprises a maleimide group. Example 2. Binding of the conjugates to checkpoint receptors and/or tumor cells

Studies are carried out to measure the binding of the conjugates to checkpoint receports and/or to tumor cells. Conjugates with different SSTR2-binding moieties, different CTL-A4 or PD1-binding moieties, and/or different linkers are tested in vitro to improve affinity, PK and/or T cell mediated cytotoxicity against SSTR2 expressing tumor cells.

Conjugates with which in vitro T cell mediated cytotoxicity can be demonstrated are advanced for in vivo testing, including determination of pharmacokinetic properties and antitumor efficacy. Initial efficacy testing are conducted in immunocompromised mice with co-injection of human T cells (PBMCs, peripheral blood mononuclear cells) and tumor cells followed by dosing with the conjugate of the present invention, following a protocol established for bispecific single-chain antibody (BiTE) molecules by Dreier et al. (Dreier et al., J Immunol., vol. 170:4397 (2003), the contents of which are incorporated herein by reference in their entirety).

### Equivalents and Score

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

1. A conjugate for inhibiting an immunosuppressive effect in a cancer comprising the structure of the formula X—Y—Z, wherein X is a targeting moiety; Y is a linker; and Z is an active agent that is capable of inhibiting the immunosuppressive effect.

2. The conjugates of claim 1, wherein the active agent, Z, is an antagonistic agent targeted to a coinhibitory molecule.

3. The conjugate of claim 2, wherein the coinhibitory molecule is selected from the group consisting of CTLA-4, PD-1, PD-L1, PD-L2, TIM-3, LAG-3, BTLA, CD160, C200R, TIGIT, KLRG-1, KIR, 2B4/CD244, VISTA and Ara2R.

4. The conjugate of claim 3, wherein the antagonistic agent is an antagonistic antibody of the coinhibitory molecule, or a functional fragment/variant thereof.

5. The conjugate of claim 4, wherein the coinhibitory molecule is CTLA-4.

6. The conjugate of claim 5, wherein the antagonist antibody, or the functional variant is selected from MDX-010 (ipilimumab) and tremelimumab.

7. The conjugate of claim 4, wherein the coinhibitory molecule is PD-1, PD-L1 and PD-L2.

8. The conjugate of claim 7, wherein the antagonistic antibody is an antagonistic antibody specific to PD-1, which is selected from the group consisting of 17D8, 2D3, 4H1, 5C4 (also known as nivolumab or BMS-936558), 4A11, 7D3 and 5F4 disclosed in U.S. Pat. No.: 8,008,449; AMP-224, Pidilizumab (CT-011), and Pembrolizumab.

9. The conjugate of claim 7, wherein the antagonistic antibody is an antagonistic antibody specific to PD-L1, which is selected from the group consisting of 3G10, 12A4 (also referred to as BMS-936559), 10A5, 5F8, 10H10, 1B12, 7H1, 11E6, 12B7, and 13G4 disclosed in U.S. Pat. No.: 7,943,743, MPDL3280A, MEDI4736, and MSB0010718.

10. The conjugate of claim 4, wherein the antagonistic antibody is specific to TIM-3, LAG-3, or BTLA.

11.-12. (canceled)

13. The conjugate of claim 3, wherein the antagonistic agent of the conjugate targets at least two coinhibitory molecules.

14. The conjugate of claim 13, wherein the antagonistic agent is a bispecific agent.

15. The conjugate of claim 13, wherein the antagonistic agent is a multiple specific agent.

16. The conjugate of claim 3, wherein the antagonistic agent is a non-antibody antagonist.

17. The conjugate of claim 16, wherein the non-antibody agent is a soluble polypeptide, or a fusion protein of the targeted coinhibitory molecule.

18.-19. (canceled)

20. The conjugate of claim 3, wherein the antagonistic agent is a small molecule inhibitor, or an aptamer inhibitor.

21. The conjugate of claim 3, further comprising an active agent that is an agonist of a co-stimulatory molecule.

22. The conjugate of claim 21, wherein the co-stimulatory molecule is selected from CD28, CD80 (B7.1), CD86(B7.2), 4-1BB and its ligand 4-1BBL(CD137L), CD27, CD70, CD40, CD226, CD30 and its ligand CD30L, OX40 and its ligand OX40L, GITR and its ligand GITRL, LIGHT, LTβR, LTαβ, ICOS (CD278), ICOSL (B7-H2) and NKG2D.

23. The conjugate of claim 22, wherein the agonist is an agonistic antibody specific to the costimulatory molecule, or a functional fragment/variant thereof.

24. The conjugate of claim 1, wherein the active agent is an inhibitor of arginase (ARG) and indoleamine 2,3-dioxygenase (IDO).

25. The conjugate of claim 1, wherein the active agent is an agent used to deplete a regulatory immune cell in the cancer.

26. The conjugate of claim 25, wherein the regulatory immune cell is a regulatory T cell, a myeloid derived suppressor cell, a regulatory dendritic cell, or a tumor infiltrating macrophage.

27. The conjugate of claim 26, wherein the immune cell is a regulatory T cell and wherein the active agent is an anti-CD25 antibody.

28. The conjugate of claim 1, wherein the active agent is an inhibitor of IL-10, VEGF and TGF-β.

29. The conjugate of claim 28, wherein the inhibitor is an antagonistic antibody specific to IL-10, VEGF and TGF-β.

30. The conjugate of claim 1, wherein the targeting moiety specifically binds to a tumor cell, a regulatory T cell, a myeloid derived suppressor cell, a regulatory dendritic cell, or a tumor infiltrating macrophage, a NK cell, a T cell, and a B cell.

31. The conjugate of claim 30, wherein the targeting moiety is an aptamer.

32. The conjugate of claim 30, wherein the targeting moiety is a peptide.

33. The conjugate of the claim 32, wherein the peptide is a tumor associated antigenic peptide.

34. The conjugate of claim 30, wherein the targeting moiety is an antibody or a functional fragment/variant thereof.

35. (canceled)

36. The conjugate of claim 34, wherein the antibody binds to a molecule specifically expressed in a tumor cell, tumor infiltrating macrophage, a myeloid derived suppressor cell, or a regulatory T cell.

37.-39. (canceled)

40. The conjugate of claim 1, wherein the active agent binds to a checkpoint receptor on T cells or natural killer cells.

41. The conjugate of claim 40, wherein the checkpoint receptor is selected from the group consisting of CTLA-4, PD-1, CD28, ICOS, BTLA, KIR, LAG3, CD137, OX40, CD27, CD40L, TIM3, and A2aR.

42. The conjugate of claim 41, wherein the active agent is an antibody, antagonist, or a functional fragment thereof that binds to the checkpoint receptor.

43. The conjugate of claim 42, wherein the active agent blocks the checkpoint pathway.

44. The conjugate of claim 40, wherein the targeting moiety binds to a cell surface protein on tumor cells.

45.-49. (canceled)

50. The conjugate of claim 1, wherein the targeting moiety X is targeting moiety complex comprising a target binding moiety (TBM) and a masking moiety (MM) attached to the TBM via a cleavable moiety (CM).

51. The conjugate of claim 50, wherein the MM is a peptide.

52. The conjugate of claim 50, wherein the CM is cleaved by an enzyme.

53. (canceled)

54. The conjugate of claim 50, wherein the CM is cleaved by a reducing agent.

55. The conjugate of claim 54, wherein the CM comprises a disulfide bond.

56.-57. (canceled)

58. The conjugate of claim 1, wherein the targeting moiety X is a targeting moiety complex comprising a target binding moiety (TBM) attached to a photocleavable moiety.

59. (canceled)

60. The conjugate of claim 58, wherein the photocleavable moiety is selected from nitorphenyl methyl alcohol, 1-nitrophenylethan-1-ol and substituted analogues.

61. The conjugate of claim 60, wherein the photocleavable moiety couples to hydroxy or amino residues present in the TBM.

62. (canceled)

63. The conjugate of claim 1, wherein the linker is a cleavable linker.

64. The conjugate of claim 63, wherein the linker is enzymatic-cleavable.

65. (canceled)

66. The conjugate of claim 63, wherein the linker is selected from the group consisting of an alkyl chain, a peptide, a beta-glucuronide, a self-stabilizing group, a hydrophilic group and a disulfide group.

67. (canceled)

68. The conjugate of claim 1, further comprising a reacting group that reacts with a functional group on a protein or an engineered protein or derivatives/analogs/mimics thereof.

69. The conjugate of claim 68, wherein the protein is a naturally occurring protein such as a serum or plasma protein, or a fragment thereof.

70. The conjugate of claim 69, wherein the protein is thyroxine-binding protein, transthyretin, al-acid glycoprotein (AAG), transferrin, fibrinogen, albumin, an immunoglobulin, α-2-macroglobulin, a lipoprotein, or a fragment thereof.

71. The conjugate of claim 1, further comprising a pharmacokinetic modulating unit.

72. The conjugate of claim 71, wherein the pharmacokinetic modulating unit is a natural or synthetic protein or fragment thereof, a natural or synthetic polymer, or a particle.

73. The conjugate of claim 72, wherein the pharmacokinetic modulating unit comprises a polysialic acid unit, a hydroxyethyl starch (HES) unit, or a polyethylene glycol (PEG) unit.

74. The conjugate of claim 72, wherein the pharmacokinetic modulating unit comprises dendrimers, inorganic nanoparticles, organic nanoparticles, or liposomes.

75. A nanoparticle for inhibiting an immunosuppressive effect comprising at least one conjugate for inhibiting an immunosuppressive effect comprising the conjugate of claim 1.

76. The nanoparticle of claim 75, wherein the nanoparticle comprises a polymeric matrix.

77.-79. (canceled)

80. The nanoparticle of claim 76, wherein the polymeric matrix comprises one or more polymers selected from the group consisting of poly(lactic acid), poly(glycolic acid), poly(lactic-co-glycolic acid), poly(ethylene oxide), poly(ethylene glycol), poly(propylene glycol), and copolymers thereof.

81. The nanoparticle of claim 76, wherein the size of the nanoparticle is between 10 nm and 5000 nm.

82.-84. (canceled)

85. A pharmaceutical formulation for eliciting a cancer specific immune response comprising the conjugate of claim 1.

86. A method for inhibiting an immunosuppressive signal to increase a cancer specific immune response in a subject comprising administering the subject a pharmaceutically effective amount of the conjugate of claim 1.

87. (canceled)
